(19) United States
(12) Patent Application Publication
(10) Pub. No.: US 2015/0329911 A1
(43) Pub. Date: Nov. 19, 2015

(54) NUCLEIC ACID BIOMARKERS FOR PROSTATE CANCER

(71) Applicant: INSTITUTE OF CANCER RESEARCH: ROYAL CANCER HOSPITAL, London (GB)

(72) Inventors: Graham John SPEIGHT, Oxfordshire (GB); Andrew James ROGERS, Oxfordshire (GB); Colin COOPER, Norfolk (GB)

(21) Appl. No.: 14/433,917

(22) PCT Filed: Oct. 10, 2013
(86) PCT No.: PCT/GB2013/052649

§ 371 Date: Apr. 7, 2015

(30) Foreign Application Priority Data
Oct. 10, 2012 (GB) 1218219.2
Jul. 3, 2013 (GB) 1311958.1

(51) Int. Cl. C12Q 1/68

(52) U.S. Cl. CPC C12Q 1/6886; C12Q 2600/178; C12Q 2600/118

(57) ABSTRACT

This invention relates to microRNA biomarkers useful in the diagnosis and prognosis of prostate cancer. The biomarkers are also useful for the monitoring and/or treatment of prostate cancer.

This application claims the benefit of UK applications GB 1218219.2 (filed 10 Oct. 2012) and GB 1311958.1 (filed 3 Jul. 2013), the complete contents of which are hereby incorporated herein by reference for all purposes.

### TECHNICAL FIELD

This invention relates to microRNA biomarkers useful in the diagnosis and/or prognosis of prostate cancer. The biomarkers are also useful for the monitoring and/or treatment of prostate cancer.

### BACKGROUND

Prostate cancer (PC) is a disease of the prostate, a gland in the male reproductive system. In a subset of men, PC is aggressive and this form has a high mortality. Currently, the severity of PC is measured on a clinically defined scale called the “Gleason scale” [1]. The Gleason scale ranges between 1-5 (with “1” being defined as differentiated normal healthy tissue and “5” being defined as undifferentiated, invasive tissue). Using this scale, pathologists interrogate the microscopic appearance of PC histopathological slices and grade the most common tumour pattern first, and then grade the next most common tumour pattern thereafter. These two grades are then combined to get a “Gleason score”. Areas of aggressive cancer in the prostate contain more undifferentiated tissue, which are associated with a higher Gleason score and have a greater chance of metastases; the indolent form tends to be slower growing, have a low Gleason score and therefore less clinically significant. It has been clinically defined that aggressive PC must include one of the following criteria: a Gleason score of ≧7 (4+3); a serum concentration of Prostate Specific Antigen (PSA; kallikrein 3) ≧20 ng/ml; regional- or distant-stage disease; and death due to metastatic PC [2,3]. Indolent PC, typically, is defined as having a Gleason score of ≦7 (3+4); localised-stage disease; and death due to non-PC related reasons. It is currently not possible to determine at an early stage whether the cancer is indolent or aggressive, with the only current course of action being ‘watchful waiting’, also known as ‘active surveillance’. Failure to diagnose and treat an aggressive form can result in the metastasis of the cancer to surrounding tissues and associated mortality; but over-treatment of patients with indolent PC is undesirable due to associated morbidity. Therefore, it is highly desirable to identify the aggressive forms of PC early.

Although removal of prostatic tissue and pathological examination is currently the only accurate test for PC, it is preferable to minimise the number of avoidable surgical procedures due to associated morbidity. Thus a biopsy is normally only recommended after receiving the results of an abnormal digital rectal examination (DRE) and evaluation of either the serum concentration of PSA, or urine detection of Prostate Cancer Antigen 3 (PCA3; DD3). PSA and PCA3 (both FDA approved) are currently the only two molecular markers approved for use in the context of PC diagnosis with tens of millions of these tests being performed annually, worldwide. Reported specificities for the PSA test vary but in general are much less than 50% [4]. A raised PSA level can indicate PC but it is also seen in other conditions of the prostate such as benign prostatic hypertrophy (BPH) and prostatitis. EU and US studies show that PSA should not be used as a population screening tool, and currently there is no biomarker approved for the prognosis of PC.

The poor performance of PSA has resulted in the search for alternative biomarkers for the early diagnosis of PC e.g. the PCA3 or DD3 antigens [58], serum markers of reference 9, the gene expression profiles of reference 10, the glycan profiles of reference 11, AMACR (alphamethylacyl CoA racemase), EPCA (early prostate carcinoma antigen), EPCA-2, gene promoter methylation, gene fusions including TMPRSS2:ERG, ERG/ETV1 gene rearrangements, PTEN gene loss, peptide fingerprints, metabolites including sarcosine, etc. A molecular test for PCA3 has eventually become the second FDA approved test for PC; although the reported specificity for this marker is higher than PSA (approximately 70-80%), it has a much poorer sensitivity (approximately 60-70%) [3, 4, 5] and the metrics obtained from a combination of the two tests (PSA and PCA3) is modest [12].

No current test can discriminate accurately between aggressive and indolent PC. Such a test would provide significant clinical benefit by enabling earlier active clinical management of aggressive cancers while reducing unnecessary intervention for indolent cancers.

There is a need for new tests providing improved sensitivity and specificity metrics to enable non-invasive diagnosis and/or prognosis of PC. The discriminatory power of these diagnostic/prognostic tests should be sufficiently high to support population-based screening approaches, something which PSA cannot achieve [13]. Ideally, they should be useful for the detection of PC at an early stage, and provide clinically useful prognostic information. It is an objective of the invention to meet these needs.

### DISCLOSURE OF THE INVENTION

The invention is based on the identification of correlations between PC and the presence or absence of small non-coding miRNAs. The inventors have identified miRNAs whose expression profiles can be used to indicate that a subject has PC or to predict future disease progress. The miRNAs can also distinguish between aggressive PC and indolent PC. Detection of the presence or absence of these miRNAs, and/or of changes in their levels over time, can thus be used to indicate if a subject has PC, or has the potential to develop aggressive PC. The miRNAs can therefore be considered as biomarkers of PC. Detection of these biomarkers in a subject sample can thus be used to improve the diagnosis, prognosis and monitoring of PC. Advantageously, the invention can be used to distinguish between PC and other diseases of the prostate such as BPH and prostatitis where inflammation and raised PSA levels are common. The invention can also be used as a population screening tool, and can also be used alongside known tests for PC, such as PSA and/or PCA3 tests, pathological examination (e.g. Gleason score determination), etc.

The invention provides a method for analysing a subject sample, comprising a step of determining the level of a Table 17 biomarker in the sample, wherein the level of the biomarker provides a diagnostic indicator of whether the subject has prostate cancer and/or a prognostic indicator of whether the subject has prostate cancer in the aggressive form or indolent form.

The inventors have found that the miRNAs in Table 17 are particularly useful in detecting PC. A subset of the miRNAs in Table 17 is shown in Table 1, and the inventors found that these miRNAs are present at significantly different levels in subjects with PC and without PC. Thus, a miRNA in Table 1 is particularly useful in the method for providing a diagnostic indicator. Another subset of miRNAs in Table 17 is shown in Table 2, and the inventors found that these miRNAs are present at significantly different levels in subjects with aggressive PC and indolent PC. Thus, a miRNA in Table 2 is particularly useful in the method as a prognostic indicator. As the miRNAs in Table 2 are also present at significantly different levels in subjects with PC and without PC, a miRNA in Table 2 is also useful in the method for providing a diagnostic indicator. Some markers are common to Tables 1 and 2 and these are particularly useful in a joint diagnostic/prognostic method.

Analysis of a single Table 17 biomarker can be performed, and detection of the miRNA can provide a useful diagnostic/prognostic indicator for PC even without considering any of the other Table 17 biomarkers. The sensitivity and specificity of diagnosis can be improved by combining data for multiple biomarkers. It is preferred to analyse more than one Table 17 biomarker. Analysis of two or more different biomarkers (a “panel”) can enhance the sensitivity and/or specificity of diagnosis/prognosis compared to analysis of a single biomarker. Panels can include marker(s) from Table 1 alone (e.g. a diagnostic panel), from Table 2 alone (e.g. a prognostic panel), or from both of Tables 1 and 2 (a joint diagnostic/prognostic panel).

Thus, the invention provides a method for analysing a subject sample, comprising a step of determining the levels of x different biomarkers of Table 17, wherein the levels of the biomarkers provide a diagnostic indicator of whether the subject has PC and/or a prognostic indicator of whether the subject has PC of either the indolent or aggressive form. The value of x is 2 or more e.g. 2, 3, 4, 5, 6, 7, 8, 9, 10, 11, 12, 13, 14, 15 or more (e.g. up to 35). These panels may include (i) any specific one of the 35 biomarkers in Table 17 in combination with (ii) any of the other 34 biomarkers in Table 17.

Suitable panels are described below for determining whether the subject has PC (Tables 3 to 9) and/or for determining PC prognosis (Tables 10 to 16).

Where diagnosis is the primary interest, the invention provides a method for analysing a subject sample, comprising a step of determining the levels of x different biomarkers of Table 1, wherein the levels of the biomarkers provide a diagnostic indicator of whether the subject has PC. The value of x is 2 or more e.g. 2, 3, 4, 5, 6, 7, 8, 9, 10, or more (e.g. up to 21). These panels may include (i) any specific one of the 21 biomarkers in Table 1 in combination with (ii) any of the other 20 biomarkers in Table 1.

Where prognosis is the primary interest, the invention provides a method for analysing a subject sample, comprising a step of determining the levels of x different biomarkers of Table 2, wherein the levels of the biomarkers provide a prognostic indicator of whether the subject has PC of either the indolent or aggressive form. The value of x is 2 or more e.g. 2, 3, 4, 5, 6, 7, 8, 9, 10, or more (e.g. up to 17). These panels may include (i) any specific one of the 17 biomarkers in Table 2 in combination with (ii) any of the other 16 biomarkers in Table 2.

Preferred panels have from 1 to 7 biomarkers, as using more than 7 biomarkers adds little to sensitivity and specificity.

The Table 17 biomarkers can be used in combination with one or more of: (a) known biomarkers for PC, which may or may not be miRNAs; and/or (b) other information about the subject from whom a sample was taken e.g. age, genotype, ethnicity, weight, other clinically-relevant data or phenotypic information; and/or (c) other diagnostic tests or clinical indicators for PC, which can include, but are not limited to, Gleason score, PSA levels, tumour grading (TNM score), etc. Such combinations can enhance the sensitivity and/or specificity of diagnosis and/or prognosis. Thus the invention provides a method for analysing a subject sample, comprising a step of determining:
(a) the level(s) of y Table 17 biomarker(s), wherein the levels of the biomarkers provide a diagnostic and/or prognostic indicator respectively of whether the subject has PC and whether the PC is of the indolent or aggressive form; and also one or both of:
(b) if a sample from the subject contains a known biomarker selected from the group consisting of PSA antigen, PCA3 antigen and/or mRNA, DD3 antigen and/or mRNA, AMACR antigen and/or mRNA, EPCA antigen and/or mRNA, EPCA-2 antigen and/or mRNA, gene promoter methylation, TMPRSS2:ERG gene fusions, and sarcosine (and optionally, any other known biomarkers e.g. see above); wherein detection of the known biomarker provides a second diagnostic and/or prognostic indicator of whether the subject has PC;
(c) the subject's age,
and combining the different diagnostic and/or prognostic indicators to provide an aggregate diagnostic and/or prognostic indicator of whether the subject has PC and/or whether a PC is of the indolent or aggressive form.

The samples used in (a) and (b) may be the same or different.

The value of y is 1 or more e.g. 1, 2, 3, 4, 5, 6, 7, 8, 9, 10, 11, 12, 13, 14, 15 (e.g. up to 35). When y>1 the invention uses a panel of different Table 17 biomarkers.

The invention also provides, in a method for diagnosing if a subject has PC, an improvement consisting of determining in a sample from the subject the level(s) of y biomarker(s) of Table 1, wherein the level(s) of the biomarker(s) provide a diagnostic indicator of whether the subject has PC.

The invention also provides, in a method for predicting whether a subject has indolent or aggressive PC, an improvement consisting of determining in a sample from the subject the level(s) of y biomarker(s) of Table 2, wherein the level(s) of the biomarker(s) provide a prognostic indicator of whether the subject has PC of either the indolent or aggressive form.

The invention also provides a method for diagnosing a subject as having PC, comprising steps of: (i) determining the levels of y biomarkers of Table 17 in a sample from the subject; and (ii) comparing the determination from step (i) to data obtained from samples from subjects without PC and/or from subjects with PC, wherein the comparison provides a diagnostic indicator of whether the subject has PC. The comparison in step (ii) can use a classifier algorithm as discussed in more detail below. Preferably, the biomarkers are selected from Table 1.

The invention also provides a method for monitoring development (and hence prognosis) of PC in a subject, comprising steps of: (i) determining the levels of z₁ biomarker(s) of Table 2 in a first sample from the subject taken at a first time; and (ii) determining the levels of z₂ biomarker(s) of Table 2 in a second sample from the subject taken at a second time, wherein: (a) the second time is later than the first time; (b) one or more of the z₂ biomarker(s) were present in the first sample; and (c) a change in the level(s) of the biomarker(s) in the second sample compared with the first sample indicates the state of the PC. The expression of the biomarker(s) in the second sample may be up- or down-regulated in comparison to the first sample, for example, as indicated in Table 2. The relative level(s) of the biomarker(s) indicate whether the prostate cancer is either in remission or is progressing. The combination of several bi-directional miRNA biomarkers (i.e. including one or more biomarkers that are up-regulated in the second sample relative to the first sample and one or more biomarkers that are down-regulated in the second sample relative to the first sample) can be used for diagnosis and/or prognosis. Thus, the method monitors the biomarker(s) over time, with changing levels indicating whether the disease is getting better or worse.

The disease development can be either an improvement or a worsening, and this method may be used in various ways e.g. to monitor the natural progress of a disease, or to monitor the efficacy of a therapy being administered to the subject. Thus, a subject may receive a therapeutic agent before the first time, at the first time, or between the first time and the second time.

The value of z₁ is 1 or more e.g. 1, 2, 3, 4, 5, 6, 7, 8, 9, 10, 11, 12, 13, 14, 15 (e.g. up to 17). The value of z₂ is 1 or more e.g. 1, 2, 3, 4, 5, 6, 7, 8, 9, 10, 11, 12, 13, 14, 15 (e.g. up to 17). The values of z₁ and z₂ may be the same or different. If they are different, it is usual that z₁>z₂ as the later analysis (z₂) can focus on biomarkers which were already detected in the earlier analysis; in other embodiments, however, z₂ can be larger than z₁ e.g. if previous data have indicated that an expanded panel should be used; in other embodiments z₂=z₁ e.g. so that, for convenience, the same panel can be used for both analyses. When z₁>1 or z₂>1, the biomarkers are different biomarkers.

The invention also provides a method for monitoring development of PC in a subject, comprising steps of: (i) determining the level of at least w₁ Table 2 biomarkers in a first sample taken at a first time from the subject; and (ii) determining the level of at least w₂ Table 2 biomarkers in a second sample taken at a second time from the subject, wherein: (a) the second time is later than the first time; (b) at least one biomarker is common to both the w₁ and w₂ biomarkers; (c) the level of at least one biomarker common to both the w₁ and w₂ biomarkers is different in the first and second samples, thereby indicating that the PC is progressing or regressing. Thus the method monitors the range of biomarkers over time, with a broadening in the number of detected biomarkers indicating that the disease is getting worse. As mentioned above, this method may be used to monitor disease development in various ways.

The value of w₁ is 1 or more e.g. 1, 2, 3, 4, 5, 6, 7, 8, 9, 10, 11, 12, 13, 14, 15 (e.g. up to 17). The value of w₂ is 2 or more e.g. 1, 2, 3, 4, 5, 6, 7, 8, 9, 10, 11, 12, 13, 14, 15 (e.g. up to 17). The values of w₁ and w₂ may be the same or different. If they are different, it is usual that w₂>w₁, as the later analysis should focus on a biomarker panel that is at least as wide as the number already detected in the earlier analysis. There will usually be an overlap between the w₁ and w₂ biomarkers (including situations where they are the same, such that the same biomarkers are measured at two time points) but it is also possible for w₁ and w₂ to have no biomarkers in common.

Where the methods involve a first time and a second time, these times may differ by at least 1 day, 1 week, 1 month or 1 year. Samples may be taken regularly. The methods may involve measuring biomarkers in more than 2 samples taken at more than 2 time points i.e. there may be a 3rd sample, a 4th sample, a 5th sample, etc.

The invention also provides a device for the diagnosis and/or prognosis of PC, wherein the device permits determination of the level(s) of y Table 17 biomarkers. The value of y is defined above. In some embodiments, the diagnostic device permits determination of the level(s) of biomarker(s) listed in Table 1. In some embodiments, the diagnostic device permits determination of the level(s) of biomarker(s) listed in Table 2. In some embodiments, the diagnostic device permits determination of the levels of at least one biomarker listed in Table 1 and at least one biomarker listed in Table 2. The device may also permit determination of whether a sample contains one or more of the known PC biomarkers mentioned above e.g. PSA and/or PCA3, and/or other known biomarkers listed above.

The invention also provides a kit comprising (i) a diagnostic and/or prognostic device of the invention and (ii) instructions for using the device to detect y of the Table 17 biomarkers. The value of y is defined above. The kit is useful in the diagnosis and/or prognosis of PC.

The invention also provides a kit comprising reagents for measuring the levels of x different Table 17 biomarkers. The kit may also include reagents for determining whether a sample contains one or more of the known PC biomarkers mentioned above e.g. PSA and/or PCA3, and/or other known biomarkers listed above. The value of x is defined above. The kit is useful in the diagnosis and/or prognosis of PC.

The invention also provides a kit comprising components for preparing a diagnostic device of the invention. For instance, the kit may comprise individual detection reagents for x different biomarkers, such that a selection of those x biomarkers can be prepared.

The invention also provides a product comprising (i) one or more detection reagents which permit measurement of x different Table 17 biomarkers, and (ii) a sample from a subject.

The invention also provides a software product comprising (i) code that accesses data attributed to a sample, the data comprising measurement of y Table 17 biomarkers, and (ii) code that executes an algorithm for assessing the data to represent a level of y of the biomarkers in the sample. The software product may also comprise (iii) code that executes an algorithm for assessing the result of step (ii) to provide a diagnostic and/or prognostic indicator of PC. As discussed below, suitable algorithms for use in part (iii) include support vector machine algorithms, artificial neural networks, tree-based methods, genetic programming, etc. The algorithm can preferably classify the data of part (ii) to distinguish between PC subjects and non-PC subjects based on measured biomarker levels in samples taken from such subjects. The algorithm can also preferably classify the data of part (ii) to distinguish between PC subjects with the indolent and aggressive forms of the disease based on measured biomarker levels in samples taken from such subjects. The invention also provides methods for training such algorithms.

The invention also provides a computer which is loaded with and/or is running a software product of the invention.

The invention also extends to methods for communicating the results of a method of the invention. This method may involve communicating assay results and/or diagnostic and/or prognostic results. Such communication may be to, for example, technicians, physicians or patients. In some embodiments, detection methods of the invention will be performed in one country and the results will be communicated to a recipient in a different country.

The invention also provides the use of a Table 1 biomarker as a diagnostic biomarker for PC.

The invention also provides the use of a Table 2 biomarker as a prognostic biomarker for PC.

The invention also provides the use of x different Table 17 biomarkers as biomarkers for PC diagnosis and/or prognosis. The value of x is defined above. These may include panels as defined above.

The invention also provides the use as combined biomarkers for PC of (a) at least y Table 17 biomarker(s) and (b) PSA, PCA3, DD3, AMACR, EPCA, EPCA-2, gene promoter methylation, TMPRSS2:ERG gene fusions, EGR/ETV1 gene rearrangements, PTEN gene loss, and/or sarcosine (and optionally, any other known biomarkers e.g. see above). The value of y is defined above. When y>1 the invention uses a panel of biomarkers of the invention.

### Biomarkers of the Invention

In total, thirty-eight (38) individual human miRNAs have been identified and these can be used as either diagnostic and/or prognostic PC biomarkers. Within the 38 miRNAs, 24 miRNAs are particularly useful for distinguishing between samples from subjects with PC and from subject without PC. Details of these 2124 miRNAs are given in Table 1. Other preferred miRNA marker subsets are listed in Tables 24 and 27.

Additionally, within the 35 miRNAs, 17 miRNAs are particularly useful for distinguishing between samples from subjects with aggressive PC and from subjects with indolent PC. Details of these 17 miRNAs are given in Table 2. Table 18 provides further details of the miRNA biomarkers, as provided by miRBase database (version 16, released, August 2010), such as the precursor hairpin pre-miRNA sequences and the genomic location of the miRNA gene. In some instances, multiple precursor pre-miRNAs (i.e. from different genomic locations) lead to the same mature miRNA sequence. Additionally, a single pre-miRNA precursor may lead to one or more mature miRNA sequences, such as sequences excised from the 5′ and 3′ arms of the hairpin, as indicated in Table 18. The methods of the invention can involve detecting and determining the levels of the mature miRNA sequences that are excised from 5′ and/or 3′ arms of the pre-miRNA precursor, as indicated in Tables 1 and 2.

The specific sequences in Table 18 are not limiting on the invention. The invention includes detecting and measuring the levels of polymorphic variants of these miRNAs. A database outlining in more detail the miRNAs listed herein is available: MiRBase [14, 15, 16, 17] or, in relation to target prediction, the DIANA-microT [18, 19], microRNA.org [20], miRDB [21, 22], TargetScan [23] and PicTar [24] databases.

As mentioned above, detection of a single Table 1 biomarker can provide useful diagnostic information, similarly detection of a single Table 2 biomarker can provide useful prognostic information, but each biomarker might not individually provide information which is useful i.e. a miRNA in Table 1 may be present in some, but not all, subjects with PC, additionally a miRNA in Table 2 may be present in some, but not all, subjects with aggressive PC. An inability of a single biomarker to provide universal diagnostic and/or prognostic results for all subjects does not mean that this biomarker has no diagnostic and/or prognostic utility, however, or else PSA also would not be useful; rather, any such inability means that the test results (as in all diagnostic/prognostic tests) have to be properly understood and interpreted.

To address the possibility that a single biomarker might not provide universal diagnostic and/or prognostic results, and to increase the overall confidence that an assay is giving sensitive and specific results across a disease population, it is advantageous to analyse a plurality of the Table 17 biomarkers (i.e. a panel). For instance, a negative signal for a particular Table 17 miRNA is not necessarily indicative of the absence of PC, or indolent PC (just as a low PSA concentration is not), but confidence that a subject does not have PC, or has indolent PC, increases as the number of negative results increases. For example, in the diagnosis of PC if all 35 biomarkers are tested and are negative then the result provides a higher degree of confidence than if only 1 biomarker is tested and is negative. Similarly, in the prognosis of PC if all Table 2 biomarkers are tested and are negative then the result provides a higher degree of confidence than if only 1 biomarker is tested and is negative that the subject has indolent PC. Thus biomarker panels are most useful for enhancing the distinction seen between diseased and non-diseased samples, as well as determining aggressive PC from indolent PC. As mentioned above, preferred panels have from 1 to 7 biomarkers as the burden of measuring a higher number of markers is usually not rewarded by better sensitivity or specificity. Preferred panels are given below.

Where a biomarker or panel provides a strong distinction between PC and non-PC subjects, then a method for analysing a subject sample can function as a method for diagnosing if a subject has PC. Where a biomarker or panel provides a strong distinction between aggressive PC and indolent PC, then a method of analysing a subject sample can function as a method for prognosticating as to the aggressiveness of the PC. As with many diagnostic/prognostic tests, however, and as is already known for the PSA test, a method may not always provide a definitive diagnosis and/or prognosis and so a method for analysing a subject sample can sometimes function only as a method for aiding in the diagnosis and/or prognosis of PC, or as a method for contributing to a diagnosis and/or prognosis of PC, where the method's result may imply that the subject has PC (e.g. the disease is more likely than not) and/or may confirm other diagnostic indicators (e.g. passed on clinical symptoms). Dealing with these considerations of certainty/uncertainty is well known in the diagnostic/prognostic field.

### Diagnosis and Prognosis

The invention involves diagnosis and/or prognosis of prostate cancer.

Diagnosis refers to the detection of the presence of PC in a subject. The biomarkers in Table 1 and Table 2 are present at significantly different levels in subjects with PC compared to those without PC. Thus, these biomarkers are particularly useful as diagnostic indicators for PC.

Prognosis refers to predicting the likely outcome of the disease (i.e. PC) in a subject, including the likelihood that the PC patient will suffer disease progression, including recurrence, metastatic spread, and drug resistance, and a cancer-attributable death. The presence or level of a biomarker of the invention may correlate with the risk or progression of a disease or the susceptibility of the disease to certain treatments. Thus, the detection and measurement of biomarkers of the invention over time may provide a useful means to monitor the progress of disease, including recurrence or metastatic spread, such as indicating the stage of the PC.

The biomarkers in Table 2 are present at significantly different levels in subjects with aggressive PC compared to those with indolent PC. Thus, the biomarkers in Table 2 are particularly useful as prognostic indicators. Hence, these biomarkers provide useful information for the accurate prediction of outcomes in PC patients.

Clinical parameters that have been associated with a poor prognosis of PC include advanced tumour stage, high PSA level at presentation, and a Gleason score of over 7. However, the tumour staging and Gleason score rely on identifying morphological changes of cells in tissue samples. In particular, it is difficult to morphologically differentiate between aggressive (which typically has a Gleason score of ≧7 (4+3)) and indolent PC (which typically has a Gleason score of ≦7 (3+4)). In some instances, small focal aggressive cancer cells may go undetected at the early stage. In contrast, the biomarkers of the invention are particularly useful because the invention relies on detecting miRNA biomarkers, which are molecular changes that precede cellular changes, so prognosis can be assessed at a much earlier stage. Hence, the invention improves the prognostic accuracy of PC, thereby enabling the optimal and early treatment and management of the patient.

### The Subject

The invention is used for diagnosing disease in a subject, and prognosticating as to the aggressiveness of the disease. The subject will be male. The subject will usually be at least 20 years old (e.g. >25, >30, >35, >40, >45, >50, >55, >60, >65, >70). They will usually be at least 50 years old as the risk of PC increases in these men, and for these subjects it may be appropriate to offer a screening service for Table 17 biomarkers.

The subject may be pre-symptomatic for PC or may already be displaying clinical symptoms. For pre-symptomatic subjects the invention is useful for predicting that symptoms may develop in the future if no preventative action is taken. For subjects already displaying clinical symptoms, the invention may be used to confirm or resolve another diagnosis. For pre-symptomatic subjects and/or subjects already displaying clinical symptoms, the invention may be used to confirm the prognosis of the PC, i.e. whether the PC is indolent or aggressive. The subject may already have begun treatment for PC.

In some embodiments the subject may already be known to be predisposed to development of PC e.g. due to family or genetic links. In other embodiments, the subject may have no such predisposition, and may develop the disease as a result of environmental factors e.g. as a result of exposure to particular chemicals (such as toxins or pharmaceuticals), as a result of diet [25], as a result of infection, etc.

It is the intention that the invention can be implemented relatively easily and/or cheaply, it is the intention that the invention is not restricted to being used in patients who are already suspected of having PC. Rather, it can be used to screen the general population or a high risk population e.g. men at least 20 years old, as listed above.

The subject will typically be a human being. In some embodiments, however, the invention is useful in non-human organisms e.g. mouse, rat, rabbit, guinea pig, cat, dog, horse, pig, cow, or non-human primate (monkeys or apes, such as macaques or chimpanzees). In non-human embodiments, any method used for detection of miRNAs by the invention will typically be based on the relevant non-human ortholog of the human miRNA disclosed herein. In some embodiments animals can be used experimentally to monitor the impact of a therapeutic on a particular biomarker.

### The Sample

The invention analyses samples from subjects. Many types of sample can include miRNAs suitable for detection by the invention, but the sample will typically be (homogenised) tissue and/or a body fluid. Suitable body fluids include, but are not limited to, tissue, blood, serum, plasma, saliva, prostate tissue, prostate fluid (i.e. fluid which immediately surrounds the prostate in vivo), prostatic secretions, lymphatic fluid, a wound secretion, urine, faeces, mucus, sweat, tears and/or cerebrospinal fluid. The sample is typically tissue, serum, plasma or urine.

Typically during a prostate biopsy, prostate tissue samples are obtained from: (i) all major regions of the prostate so as to ensure complete “geographic” coverage, and/or (ii) any region of the prostate that may be suspected to be cancerous, e.g. suspicious on transrectal ultrasound or magnetic resonance imaging. A common method of prostate biopsy is transrectal ultrasound-guided prostate (TRUS) biopsy. For the miRNAs of the invention that demonstrate a field-effect, selecting sample regions that are suspected to be cancerous is not essential for detecting PC.

In some embodiments, a method of the invention involves an initial step of obtaining the sample from the subject. In other embodiments, however, the sample is obtained separately from and prior to performing a method of the invention. After a sample has been obtained then methods of the invention could be performed in vitro. In other embodiments, however, a method of the invention involves detecting the presence and/or absence of the miRNA in vivo, for example, but not limited to, use of a detection probe (e.g. a radioactive probe) as a tracer for molecular imaging. Detection of biomarkers may be performed directly on a sample taken from a subject, or the sample may be treated between being taken from a subject and being analysed. For example, a blood sample may be treated to remove cells, leaving plasma containing free-circulating miRNA for analysis, or to remove cells and various clotting factors, leaving serum containing free-circulating miRNA for analysis. Faeces samples usually require physical treatment prior to miRNA detection e.g. suspension, homogenisation and centrifugation. For some body fluids, though, such separation treatments are not usually required (e.g. urine, tears or saliva) but other treatments may be used. For example, various types of sample may be subjected to treatments such as dilution, aliquoting, sub-sampling, heating, freezing, irradiation, etc. between being taken from the body and being analysed e.g. serum is usually stored, frozen prior to analysis. Also, addition of processing reagents is typical for various sample types e.g. addition of anticoagulants to blood samples.

A tissue sample can be preserved with a fixative (e.g. formalin) before it is analysed. A preserved sample can also be embedded (e.g. formalin-fixed, paraffin-embedded (FFPE) samples). Alternatively, a fresh tissue sample can be used, and this sample is fresh frozen, without fixatives.

Expression differences of any given miRNA may vary depending on the compartment being analysed (e.g. tissue vs plasma and/or serum). Typically, expression levels of a miRNA will be higher in tissue due to more cells being present in any given sample; the cells will be rich in miRNA. However, in plasma and/or serum, the miRNAs are free-circulating (due to release from the cells) and thus their concentration is greatly diluted in the surrounding (liquid) environment. However, a lower expression level in, for example, plasma doesn't mean that the miRNA is less biologically relevant. Also, any contrary expression differences may be due, in part, to miRNAs being sequestered in the cells and not released into the surrounding blood.

Preferably, the invention uses a combination of different types of sample, e.g. a prostate tissue sample and a blood sample. Thus, the invention provides a method for analysing a subject's samples, comprising: (i) determining the expression level of a biomarker of the invention in a prostate tissue sample; (ii) determining the expression level of the biomarker in a bodily fluid sample; (iii) comparing the determinations from (i) and (ii), wherein the difference between (i) and (ii) indicates that the subject has PC and/or aggressive or indolent PC. The tissue sample can be a fresh tissue sample or a preserved tissue sample. The body fluid sample can be a blood sample.

A biomarker of the invention may have different absolute expression levels in different types of sample. Thus, when the expression levels of the same biomarker in different sample types are compared against a control, different relative expression profiles may be observed.

For example, a biomarker of the invention can have opposite relative expression profiles (i.e. up-regulation as opposed to down-regulation in a PC sample compared to a control) in different sample types of the same subject. For example, a biomarker (e.g. hsa-miR-449a) can be up-regulated (e.g. PC sample vs. a control) in one sample type, e.g. prostate tissue samples, but down-regulated in another sample type, e.g. bodily fluid (e.g. blood) samples, from the same subject. This divergent behaviour can enhance diagnosis or prediction of PC when both types of sample are assessed.

A biomarker of the invention can have the same relative differential expression profile (e.g. up-regulation when comparing PC vs. a control) in various sample types. For example, a biomarker of the invention (e.g. hsa-miR-183*) can be up-regulated (when comparing PC vs. a control) in different sample types, e.g. tissue and bodily fluid (e.g. blood) samples.

The inventors have found that some biomarkers of the invention show a ‘field-effect’ within the prostate gland, whereby differential relative expression profiles (e.g. PC sample compared to a control) can be observed in samples from any part of the prostate. Hence, these biomarkers are able to detect or predict PC from a more generalised, less targeted, sampling of the prostate during a routine biopsy procedure.

For example, the inventors found that hsa-miR-3621, hsa-miR-33b*, hsa-miR-1973, hsa-miR-375, hsa-miR-182, hsa-miR-183, hsa-miR-602, hsa-miR-1291, hsa-miR-103, hsa-miR-148*, hsa-miR-182*, hsa-miR-185, hsa-miR-191, hsa-miR-210 and hsa-miR-494, hsa-miR-582 have significant relative differential expression profiles when samples from a non-PC region of a diseased prostate are compared to a suitable control sample, which does not have of clinical presentation of PC, but, additionally, these markers have non-differential expression profiles when comparing samples from different prostate regions in the same PC subject.

Thus, for miRNAs that demonstrate a field-effect, a method of the invention can include determining the expression level of a biomarker of the invention in a tissue sample from any region of the prostate, wherein the expression level of the biomarker indicates that the subject has PC and/or aggressive or indolent PC. The method can further comprise determining the expression level of the biomarker in a control, and comparing the expression levels of the biomarker in the tissue sample and in the control, wherein a difference in the expression levels indicate that the subject has PC and/or aggressive or indolent PC. The sample can be from a region suspected to be cancerous in the prostate or a region in the prostate that has not been suspected to be cancerous.

### Biomarker Detection

Table 17 lists 38 human miRNA molecules, and methods of the invention can involve detecting and determining the level of these miRNA biomarker(s) in a sample. Table 18 also includes nucleotide sequences for these miRNA molecules, but polymorphisms of miRNA are known in the art and so the invention can also involve detecting and determining the level of a polymorphic miRNA variant of these listed miRNA sequences.

Techniques for detecting specific miRNAs are well known in the art, e.g. microarray analysis and NanoString's nCounter technology, polymerase chain reaction (PCR)-based methods (e.g. reverse transcription PCR, RT-PCR), in-situ hybridisation (ISH)-based methods (e.g. fluorescent ISH, FISH), northern blotting, sequencing (e.g. next-generation sequencing), fluorescence-based detection methods, etc. Any of the detection techniques mentioned above can be used with the invention. Where prognosis is the primary interest, a quantitative detection technique is preferred, e.g. real-time quantitative PCR (qPCR), TaqMan® or SYBR® Green.

Detection of a miRNA typically involves contacting (“hybridising”) a sample with a complementary detection probe (e.g. a synthetic oligonucleotide strand), wherein a specific (rather than non-specific) binding reaction between the sample and the complementary probe indicates the presence of the miRNA of interest. In some instances, the miRNA in the sample is amplified prior to detection, e.g. by reverse transcription of the miRNA to produce a complementary DNA (cDNA) strand, and the derived cDNA can be used as a template in the subsequent PCR reaction.

Thus, the invention provides nucleic acids, which can be used, for example, as hybridization probes for specific detection of miRNA in biological samples or as single-stranded primers to amplify the miRNA.

The term “nucleic acid” in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Nucleic acid according to the invention can take various forms (e.g. single-stranded, primers, probes, labelled etc.). Primers and probes are generally single-stranded.

The nucleic acid can be identical or complementary to the mature miRNA sequences listed in Table 18, i.e. any one of SEQ ID NOs: 1-49. The nucleic acid may comprise sequences found in the miRBase database.

The nucleic acid can comprise a nucleotide sequence that has ≧50%, ≧60%, ≧70%, ≧75%, ≧80%, ≧85%, ≧90%, ≧95%, ≧96%, ≧97%, ≧98%, ≧99% or more identity to any one of SEQ ID NOs: 1-49. Identity between sequences is preferably determined by the Smith-Waterman homology search algorithm as described above.

The nucleic acid can comprise a nucleotide sequence that has ≧50%, ≧60%, ≧70%, ≧75%, ≧80%, ≧85%, ≧90%, ≧95%, ≧96%, ≧97%, ≧98%, ≧99% or more complementarity to any one of SEQ ID NOs: 1-49. The term “complementarity” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The nucleic acid may be 12 or more, e.g. 12, 13, 14, 15, 16, 17 or 18, etc. (e.g. up to 50) nucleotides in length. The nucleic acid may be 15-30 nucleotides in length, 10-25 nucleotides in length, 15-25 nucleotides in length, or 20-25 nucleotides in length.

The nucleic acid may include sequences that do not hybridise to the miRNA biomarkers, and/or amplified products thereof. For example, the nucleic acid may contain additional sequences at the 5′ end or at the 3′ end. The additional sequences can be a linker, e.g. for cloning or PCR purposes.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe. Methods for preparing fluorescent labelled probes, e.g. for fluorescent in-situ hybridisation FISH analysis, are known in the art, and FISH probes can be obtained commercially, e.g. from Exiqon.

The invention may use in-situ hybridisation (ISH)-based methods, e.g. fluorescent in-situ hybridisation (FISH). Hybridization reactions can be performed under conditions of different “stringency” followed by washing. Preferably, the nucleic acid of the invention hybridise under high stringency conditions, such that the nucleic acid specifically hybridises to a miRNA in an amount that is detectably stronger than non-specific hybridisation. Relatively high stringency conditions include, for example, low salt and/or high temperature conditions, such as provided by about 0.02-0.1 M NaCl or the equivalent, at temperatures of about 50-70° C. A stringent wash removes non-specific probe binding and overloaded probes. Relatively stringent wash conditions include, for example, low salt and/or presence of detergent, e.g. 0.02% SDS in 1× Saline-Sodium Citrate (SSC) at about 50° C.

In embodiments where multiple biomarkers are to be detected, an array-based assay or PCR format is preferable, in which a sample that potentially contains the biomarkers are simultaneously contacted with multiple oligonucleotide complementary detection probes, or PCR primers/probes (“multiplexed”) in a single reaction compartment, whereby a reaction compartment is defined as, but not limited to, a microtitre well, microfluidic chamber or detection pore. In other embodiments these multiple biomarkers could either be contacted with its complementary detection probe in separate, individual reaction compartments and/or; experiments could be separated over time and using different platform technologies in either multiplexed single reaction compartments or separate, individual reaction compartments. Microarray and PCR usage for the detection of miRNAs is well known in the art e.g. see reference 26 and reference 27. Microarrays may be prepared by various techniques, such as those disclosed in references 28, 29, & 30. Methods based on nucleic acid amplification are also well known in the art.

Methods and apparatus for detecting binding reactions on DNA microarrays are now standard in the art. Preferred detection methods are fluorescence-based detection methods. To detect biomarkers which have bound to immobilised oligonucleotide strands on a glass substrate is typical e.g. in which the target miRNA is fluorescently labelled and then is hybridised to a complementary oligonucleotide strand (probe).

An array is advantageous because it allows simultaneous detection of multiple biomarkers in a sample. Such simultaneous detection is not mandatory, however, and a panel of biomarkers can also be evaluated in series. Thus, for instance, a sample could be split into sub-samples and the sub-samples could be assayed in series. In this embodiment it may not be necessary to complete analysis of the whole panel e.g. the diagnostic indicators obtained on a subset of the panel may indicate that a patient has PC without requiring analysis of any further members of the panel. Such incomplete analysis of the panel is encompassed by the invention because of the intention or potential of the method to analyse the complete panel.

As mentioned above, some embodiments of the invention can include a contribution from known tests for PC, such as PSA and/or PCA3 tests. Any known tests can be used e.g. total PSA score, PSA velocity, the PROGENSA™ assay for urinary PCA3 mRNA, etc. Typically, PSA levels less than 4 ng/ml in blood are considered as normal, 4-10 ng/ml may warrant further investigation, and >10 ng/ml is high.

### DATA Interpretation

The invention involves a step of determining the level of Table 17 biomarker(s). In some embodiments of the invention this determination for a particular marker can be a simple yes/no determination (qualitative), whereas other embodiments may require a quantitative or semi-quantitative determination, still other embodiments may involve a relative determination (e.g. a ratio relative to another marker, or a measurement relative to the same marker in a control sample), and other embodiments may involve a threshold determination (e.g. a yes/no determination whether a level is above or below a threshold). A skilled person can easily determine the relative change (e.g. up-regulation or down-regulation) for any given miRNA marker relative to any particular control of interest (e.g. a negative control or a positive control) in any given sample (e.g. a prostate sample or a blood sample).

For example, the absolute levels of a biomarker in a particular control (e.g. a non-PC subject who has BPH) may be different from that in another control (e.g. a non-PC subject who has bladder cancer). It will be appreciated the relative differential expression profiles (e.g. up- or down-regulation or fold-changes) observed for the biomarkers of the invention (e.g. as provided in Tables 1, 2, 20, and 21 and FIGS. 2-11) might be applicable only for the specific control used in that study.

Usually biomarkers will be measured to provide quantitative or semi-quantitative results (whether as relative concentration, absolute concentration, fold-change, etc.) as this gives more data for use with classifier algorithms.

Usually the raw data obtained from an assay for determining the presence, absence, or level (absolute or relative) require some sort of manipulation prior to their use. For instance, the nature of most detection techniques means that some signal will sometimes be seen even if no miRNA is actually present and so this noise may be removed before the results are interpreted. Similarly, there may be a background level of the miRNA in the general population which needs to be compensated for. Data may need scaling or standardising to facilitate inter-experiments comparisons. These and similar issues, and techniques for dealing with them, are well known in the art.

Various techniques are available to compensate for background signal in a particular experiment. For example, replicate measurements will usually be performed (e.g. using multiple features of the same detection probe on a single array) to determine intra-assay variation and average values from the replicates can be compared (e.g. the median value of binding to replicate array features). Furthermore, standard markers can be used to determine inter-assay variation and to permit calibration and/or normalisation e.g. an array can include one or more standards for indicating whether measured signals should be proportionally increased or decreased.

For example, an assay might include a step of analysing the level of one or more control marker(s) in a sample e.g. levels of a miRNA unrelated to PC. Signal may be adjusted according to distribution in a single experiment. For instance, signals in a single array experiment may be expressed as a percentage of interquartile differences e.g. as [observed signal−25th percentile]/[75th percentile−25th percentile]. This percentage may then be normalised e.g. using a standard quantile normalisation matrix, such as disclosed in reference 31, in which all percentage values on a single array are ranked and replaced by the average of percentages for miRNAs with the same rank on all arrays. Overall, this process gives data distributions with identical median and quartile values. Data transformations of this type are standard in the art for permitting valid inter-array comparisons despite variation between different experiments.

The level of a biomarker relative to a single baseline level may be defined as a fold difference. Normally it is desirable to use techniques that can indicate a change of at least 1.5-fold e.g. ≧1.75-fold, ≧2-fold, ≧2.5-fold, ≧5-fold, etc.

As well as compensating for variation which is inherent between different experiments, it can also be important to compensate for background levels of a biomarker which are present in the general population. Again, suitable techniques are well known. For example, levels of a particular miRNA in a sample will usually be measured quantitatively or semi-quantitatively to permit comparison to the background level of that biomarker. Various controls can be used to provide a suitable baseline for comparison, and choosing suitable controls is routine in the diagnostic field. Further details of suitable controls are given below.

The measured level(s) of Table 17 biomarker(s), after any compensation/normalisation/etc., can be transformed into a diagnostic and/or prognostic result respectively in various ways. This transformation may involve an algorithm which provides a diagnostic and/or prognostic result as a function of the measured level(s). Where a panel is used then each individual biomarker may make a different contribution to the overall diagnostic and/or prognostic result and so two biomarkers may be weighted differently.

The creation of algorithms for converting measured levels or raw data into scores or results is well known in the art. For example, linear or non-linear classifier algorithms can be used. These algorithms can be trained using data from any particular technique for measuring the marker(s). Suitable training data will have been obtained by measuring the biomarkers in “case” and “control” samples i.e. samples from subjects known to suffer from PC and from subjects known not to suffer from PC, also samples from subjects known to suffer from aggressive PC and from subjects known to suffer from indolent PC. Most usefully the control samples will also include samples from subjects with a related disease which is to be distinguished from the disease of interest e.g. it is useful to train the algorithm with data from subjects with indolent PC and/or BPH subjects and/or with data from subjects with cancer(s) other than PC. The classifier algorithm is modified until it can distinguish between the case and control samples e.g. by adding or removing markers from the analysis, by changes in weighting, etc. Thus a method of the invention may include a step of analysing biomarker levels in a subject's sample by using a classifier algorithm which distinguishes between PC subjects and non-PC subjects based on measured biomarker levels in samples taken from such subjects.

Various suitable classifier algorithms are available e.g. linear discriminant analysis, naïve Bayes classifiers, regression modelling, perceptrons, support vector machines (SVM) [32] and genetic programming (GP) [33], as well as a series of statistical methods including, but not limited to, Principal Component Analysis (PCA), unsupervised hierarchical clustering and linear modelling. GP is particularly useful as it generally selects relatively small numbers of biomarkers and overcomes the problem of trapping in a local maximum which is inherent in many other classification methods. SVM-based approaches have previously been used for PC diagnosis by classifying images of prostate tissue [34,35], patient data [36], or gene expression levels [10]. Moreover, these approaches can potentially distinguish PC subjects from subjects with (i) indolent PC cancer (ii) other forms of cancer and (iii) confounding diseases such as BPH and prostatitis. The biomarkers in Table 17 can be used to train such algorithms to reliably make such distinctions. The average intensities of all oligonucleotide features on each array will be normalised to reduce technical bias (e.g. laser power variation, surface variation, input miRNA concentration, etc.) by a percentile normalisation procedure. Other methods for data normalisation suitable for the data include, amongst others, quantile normalisation [41]. Such normalisation methods are known in the art of microarray analysis. The resulting data will be analysed for any potential signatures relating to differences between patient cohorts referring to levels of statistical significance (generally p<0.05), multiple testing correction and fold changes within the expression data that could be indicative of biological effect (normally it is desirable to use techniques that can indicate a change of at least 1.5 fold e.g. >1.75 fold, >2-fold, >2.5-fold, >5-fold, etc.). The classification performance (sensitivity and specificity (S+S), Receiver Operator Curve (ROC) analysis) of any putative biomarkers will be rigorously assessed using nested cross validation and permutation analyses prior to further validation. Biological support for putative biomarkers will be sought using tools and databases including GeneSpring® (version 11.5.1), BioPAX pathway for GSEA analysis and Pathway Studio® (version 9.1).

It will be appreciated that, although there may be some biomarkers in Table 17 which always give a negative absolute signal when contacted with negative control samples (and thus any positive signal is immediately indicative of PC or aggressive PC, where applicable), it is more common that a biomarker will give at least a low absolute signal (and thus that a disease-indicating positive signal requires detection of miRNA levels above that background level). Thus references herein detecting a biomarker may not be references to absolute detection but rather (as is standard in the art) to a level above the levels seen in an appropriate negative control. Such controls may be assayed in parallel to a test sample but it can be more convenient to use an absolute control level based on empirical data, or to analyse data using an algorithm which can (e.g. by previous training) use biomarker levels to distinguish samples from disease patients vs. non-disease patients.

The level of a particular biomarker in a sample from a PC-diseased subject may be above or below the level seen in a negative control sample (i.e. from a healthy subject). The expression of miRNAs can either be up-regulated or down-regulated depending on the state of the individual. In a control population of healthy individuals there may thus be significant levels of miRNAs disclosed in Table 17 and these may occur at a significant frequency in the population. The level and frequency of these biomarkers may be altered in a disease cohort, compared with the control cohort. An analysis of the level and frequency of these biomarkers in the case and control populations may identify differences which provide diagnostic information. The level of a miRNA biomarker may increase or decrease in a PC sample, compared with a healthy sample.

The inventors found that hsa-miR-205 and hsa-miR-221 have significantly reduced levels in PC subjects compared to a sample taken from a non-PC region from the same diseased prostate, from the same subject (see Table 1). Thus, the detection of a reduced expression of one or more of these biomarkers in a subject relative to a negative control (e.g. a non-PC subject) may indicate that the subject has PC. Preferably, the sample is a fresh tissue sample.

The inventors found that hsa-miR-3621, hsa-miR-33b* and hsa-miR-1973 have significantly reduced levels in PC subjects compared to subjects who do not have PC, but have bladder cancer. Thus, the detection of a reduced expression of one or more of these biomarkers in a subject relative to a suitable control may indicate that the subject has PC. Preferably, the control is a sample from a subject who does not have PC, but may have a different disease e.g. bladder cancer. Preferably, the sample is a preserved prostate tissue sample (e.g. FFPE tissue sample).

The inventors found that hsa-miR-665, hsa-miR-582, hsa-miR-182, hsa-miR-378a, hsa-miR-96, hsa-miR-200b, hsa-miR-191, hsa-miR-429, hsa-miR-494, hsa-miR-99b*, hsa-miR-375, hsa-miR-141, hsa-miR-148*, hsa-miR-1291, hsa-miR-1973, hsa-miR-103, hsa-miR-3607-5p, hsa-miR-133b and hsa-miR-210 have significantly reduced levels in PC subjects compared to subjects who do not have PC, but have BPH. Thus, the detection of a reduced expression of one or more of these biomarkers in a subject relative to a suitable control may indicate that the subject has PC. Preferably, the control is a sample from a subject who does not have PC, but may have a different disease e.g. BPH. Preferably, the sample is a bodily fluid sample (e.g. a blood sample).

The inventors found that hsa-miR-665, hsa-miR-3621, hsa-miR-1973, hsa-miR-1291 and hsa-miR-183 have significantly reduced levels in PC subjects compared to subjects who do not have PC, but have BPH. Thus, the detection of a reduced expression of one or more of these biomarkers in a subject relative to a suitable control may indicate that the subject has PC. Preferably, the control is a sample from a subject who does not have PC, but may have a different disease e.g. BPH. Preferably, the sample is a bodily fluid sample (e.g. a blood sample). Preferably, the biomarker is any one of the group consisting of: hsa-miR-665, hsa-miR-3621, hsa-miR-1973 and hsa-miR-1291.

The inventors also found that hsa-miR-3621 and hsa-miR-665 have significantly reduced levels in subjects with aggressive PC compared to subjects who do not have PC, but have BPH. Thus, the detection of a reduced expression of any of these biomarkers in a subject relative to a suitable control may indicate that the subject has aggressive PC. Preferably, the control is a sample from a subject who does not have PC, but may have a different disease e.g. BPH. Preferably, the sample is a bodily fluid sample (e.g. blood sample).

The inventors also found that hsa-miR-3621, hsa-miR-665, hsa-miR-1291 and hsa-miR-1973 have significantly reduced levels in subjects with indolent PC compared to subjects who do not have PC, but have BPH. Thus, the detection of a reduced expression of any of these biomarkers in a subject relative to a suitable control may indicate that the subject has indolent PC. Preferably, the sample is a bodily fluid sample (e.g. blood sample).

The inventors also found that hsa-miR-3621, hsa-miR-183, hsa-miR-375, hsa-miR-665, hsa-miR-96, hsa-miR-663, hsa-miR-182, hsa-miR-494, hsa-miR-148a*, hsa-miR-1291, hsa-miR-602, hsa-miR-182*, hsa-miR-33b*, hsa-miR-1973, hsa-miR-153-1/hsa-miR-153-2, hsa-miR-141*, hsa-miR-1469, hsa-miR-1181 and hsa-miR-3607-5p have significantly increased levels in PC subjects compared to a sample taken from a non-PC region from the same diseased prostate, from the same subject (see Table 1). Thus, the detection of an increased expression of one or more of these biomarkers in a subject relative to a negative control (e.g. a non-cancerous sample) may indicate that the subject has PC. Preferably, the sample is a fresh tissue sample. Preferably, the biomarker is any of the group consisting of: hsa-miR-3621, hsa-miR-665, hsa-miR-1291, hsa-miR-1973, hsa-miR-33b*, hsa-miR-3607-5p, hsa-miR-1181, hsa-miR-1469 and hsa-miR-602.

The inventors also found that hsa-miR-153, hsa-miR-182, hsa-miR-183, hsa-miR-183*, hsa-miR-375 and hsa-miR-96 have significantly increased levels in PC subjects compared to subjects who do not have PC, but have bladder cancer. Thus, the detection of an increased expression of one or more of these biomarkers in a subject relative to a suitable control may indicate that the subject has PC. Preferably, the control is a sample from a subject who does not have PC, but may have a different disease e.g. bladder cancer. Preferably, the sample is a preserved prostate tissue sample (e.g. FFPE tissue sample). Preferably, the biomarker is hsa-miR-153 or hsa-miR-183*.

The inventors also found that hsa-miR-183*, hsa-miR-185, hsa-miR-133a-1, hsa-miR-1-1 have significantly increased levels in PC subjects compared to subjects who do not have PC, but have BPH. Thus, the detection of an increased expression of one or more of these biomarkers in a subject relative to a suitable control may indicate that the subject has PC. Preferably, the control is a sample from a subject who does not have PC, but may have a different disease e.g. BPH. Preferably, the sample is a bodily fluid sample (e.g. a blood sample). The level of a particular biomarker in a sample from a subject with aggressive PC may be above or below the level seen in a sample from a subject with indolent PC. The expression of miRNAs can either be up-regulated or down-regulated depending on the state of the PC. In a population of subjects with indolent PC, there may thus be significant levels of miRNAs disclosed in Table 17 and these may occur at a significant frequency in the population. The level and frequency of these biomarkers may be altered in aggressive PC cohort, compared with the indolent PC cohort. An analysis of the level and frequency of these biomarkers in the aggressive and indolent populations may identify differences which provide diagnostic and prognostic information. The level of miRNAs may increase or decrease in an aggressive PC sample, compared with an indolent PC sample.

The inventors found that hsa-miR-133a-1/hsa-miR-133a-2, hsa-miR-133b, hsa-miR-378a, hsa-miR-99b*, hsa-miR-1-1/hsa-miR-1-2, hsa-miR-139, hsa-miR-92b and hsa-miR-582 have significantly reduced levels in subjects with aggressive PC subjects compared to subjects with indolent PC (see Table 2). Thus, the detection of a reduced expression of one or more of these biomarkers in a subject relative to a control may indicate that the subject has aggressive PC, or that the PC is prone to progress, recur and/or metastasize. On the other hand, the detection of an increased expression of one or more of these biomarkers in a subject relative to a control may indicate that the PC is in remission. The control can be a healthy sample from the same subject, an earlier sample from the same subject or samples from healthy, non-PC cohort. Preferably, the sample is a fresh tissue sample. Preferably, the biomarker is any of the group consisting of: hsa-miR-99b*, hsa-miR-133b, hsa-miR-139, hsa-miR-378a and hsa-miR-133a-1.

The inventors also found that hsa-miR-133b has significantly reduced levels in subjects with aggressive PC compared to subjects who have indolent PC. Thus, the detection of a reduced expression of this biomarker in a subject relative to a suitable control may indicate that the subject has aggressive PC and/or that the PC is prone to progress, recur, and/or metastasize. On the other hand, the detection of an increased expression of this biomarker in a subject relative to a suitable control may indicate that the PC is in remission. The control can be a healthy sample from the same subject, an earlier sample from the same subject or samples from healthy, non-PC cohort. Preferably, the sample is a preserved prostate tissue sample (e.g. FFPE tissue sample).

The inventors also found that hsa-miR-3621 has significantly reduced levels in subjects with aggressive PC compared to subjects who have indolent PC. Thus, the detection of a reduced expression of this biomarker in a subject relative to a suitable control may indicate that the subject has aggressive PC and/or that the PC is prone to progress, recur, and/or metastasize. On the other hand, the detection of an increased expression of this biomarker in a subject relative to a suitable control may indicate that the PC is in remission. The control can be a healthy sample from the same subject, an earlier sample from the same subject or samples from healthy, non-PC cohort. Preferably, the sample is a bodily fluid sample (e.g. a blood sample). The inventors found that hsa-miR-96, hsa-miR-182*, hsa-miR-449a, hsa-miR-210, hsa-miR-429, hsa-miR-188, hsa-miR-200b, hsa-miR-183 and hsa-miR-183* have significantly increased levels in subjects with aggressive PC subjects compared to subjects with indolent PC (see Table 2). Thus, the detection of an increased expression of one or more of these biomarkers in a subject relative to a control may indicate that the subject has aggressive PC and/or that the PC is prone to progress, recur, and/or metastasize. On the other hand, the detection of a reduced expression of one or more of these biomarkers in a subject relative to a control may indicate that the PC is in remission. The control can be a healthy sample from the same subject, an earlier sample from the same subject or samples from healthy, non-PC cohort. Preferably the sample is a fresh tissue sample. Preferably, the biomarker is any of the group consisting of: hsa-miR-183*, hsa-miR-188-3p, hsa-miR-429, hsa-miR-200b, hsa-miR-182*, hsa-miR-96 and hsa-miR-183.

The inventors also found that hsa-miR-182 and hsa-miR-183 have significantly increased levels in subjects with aggressive PC compared to subjects with indolent PC. Thus, the detection of an increased expression of any of these biomarkers in a subject relative to a suitable control may indicate that the subject has aggressive PC and/or that the PC is prone to progress, recur, and/or metastasize. On the other hand, the detection of a reduced expression of any of these biomarkers in a subject relative to a suitable control may indicate that the PC is in remission. The control can be a healthy sample from the same subject, an earlier sample from the same subject or samples from healthy, non-PC cohort. Preferably, the sample is a preserved tissue sample (e.g. FFPE tissue sample).

The inventors also found that hsa-miR-582, hsa-miR-99b*, hsa-miR-449a and hsa-miR-210 have significantly increased levels in subjects with aggressive PC compared to subjects with indolent PC. Thus, the detection of an increased expression of any of these biomarkers in a subject relative to a suitable control may indicate that the subject has aggressive PC and/or that the PC is prone to progress, recur, and/or metastasize. On the other hand, the detection of a reduced expression of any of these biomarkers in a subject relative to a suitable control may indicate that the PC is in remission. The control can be a healthy sample from the same subject, an earlier sample from the same subject or samples from healthy, non-PC cohort. Preferably, the sample is a bodily fluid sample (e.g. blood sample).

The inventors also found that hsa-miR-1291, hsa-miR-1973 and hsa-miR-449a have significantly increased levels in subjects with aggressive PC compared to subjects with indolent PC. Thus, the detection of an increased expression of any of these biomarkers in a subject relative to a suitable control may indicate that the subject has aggressive PC and/or that the PC is prone to progress, recur, and/or metastasize. On the other hand, the detection of a reduced expression of any of these biomarkers in a subject relative to a suitable control may indicate that the PC is in remission. The control can be a healthy sample from the same subject, an earlier sample from the same subject or samples from healthy, non-PC cohort. Preferably, the sample is a bodily fluid sample (e.g. blood sample).

In general, therefore, a method of the invention will involve determining whether a sample contains a biomarker level which is associated with PC and/or aggressive PC. Thus a method of the invention can include a step of comparing biomarker levels in a subject's sample to levels in (i) a sample from a patient with known PC disease state, e.g. indolent or aggressive PC, (ii) a sample from a patient without PC, and/or (iii) an absolute value. The comparison provides a diagnostic and/or prognostic indicator of whether the subject has PC or aggressive PC. An aberrant level of one or more biomarker(s), as compared to known or standard expression levels of those biomarker(s) in a sample from a patient without PC, indicates that the subject has PC and/or aggressive PC.

A non-PC sample or a sample from a subject without PC can be any of: i) subject with no clinical presentation of prostate-related diseases; ii) BPH and iii) prostatitis. The non-PC sample or the sample from a subject without PC sample is preferably age-matched against the test subject. The non-PC sample or the sample from a subject without PC is preferably BPH.

The biomarkers of the invention have different relative differential expression profiles in a PC sample compared to a negative control. Pairs of these biomarkers (one is up-regulated and the other is down-regulated relative to the same control) may provide a useful way of diagnosing or predicting PC. For example, the inventors found that hsa-miR-183 is up-regulated in PC samples vs. control and hsa-miR-221 is down-regulated in PC samples vs. control, so this pair would be useful. This divergent behaviour can enhance diagnosis or prediction of PC when a pair of the biomarker is assessed in the same sample.

Thus, a method of the invention can include a step of comparing the expression levels of a first and a second biomarker of the invention in a subject's sample, wherein the first biomarker is positively associated with an increased risk in PC and the second biomarker is negatively associated with an increased risk in PC, wherein a difference in the expression levels between the first and second biomarkers indicates that the subject has PC and/or aggressive or indolent PC.

A method of the invention can include: (i) comparing the expression levels of a first biomarker of the invention in a subject's sample and a control, (ii) comparing the expression levels of a second biomarker of the invention in the same sample and the control, wherein the first biomarker is positively associated with an increased risk in PC and the second biomarker is negatively associated with an increased risk in PC, and (iii) comparing the determinations of (i) and (ii), wherein the comparison provides a diagnostic indicator of whether the subject has PC or a prognostic indicator of whether the subject has PC of either the indolent or aggressive form. Preferably, the difference in the relative expression levels in (i) and (ii) indicates that the subject has PC, and/or aggressive or indolent PC.

Where diagnosis of PC is the primary interest, if the sample is a prostate tissue sample (e.g. a fresh tissue sample), the first biomarker can be any of the group consisting of: hsa-miR-3621, hsa-miR-183, hsa-miR-375, hsa-miR-665, hsa-miR-96, hsa-miR-663, hsa-miR-182, hsa-miR-494, hsa-miR-148a*, hsa-miR-1291, hsa-miR-602, hsa-miR-182*, hsa-miR-33b*, hsa-miR-1973, hsa-miR-153-1/hsa-miR-153-2, hsa-miR-141*, hsa-miR-1469, hsa-miR-1181 and hsa-miR-3607-5p. Preferably, the first biomarker is any of the group consisting of: hsa-miR-3621, hsa-miR-665, hsa-miR-1291, hsa-miR-1973, hsa-miR-33b*, hsa-miR-3607-5p, hsa-miR-1181, hsa-miR-1469 and hsa-miR-602. The second biomarker can be hsa-miR-205 or hsa-miR-221.

When prognosis of PC is the primary interest, if the sample is a prostate tissue sample (e.g. a fresh tissue sample), the first biomarker can be any of the group consisting of: hsa-miR-96, hsa-miR-182, hsa-miR-449a, hsa-miR-210, hsa-miR-429, hsa-miR-188, hsa-miR-200b, hsa-miR-183 and hsa-miR-183*. Preferably, the first biomarker is any of the group consisting of: hsa-miR-183*, hsa-miR-188-3p, hsa-miR-429, hsa-miR-200b, hsa-miR-182*, hsa-miR-96 and hsa-miR-183. The second biomarker can be any of the group consisting of: hsa-miR-133a-1/hsa-miR-133a-2, hsa-miR-133b, hsa-miR-378a, hsa-miR-99b*, hsa-miR-1-1/hsa-miR-1-2, hsa-miR-139, hsa-miR-92b and hsa-miR-582. Preferably, the second biomarker is any of the group consisting of: hsa-miR-99b*, hsa-miR-133b, hsa-miR-139, hsa-miR-378a and hsa-miR-133a-1/hsa-miR-133a-2.

Where diagnosis of PC is the primary interest, if the sample is a bodily fluid (e.g. a blood sample), the first biomarker can be any of the group consisting of: hsa-miR-183*, hsa-miR-185, hsa-miR-133a-1, hsa-miR-1-1. The second biomarker can be any of the group consisting of: hsa-miR-665, hsa-miR-582, hsa-miR-182, hsa-miR-378a, hsa-miR-96, hsa-miR-200b, hsa-miR-191, hsa-miR-429, hsa-miR-494, hsa-miR-99b*, hsa-miR-375, hsa-miR-141, hsa-miR-148*, hsa-miR-1291, hsa-miR-1973, hsa-miR-103, hsa-miR-3607-5p, hsa-miR-133b and hsa-miR-210.

Where prognosis of PC is the primary interest, if the sample is a bodily fluid (e.g. a blood sample), the first biomarker can be any of the group consisting of: hsa-miR-582, hsa-miR-99b*, hsa-miR-449a and hsa-miR-210.

Where diagnosis of PC is the primary interest, if the sample is a bodily fluid (e.g. a blood sample), the second biomarker can be any of the group consisting of: hsa-miR-665, hsa-miR-3621, hsa-miR-1973, hsa-miR-1291 and hsa-miR-183. Preferably, the second biomarker is any of the group consisting of: hsa-miR-665, hsa-miR-3621, hsa-miR-1973 and hsa-miR-1291.

Where prognosis of PC is the primary interest, if the sample is a bodily fluid (e.g. a blood sample), the first biomarker can be any of the group consisting of: hsa-miR-1291, hsa-miR-1973 and hsa-miR-449a. The second biomarker can be hsa-miR-3621.

Where diagnosis of PC is the primary interest, if the sample is a prostate tissue sample (e.g. a preserved tissue sample such as FFPE tissue sample), the first biomarker can be any of the group consisting of: hsa-miR-153, hsa-miR-182, hsa-miR-183, hsa-miR-183*, hsa-miR-375, hsa-miR-96. Preferably, the biomarker is hsa-miR-153 or hsa-miR-183*. Preferably, the first biomarker is hsa-miR-153 or hsa-miR-183*. The second biomarker can be any of the group consisting of: hsa-miR-3621, hsa-miR-33b* and hsa-miR-1973. Where prognosis of PC is the primary interest, if the sample is a prostate tissue sample (e.g. a preserved tissue sample such as FFPE tissue sample), the first biomarker can be hsa-miR-183 or hsa-miR-182. The second biomarker can be hsa-miR-133b.

The level of a biomarker should be different from that seen in a control. Advanced statistical tools can be used to determine whether two levels are the same or different. For example, an in vitro diagnosis/prognosis will rarely be based on comparing a single determination. Rather, an appropriate number of determinations will be made with an appropriate level of accuracy to give a desired statistical certainty with an acceptable sensitivity and/or specificity. Levels of miRNAs can be measured quantitatively to permit proper comparison, and enough determinations will be made to ensure that any difference in levels can be assigned a statistical significance to a level of p≦0.05 or better. The number of determinations will vary according to various criteria (e.g. the degree of variation in the baseline, the degree of up-regulation in disease states, the degree of noise, etc.) but, again, this falls within the normal design capabilities of a person of ordinary skill in this field. For example, interquartile differences of normalised data can be assessed, and the threshold for a positive signal (i.e. indicating the presence or absence of a particular miRNA) can be defined as requiring that miRNAs in a sample hybridise with the complementary detection probe with at least a log change +/−0.585 than the interquartile difference above the 75th percentile. Other criteria are familiar to those skilled in the art and, depending on the assays being used, they may be more appropriate than quantile normalisation. Other methods to normalise data include data transformation strategies known in the art e.g. scaling, log normalisation, median normalisation, etc.

The underlying aim of these data interpretation techniques is to distinguish between the presence of a Table 17 biomarker and of an arbitrary control biomarker, and/or to distinguish between the response of sample from a PC and/or aggressive PC subject respectively from a control subject. Methods of the invention may have sensitivity of at least, but not limited to, 50% (e.g. >50%, >55%, >60%, 65%, >70%, >75%, >80%, >85%, >90%, >95%, >96%, >97%, >98%, >99%). Methods of the invention may have specificity of at least, but not limited to, 50% (e.g. >50%, >55%, >60%, 65%, >70%, >75%, >80%, >85%, >90%, >95%, >96%, >97%, >98%, >99%).

Data obtained from methods of the invention, and/or diagnostic and/or prognostic information based on those data, may be stored in a computer medium (e.g. in RAM, in non-volatile computer memory, on CD-ROM, DVD) and/or may be transmitted between computers e.g. over the Internet.

If a method of the invention indicates that a subject has PC, further steps may then follow. For instance, the subject may undergo confirmatory diagnostic procedures, such as those involving physical inspection of the subject, and/or may be treated with therapeutic agent(s) suitable for treating PC and/or aggressive PC.

If a method of the invention indicates that a subject has indolent PC, the subject will be treated with appropriate clinical treatments, e.g. active surveillance (i.e. put on a watch list).

If a method of the invention indicates that a subject has aggressive PC, the subject will be treated with appropriate clinical treatments, e.g. prostatectomy and/or chemotherapy.

### Monitoring the Efficacy of Therapy

As mentioned above, some methods of the invention involve testing samples from the same subject at two or more different points in time. In general, where the above text refers to the presence or absence of biomarker(s), the invention also includes an increasing or decreasing level of the biomarker(s) over time. Methods which determine changes in biomarker(s) over time can be used, for instance, to monitor the efficacy of a therapy being administered to the subject (e.g. in theranostics). The therapy may be administered before the first sample is taken, at the same time as the first sample is taken, or after the first sample is taken.

The invention can be used to monitor a subject who is receiving PC therapy. Current therapies for PC include chemotherapy and/or hormone therapy. Hormone therapy seeks to block access of dihydrotestosterone (DHT) to prostate cells or to block the effects of DHT within prostate cells. Anti-androgens are medications such as flutamide, bicalutamide, nilutamide, and cyproterone acetate which directly block the actions of testosterone and DHT within prostate cancer cells. They may be given in combination with drugs such as ketoconazole and aminoglutethimide which block the production of adrenal androgens.

In related embodiments of the invention, the results of monitoring a therapy are used for future therapy prediction. For example, if treatment with a particular therapy is effective in reducing or eliminating disease symptoms in a subject, and is also shown to decrease levels of a particular biomarker in that subject, detection of that biomarker in another subject may indicate that this other subject will respond to the same therapy. Conversely, if a particular therapy was not effective in reducing or eliminating disease symptoms in a subject who had a particular biomarker or biomarker profile, detection of that biomarker or profile in another subject may indicate that this other subject will also fail to respond to the same therapy.

In other embodiments, the presence of a particular biomarker can be used as the basis of proposing or initiating a particular therapy (patient stratification). For instance, if it is known that levels of a particular miRNA can be reduced by administering a particular therapy then that miRNA's detection may suggest that the therapy should begin. Thus the invention is useful in a theranostic setting.

Normally at least one sample will be taken from a subject before a therapy begins.

### Imaging and Staining

The miRNAs listed in Table 17 can be useful for imaging. A labelled, synthetic miRNA complementary to a miRNA(s) listed in Table 17, could be used for the identification, in ex vivo (e.g. tissue samples taken from biopsies), and in vivo (e.g. magnetic resonance imaging (MRI), positron emission tomography (PET) computed tomography (CT) scans of patients) samples of miRNAs associated with PC and/or aggressive PC. This may potentially offer a method for the early identification of PC and/or aggressive PC. Imaging techniques can also be used to monitor the progress or remission of disease, or the impact of a therapy.

The miRNAs listed in Table 17 can be useful for analysing tissue samples by staining e.g. using standard FISH. A fluorescently labelled miRNA, complementary in sequence to the miRNAs outlined in Table 17 can be contacted with a tissue sample to visualise the location of the miRNA. A single sample could be stained against multiple miRNAs, and these different miRNAs may be differentially labelled to enable them to be distinguished. As an alternative, a plurality of different samples can each be stained with a single, labelled miRNA.

Thus the invention provides a labelled nucleic acid which can hybridise to miRNA(s) listed in Table 17. The miRNA may be, but not limited to, a human miRNA, as discussed above. Any suitable label can be used e.g. quantum dots, spin labels, fluorescent labels, dyes, etc. These labelled miRNAs can be used in methods of in vivo and/or in vitro imaging.

microRNA-Based Therapy

The miRNAs listed in Table 17 can be useful for miRNA-based therapy, e.g., antisense therapy. There is literature precedent outlining the use of antisense therapy to manage cancer [37]. A synthetic miRNA complementary to a miRNA(s) listed in Table 17 could be used to stimulate cell death of cancerous cells (either associated with PC and/or aggressive PC). Additionally, in vivo antisense therapy could be used to introduce miRNA complementary to a miRNA(s) listed in Table 17 to specifically bind to, and abrogate, overexpression of specific miRNA(s) associated with PC and/or aggressive PC.

Thus the invention provides a nucleic acid which hybridises to miRNA(s) listed in Table 17 and which is conjugated to a cytotoxic agent. The miRNA may be, but not limited to, a human miRNA, as discussed above. Any suitable cytotoxic agent can be used. These conjugates miRNAs can be used in methods of therapy.

Thus the invention provides a complementary miRNA which recognises a miRNA(s) listed in Table 17 for the purposes of miRNA-based therapies which include, but not limited to, antisense therapy.

### Alternative Biomarkers

The invention has been described above by reference to miRNA biomarkers. In addition to these biomarkers, however, the invention can be used with other biological manifestations of the Table 17 miRNAs. For example, the expression level of mRNA transcripts which are a target of a Table 17 miRNA can be measured, particularly in tissues where changes in transcription level can easily be determined (such as in the potential disease tissue). Similarly, the copy number variation of a chromosomal location of a Table 17 miRNA can be measured e.g. to check for a chromosomal deletion or duplication events. The level of a regulator of transcription for a Table 17 miRNA can be measured e.g. the methylation status of the miRNA chromosomal region.

A single pre-miRNA precursor may lead to one or more mature miRNA sequences, such as sequences excised from the 5′ and 3′ arms of the hairpin, as shown in Table 18. The invention can be used to look for other mature miRNA sequences from the same pre-miRNA precursor. For example, other mature miRNA sequences from the same precursor in Table 18 may be appropriate biomarkers as well.

Further possibilities will be apparent to the skilled reader.

### Preferred Panels

Preferred embodiments of the invention are based on a panel of biomarkers. Panels of particular interest for the diagnosis of PC consist of or comprise the combinations of biomarkers listed in Tables 3 to 9 (which show seven panels of 1, 2, 3, 4, 5, 6 and 7). Panels of particular interest for the prognosis of aggressive PC consist of or comprise the combinations of biomarkers listed in Tables 10 to 16 (which show seven panels of 1, 2, 3, 4, 5, 6 and 7).

The seven different panels listed in each of Tables 3 to 9 and Tables 10 to 16 can be expanded by adding further biomarker(s) to create a larger panel. The further biomarkers can usefully be selected from known biomarkers (such as PSA, PCA3, DD3, AMACR, EPCA, EPCA-2, sarcosine, etc.; see above), from Table 17, or from Table 1, or from Table 2 where appropriate. In general the addition does not decrease the sensitivity or specificity of the panel shown in the Tables.

Such panels include, but are not limited to:
A panel comprising a biomarker selected from Table 3.
A panel comprising a biomarker selected from Table 10.
A panel comprising or consisting of 2 different biomarkers, namely: (i) a biomarker selected from Table 3 and (ii) a further biomarker selected from Table 1.
A panel comprising or consisting of 2 different biomarkers, namely: (i) a biomarker selected from Table 3 and (ii) a further biomarker selected from Table 2.
A panel comprising or consisting of 2 different biomarkers, namely: (i) a biomarker selected from Table 10 and (ii) a further biomarker selected from Table 2.
A panel comprising or consisting of 2 different biomarkers, namely: (i) a biomarker selected from Table 10 and (ii) a further biomarker selected from Table 1.
A panel comprising or consisting of 3 different biomarkers, namely: (i) a group of 2 biomarkers selected from Table 4 and (ii) a further biomarker selected from Table 1.
A panel comprising or consisting of 3 different biomarkers, namely: (i) a group of 2 biomarkers selected from Table 4 and (ii) a further biomarker selected from Table 2.
A panel comprising or consisting of 3 different biomarkers, namely: (i) a group of 2 biomarkers selected from Table 11 and (ii) a further biomarker selected from Table 2.
A panel comprising or consisting of 3 different biomarkers, namely: (i) a group of 2 biomarkers selected from Table 11 and (ii) a further biomarker selected from Table 1.
A panel comprising or consisting of 4 different biomarkers, namely: (i) a group of 3 biomarkers selected from Table 5 and (ii) a further biomarker selected from Table 1.
A panel comprising or consisting of 4 different biomarkers, namely: (i) a group of 3 biomarkers selected from Table 5 and (ii) a further biomarker selected from Table 2.
A panel comprising or consisting of 4 different biomarkers, namely: (i) a group of 3 biomarkers selected from Table 12 and (ii) a further biomarker selected from Table 2.
A panel comprising or consisting of 4 different biomarkers, namely: (i) a group of 3 biomarkers selected from Table 12 and (ii) a further biomarker selected from Table 1.
A panel comprising or consisting of 5 different biomarkers, namely: (i) a group of 4 biomarkers selected from Table 6 and (ii) a further biomarker selected from Table 1.
A panel comprising or consisting of 5 different biomarkers, namely: (i) a group of 4 biomarkers selected from Table 6 and (ii) a further biomarker selected from Table 2.
A panel comprising or consisting of 5 different biomarkers, namely: (i) a group of 4 biomarkers selected from Table 13 and (ii) a further biomarker selected from Table 2.
A panel comprising or consisting of 5 different biomarkers, namely: (i) a group of 4 biomarkers selected from Table 13 and (ii) a further biomarker selected from Table 1.
A panel comprising or consisting of 6 different biomarkers, namely: (i) a group of 5 biomarkers selected from Table 7 and (ii) a further biomarker selected from Table 1.
A panel comprising or consisting of 6 different biomarkers, namely: (i) a group of 5 biomarkers selected from Table 7 and (ii) a further biomarker selected from Table 2.
A panel comprising or consisting of 6 different biomarkers, namely: (i) a group of 5 biomarkers selected from Table 14 and (ii) a further biomarker selected from Table 2.
A panel comprising or consisting of 6 different biomarkers, namely: (i) a group of 5 biomarkers selected from Table 14 and (ii) a further biomarker selected from Table 1.
A panel comprising or consisting of 7 different biomarkers, namely: (i) a group of 6 biomarkers selected from Table 8 and (ii) a further biomarker selected from Table 1.
A panel comprising or consisting of 7 different biomarkers, namely: (i) a group of 6 biomarkers selected from Table 8 and (ii) a further biomarker selected from Table 2.
A panel comprising or consisting of 7 different biomarkers, namely: (i) a group of 6 biomarkers selected from Table 15 and (ii) a further biomarker selected from Table 2.
A panel comprising or consisting of 7 different biomarkers, namely: (i) a group of 6 biomarkers selected from Table 15 and (ii) a further biomarker selected from Table 1.
A panel comprising or consisting of a group of 7 different biomarkers selected from Table 9. This panel is particularly useful for diagnosis.
A panel comprising or consisting of a group of 7 different biomarkers selected from Table 16. This panel is particularly useful for prognosis.

Preferred panels have between 1 and 7 biomarkers in total.

### General

The term “comprising” encompasses “including” as well as “consisting” e.g. a composition “comprising” X may consist exclusively of X or may include something additional e.g. X+Y.

References to a miRNA's ability to “hybridise” to a complementary oligonucleotide probe means that the miRNA and the complementary oligonucleotide probe interact strongly enough to withstand standard washing procedures in the assay in question. Thus non-specific binding will be minimised or eliminated.

References to a “level” of a biomarker mean the amount of an analyte (e.g. a miRNA) measured in a sample and this encompasses relative and absolute concentrations of the analyte, analyte titres, relationships to a threshold, rankings, percentiles, etc.

An assay's “sensitivity” is the proportion of true positives which are correctly identified i.e. the proportion of PC subjects who test positive by a method of the invention. This can apply to individual biomarkers, panels of biomarkers, single assays or assays which combine data integrated from multiple sources e.g. PSA score and DRE. It can relate to the ability of a method to identify samples containing a specific analyte (e.g. miRNAs) or to the ability of a method to correctly identify samples from subjects with PC.

An assay's “specificity” is the proportion of true negatives which are correctly identified i.e. the proportion of subjects without PC who test negative by a method of the invention. This can apply to individual biomarkers, panels of biomarkers, single assays or assays which combine data integrated from multiple sources e.g. PSA score and DRE. It can relate to the ability of a method to identify samples containing a specific analyte (e.g. miRNAs) or to the ability of a method to correctly identify samples from subjects with PC.

Unless specifically stated, a method comprising a step of mixing two or more components does not require any specific order of mixing. Thus components can be mixed in any order. Where there are three components then two components can be combined with each other, and then the combination may be combined with the third component, etc.

References to a percentage sequence identity between two miRNA sequences means that, when aligned, that percentage of nucleotides are the same in comparing the two sequences. This alignment and the percent homology or sequence identity can be determined using software programs known in the art, for example those described in section 7.7.18 of ref. 38. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 39.

In all embodiments of the invention, where only one biomarker is used, the biomarker is preferably not hsa-miR-205, hsa-miR-183, hsa-miR-182*, hsa-miR-182, hsa-miR-449a, hsa-miR-210, hsa-miR-96 or hsa-miR-375. In all embodiments of the invention, where a panel comprises any of: hsa-miR-205, hsa-miR-183, hsa-miR-182*, hsa-miR-182, hsa-miR-449a, hsa-miR-210, hsa-miR-96 and hsa-miR-375, preferably the panel further comprises one or more biomarkers from Table 17 that is not any of hsa-miR-205, hsa-miR-183, hsa-miR-182*, hsa-miR-182, hsa-miR-449a, hsa-miR-210, hsa-miR-96 and hsa-miR-375.

### BRIEF DESCRIPTION OF DRAWINGS

FIG. 1 is a hierarchical plot showing clustering of miRNAs according to the type of tissue (i.e. disease v. normal).

FIG. 2 is a box-plot showing fold-changes of 9 miRNAs in PC samples relative to non-PC samples. Left to right (in pairs): hsa-miR183, hsa-miR183*, hsa-miR-205, hsa-miR-222, hsa-miR-224, hsa-miR-23b*, hsa-miR-31, hsa-miR-31* and hsa-miR-96. Left of the pair: normal/healthy samples, right of the pair: PC samples. Unpaired t-test: P<0.01; fold change>3.

FIG. 3 is a box plot showing fold-changes of 7 miRNAs in normal/healthy samples, indolent samples and aggressive samples. The intensity is normalised across all samples for the 7 markers. Left to right (in quintets): hsa-miR-31, hsa-miR-221, hsa-miR-222, hsa-miR-181b, hsa-miR-182, hsa-miR-183 and hsa-miR-375. Quintet from left to right: Gleason scores: 0+0, 3+3, 3+4, 4+3 and 4+5. Normal/healthy samples binned as 0+0, indolent samples binned as 3+3 and 3+4, and aggressive samples binned as 4+3 and 4+5.

FIGS. 4-11 are box-plots showing the relative expression profiles of various miRNAs between Gleason 6 (indolent PC) samples, Gleason 8 (aggressive PC) samples and PC negative samples (prostate samples derived from a subject with bladder cancer, but not prostate cancer). The miRNA analysed are: hsa-miR-3621 (FIG. 4), hsa-miR-33b* (FIG. 5), hsa-miR-182 (FIG. 6), hsa-miR-1973 (FIG. 7), hsa-miR-183* (FIG. 8), hsa-miR-153-1/hsa-miR-153-2 (FIG. 9), hsa-miR-96 (FIG. 10) and hsa-miR-133b (FIG. 11). “Gleason 6 (A)” denotes the primary cancer region in the Gleason 6 (indolent) PC sample; “Gleason 6 (E)” denotes an exclusively non-cancerous region in the Gleason 6 (indolent) PC sample; “Gleason 8 (A)” denotes a primary cancer region in the Gleason 8 (aggressive) PC sample; “Gleason 8 (E)” denotes an exclusively non-cancerous region in the Gleason 8 (aggressive) PC sample.

### MODES FOR CARRYING OUT THE INVENTION

### Array Preparation

For microarray fabrication and usage, Agilent Technologies' (“Agilent”) miRNA microarray was used. The content of the microarray is continuously aligned with releases from the miRBase database [14, 15, 16, 17], representing all known miRNAs from human beings, as well as all know human viral miRNAs. These arrays are printed using Agilent's ink-jet in situ synthesis microarray fabrication machines.

### Biomarker Confirmation

Tissue samples were obtained from radical prostatectomy, and divided into tissue slices. Within any given slice, there may be areas of cancer (“disease”) surrounded by non-cancerous tissue (“non-disease”). The aggressive and indolent samples were identified based on Gleason scores: indolent is defined as a Gleason score ≦3+4, and aggressive is defined as a Gleason score ≧4+3. Using these tissue slices two groups of samples were used:
1. disease tissue (n=83).
2. non-disease tissue (n=45).

The tissue slices were homogenised and total RNA extracted and miRNA enriched using standard column filtration methodologies, which are well known in the art. Tissue samples from both groups were individually analysed using the Agilent miRNA microarray (G4870A-031181), according to their standard protocol, (manual part number G4170-90011, version 2.4). However, deviations from the standard protocol included labelling of the samples using 2.25 μl Cyanine 3-pCp, and hybridising the microarray slides for 44 hours.

The probed and dried arrays were then scanned using a microarray scanner capable of using an excitation wavelength suitable for the detection of the labelled miRNAs, and to determine magnitude of miRNA binding to the complementary detection probe. The microarray scans produced images for each array that were used to determine the intensity of fluorescence bound to each oligonucleotide spot which were used to normalise and score array data.

The raw microarray scan image contains raw signal intensity (also referred to as the relative fluorescent unit, RFU) for each oligonucleotide spot (also referred to as a feature) on the array. These images were then feature extracted using Agilent's proprietary feature extraction software. Alternative analyses use other measures of spot intensity such as the mean fluorescence, total fluorescence, as known in the art.

The resulting average intensities of all oligonucleotide features on each array were then normalised to reduce the influence of technical bias (e.g. laser power variation, surface variation, input miRNA concentration, etc.) by a percentile normalisation procedure. Other methods for data normalisation suitable for the data include, amongst others, quantile normalisation [31]. Such normalisation methods are known in the art of microarray analysis.

Logistic regression, with optimisation for S+S was applied to all combinations of markers exceeding defined cut-offs for statistical significance and fold change. The number of biomarkers in each panel was limited to n where n=1−7. The performance of the derived panels was then ranked by combined S+S.

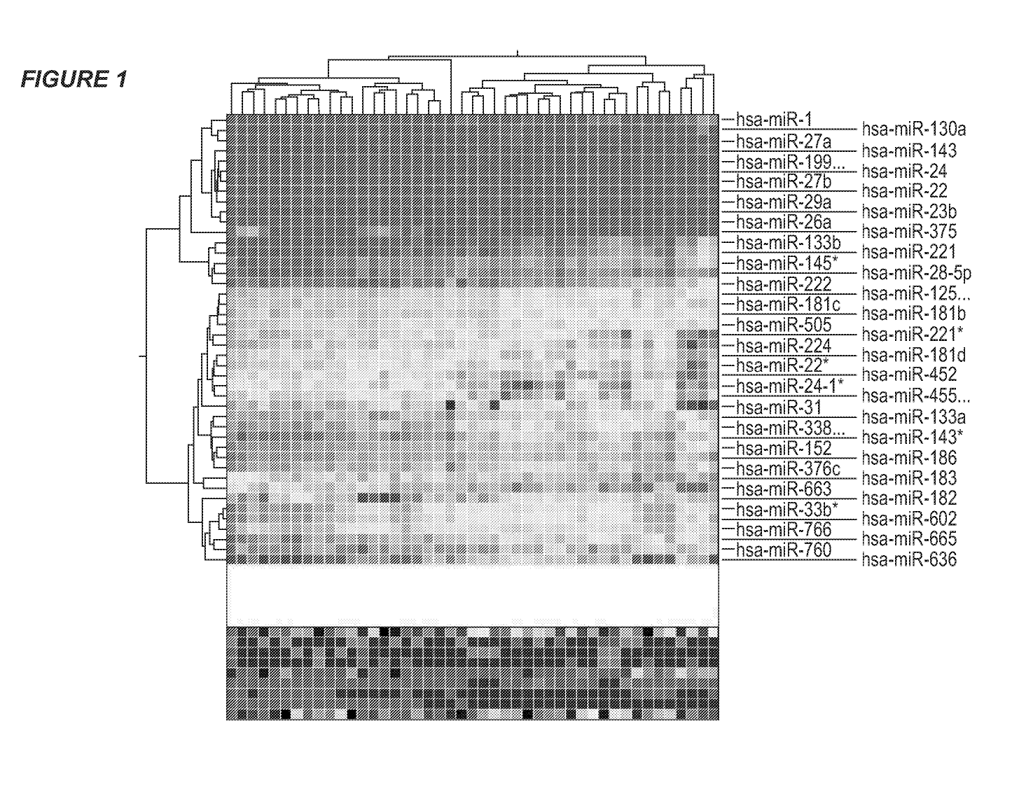

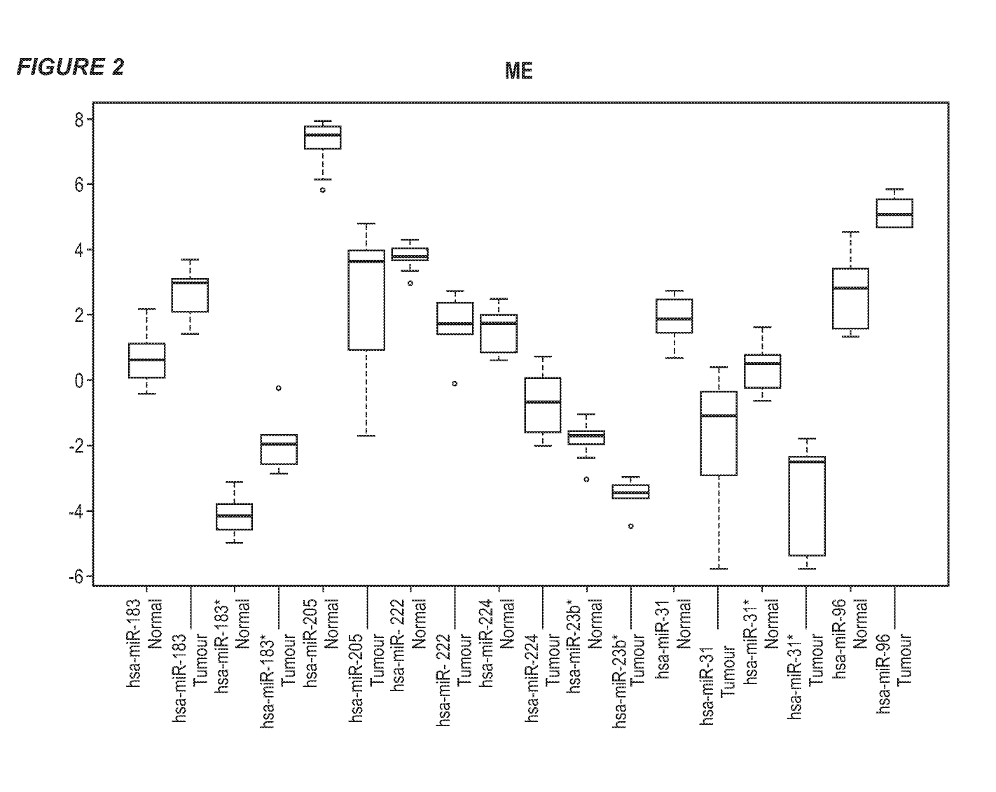

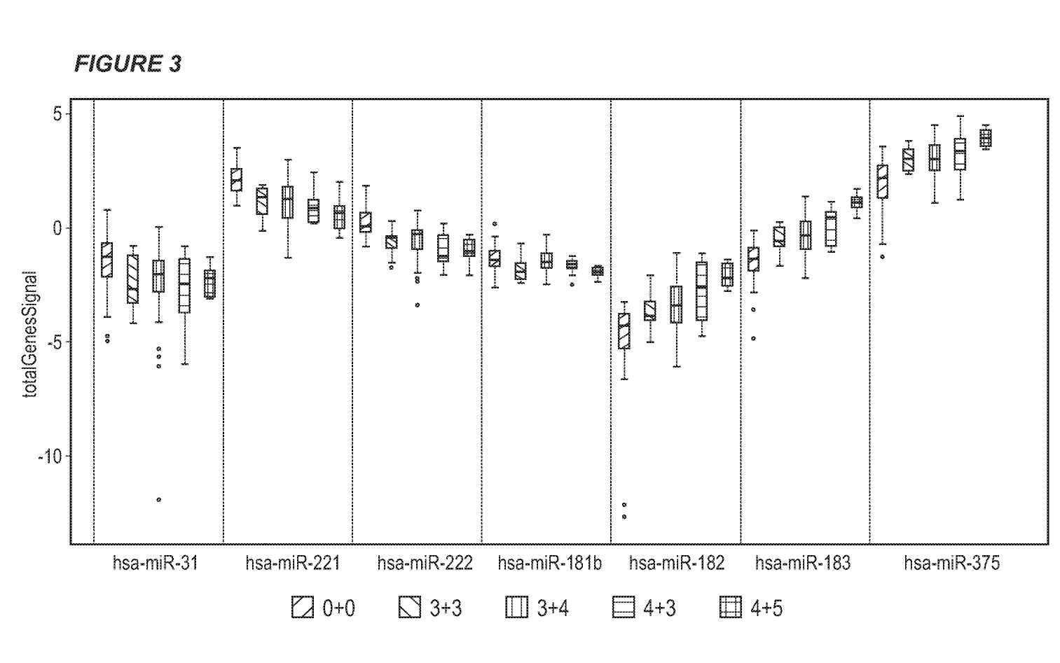

The hierarchical clustering of miRNAs according to the type of tissue (i.e. disease v. normal) is shown in FIG. 1. FIG. 2 shows expression of miRNAs in PC samples and non-PC samples. FIG. 3 shows expression of miRNAs in normal/healthy samples, indolent samples and aggressive samples.

Biomarkers from Plasma and Serum Samples

It is known that miRNA biomarkers can be found in plasma or serum of cancer patient samples (e.g. references 40,41, etc.). The inventors therefore investigated the diagnostic and/or prognostic potential of a subset of the miRNA biomarkers from Tables 1 and 2 for identifying PC in serum and plasma samples.

A set of fifty (50) prostate cancer plasma samples (9×Normal (BPH; age-matched controls); 27×Gleason 6; and 14×Gleason 8) were investigated to assess whether the miRNA markers described herein had biological utility within a different biological specimen (i.e. human plasma).

Additionally, a further set of sixty-seven (67) prostate cancer serum samples (31×Normal (age-matched controls); 31×Gleason 6; and 5×Gleason 8) were investigated to assess whether the miRNA markers described herein had biological utility within human serum.

Both the plasma and serum samples were ethically obtained from Caucasian male donors whom had been clinically assessed for their disease status. The plasma samples defined as “normal [BPH]” were classified as such based on the donor's absence of clinical symptoms associated with PC, but these donors still exhibited clinical indications associated with prostate dysfunction. For the serum samples defined as “normal”, these were classified as such based on the donor's absence of clinical symptoms associated with PC. Both sets of normal samples were closely age matched (+/−5 years) to the ages of the cancer (Gleason 6 and Gleason 8) samples. For both the plasma and serum samples, the samples defined as Gleason 8 (aggressive) and Gleason 6 (indolent) were also classified by clinical assessment (e.g. biopsy, DRE etc.) and the donors were determined to have symptoms associated with PC.

All the plasma and serum samples were processed to extract total RNA, including the small RNA fraction (<20 nt), using standard column filtration methodologies, which are well known in the art. Serum samples from all three groups were analysed using the Life Technologies' miRNA TaqMan procedure [42] with a starting concentration for all samples of 30 ng/μl. Briefly, the procedure involved a reverse transcription step, followed by a 12-cycle pre-amplification step, and then subsequently real-time PCR (40 cycles).

Further information about the miRNA TaqMan assays are detailed in Table 19.

The raw signal intensities from the qPCR traces for each TaqMan miRNA assay were statistically analysed using methodologies known in the art. For the plasma samples, the resulting P-values and log fold changes are shown in Table 20, with a P-value <0.05 and/or a log fold change +/−0.585 being considered statistically significant. The differential expression profiles of the plasma miRNA markers were compared to the differential expression profiles of the miRNA markers previously identified in fresh PC tissue. The data in Table 20 demonstrate that there is good concordance in the miRNA expression profiles of PC plasma samples when compared to fresh PC tissues. This subset of miRNAs, derived from PC plasma, are also statistically significant for determining aggressive PC from all other sample types (i.e. indolent PC and normal), which again correlates with the fresh PC tissue data.

For the serum samples, the resulting P-values and log fold changes are shown in Table 21, with a P-value <0.05 and/or a log fold change +/−0.585 being considered statistically significant. The differential expression profiles of the serum miRNA markers were compared to the differential expression profiles of the miRNA markers previously identified in fresh PC tissue. The data in Table 21 demonstrate that there is good concordance in the miRNA expression profile of PC serum samples when compared to fresh PC tissues. This subset of miRNAs, derived from PC serum, is also statistically significant for determining PC from non-PC samples, which again correlates with the fresh PC tissue data. The inventors have therefore identified miRNA biomarkers that can be used in panels to provide a ‘molecular signature’ to successfully distinguish PC from non-PC, as well as aggressive PC from indolent PC, with a high degree of sensitivity and specificity, from various types of samples: tissues, plasma and serum samples.

Biomarkers from Plasma Samples

Table 25 provides analysis of the differential expression levels of the miRNAs of the invention in PC plasma samples compared to non-PC (BPH) plasma sample. The same sample set as the plasma experiment described above is used.

Table 26 provides analysis of the differential expression levels of the miRNAs of the invention in PC serum samples compared to non-PC (BPH) serum sample. The same sample set as the serum experiment described above is used.

Table shows plasma data with metrics (sensitivity and specificity scores, as well as area under the curve [AUC] scores) for two data sets: May 2013 (first data set) and October 2013 (second data set). The May 2013 data set used balanced sample numbers of Control, Gleason 6 and Gleason 8 samples to create a list of significant markers for both log fold change (≧0.585) and statistical significance (p-value, ≧0.05); using this data a statistical algorithm was trained. The algorithm was then tested on the subsequent data set (October 2013) to see if the data could be ‘called’ correctly. Therefore, the data, and ultimately the list of panel markers, is ordered by the AUC value for October 2013. The list of panels only contains miRNA markers that were significant between the two, independent data sets.

### Biomarkers of the Invention Demonstrate “Field Effect”

The expression levels of the miRNA biomarkers listed in Table 23 were analysed in formalin-fixed paraffin-embedded (FFPE) PC samples.

The inventors also tested whether the miRNA biomarkers of the invention show a ‘field-effect’ within prostate tissue. The concept of ‘field-effect’ within cancer dates back to the early 1950s when Slaughter et al. [43] described the phenomenon of abnormal tissue surrounding the primary site of oral squamous cell carcinoma. Since then, various researchers have demonstrated cancer field-effect within a variety of different tissues and organs, and that this field-effect has been attributed, in part, to aberrant DNA methylation in various gene(s) (e.g. 44, 45, 46).

A set of nine Gleason 8 PC formalin-fixed paraffin-embedded (FFPE) samples; eleven Gleason 6 PC FFPE samples; and ten bladder cancer FFPE samples (negative for PC) were investigated.

The FFPE samples were ethically obtained from Caucasian male donors whom had undergone radical prostatectomy to remove their entire prostate due to the presence of cancer. The prostate was then clinically assessed, using histopathology, to confirm their disease status. The PC samples used herein were either defined as Gleason 8 (aggressive) or Gleason 6 (indolent). The FFPE samples defined as “PC negative” [bladder cancer] were derived from prostate removed from the patient due to the presence of bladder cancer. However, histopathological analysis confirmed the absence of PC from these prostates.

All the FFPE samples used herein were histopathologically sectioned and stained, using hematoxylin and eosin stain, according to methodologies well known in the art. The stained sections were used to identify areas of aggressive/indolent PC (dependent on the patient in question) as well as areas of non-cancerous tissue; all areas being situated in the peripheral, glandular regions of the prostate. From any given FFPE section, five areas were marked up for subsequent macro-dissection: Area ‘A’ was the primary cancer region; areas B-D were either a secondary cancer region (with a lower Gleason score, compared to the primary cancer foci) or a non-cancerous region (dependent on the patient in question); and area ‘E’ was exclusively a non-cancerous region.

Once suitable areas had been determined, adjacent slices were taken, the areas macro-dissected, and the FFPE samples processed to extract total RNA, including the small RNA fraction (<20 nt), using standard column filtration methodologies, which are well known in the art. FFPE samples from all three cohorts were analysed using the Life Technologies' miRNA TaqMan procedure (manual part number 4465407, revision date 30 Mar. 2012 (Rev. B)) with a starting concentration for all samples of 50 ng/μl. Briefly, the procedure involved a reverse transcription step, followed by a 12-cycle pre-amplification step, and then subsequently the real-time PCR reaction (40 cycles).

The raw signal intensities from the qPCR traces for each TaqMan miRNA assay were normalised and statistically analysed using methodologies known in the art. Normalisation of the data could include, but is not limited to, the use of normaliser miRNAs. The normaliser miRNAs would have non-differential expression profiles in the same sample type.

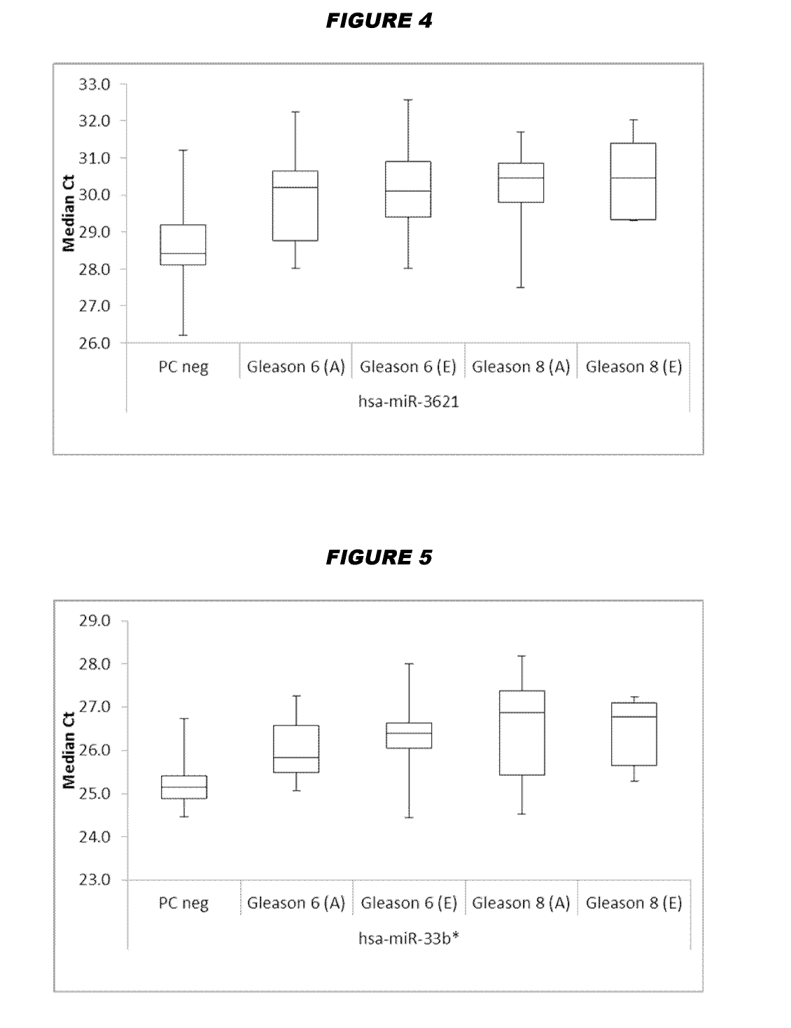

The resulting P-values and log fold changes were determined, with a P value <0.05 and a log fold change +/−0.585 being considered statistically significant. Examples of the comparison of the differential expression profile of the FFPE miRNA markers for ‘Gleason 6 (A) vs PC negative’; ‘Gleason 6 (A) vs Gleason 6 (E)’; ‘Gleason 8 (A) vs PC negative’; ‘Gleason 8 (A) vs Gleason 8 (E)’; ‘Gleason 8 (A) vs Gleason 6 (A)’; ‘Gleason 6 (E) vs PC negative’; and ‘Gleason 8 (E) vs PC negative’ for the various biomarkers are shown in FIGS. 4-11.

Referring to FIG. 4, hsa-miR-3621 shows significant differential expression between Gleason 6 (A) samples vs PC negative samples; and Gleason 8 (A) samples vs PC negative samples. This demonstrates that hsa-miR-3621 can significantly stratify PC from PC negative samples. Furthermore, there is a significant differential expression of hsa-miR-3621 between Gleason 6 (E) samples and PC negative samples; and Gleason 8 (E) samples and PC negative samples. Additionally, hsa-miR-3621 shows non-significant expression between Gleason 6 (A) samples vs Gleason 6 (E) samples; and Gleason 8 (A) samples vs Gleason 8 (E) samples. This demonstrates a hsa-miR-3621-based field-effect suggesting that any part of the prostate can be sampled (e.g. during a biopsy procedure) and the expression profile can stratify PC from PC negative samples.

Referring to FIG. 5, hsa-miR-33b* shows significant differential expression between Gleason 6 (A) samples and PC negative samples; and Gleason 8 (A) samples and PC negative samples. This demonstrates that hsa-miR-33b* can significantly stratify PC from PC negative samples. Furthermore, there is a significant differential expression of hsa-miR-33b* between Gleason 6 (E) samples vs PC negative samples; and Gleason 8 (E) samples vs PC negative samples. Additionally, hsa-miR-33b* shows non-significant expression between Gleason 6 (A) samples vs Gleason 6 (E) samples; and Gleason 8 (A) samples vs Gleason 8 (E) samples. This demonstrates a hsa-miR 33b*-based field-effect suggesting that any part of the prostate can be sampled (e.g. during a biopsy procedure) and the expression profile can stratify PC from PC negative samples.

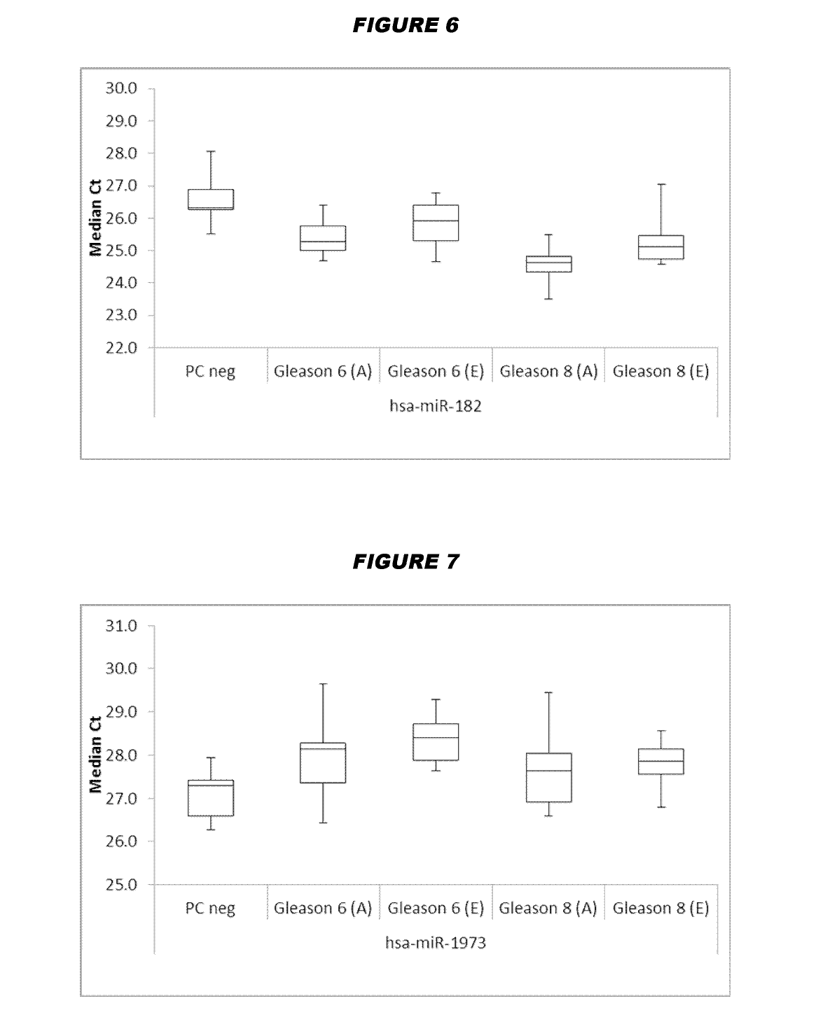

Referring to FIG. 6, hsa-miR-182 shows significant differential expression between Gleason 6 (A) samples vs PC negative samples; and Gleason 8 (A) samples vs PC negative samples. This demonstrates that hsa-miR-182 can significantly stratify PC from PC negative samples. Additionally, there is a significant differential expression of hsa-miR-182 between Gleason 8 (A) samples vs Gleason 6 (A) samples, thus demonstrating that hsa-miR-182 can significantly stratify aggressive PC from indolent PC. Furthermore, there is a significant differential expression of hsa-miR-182 between Gleason 6 (E) samples vs PC negative samples; and Gleason 8 (E) samples vs PC negative samples. In addition, hsa-miR-182 shows non-significant expression between Gleason 6 (A) samples vs Gleason 6 (E) samples; and Gleason 8 (A) samples vs Gleason 8 (E) samples. This demonstrates a hsa-miR-182-based field-effect suggesting that any part of the prostate can be sampled (e.g. during a biopsy procedure) and the expression profile can stratify PC from PC negative samples, as well as aggressive PC from indolent PC.

Referring to FIG. 7, hsa-miR-1973 shows significant differential expression between Gleason 6 (A) samples vs PC negative samples, thus demonstrating that hsa-miR-1973 can significantly stratify indolent PC from PC negative samples. Furthermore, there is a significant differential expression of miR-1973 between Gleason 6 (E) samples vs PC negative samples. In addition, hsa-miR-1973 shows non-significant expression between Gleason 6 (A) samples vs Gleason 6 (E) samples. This demonstrates a hsa-miR-1973-based field-effect suggesting that any part of the prostate can be sampled (e.g. during a biopsy procedure) and the expression profile can stratify indolent PC from PC negative samples.

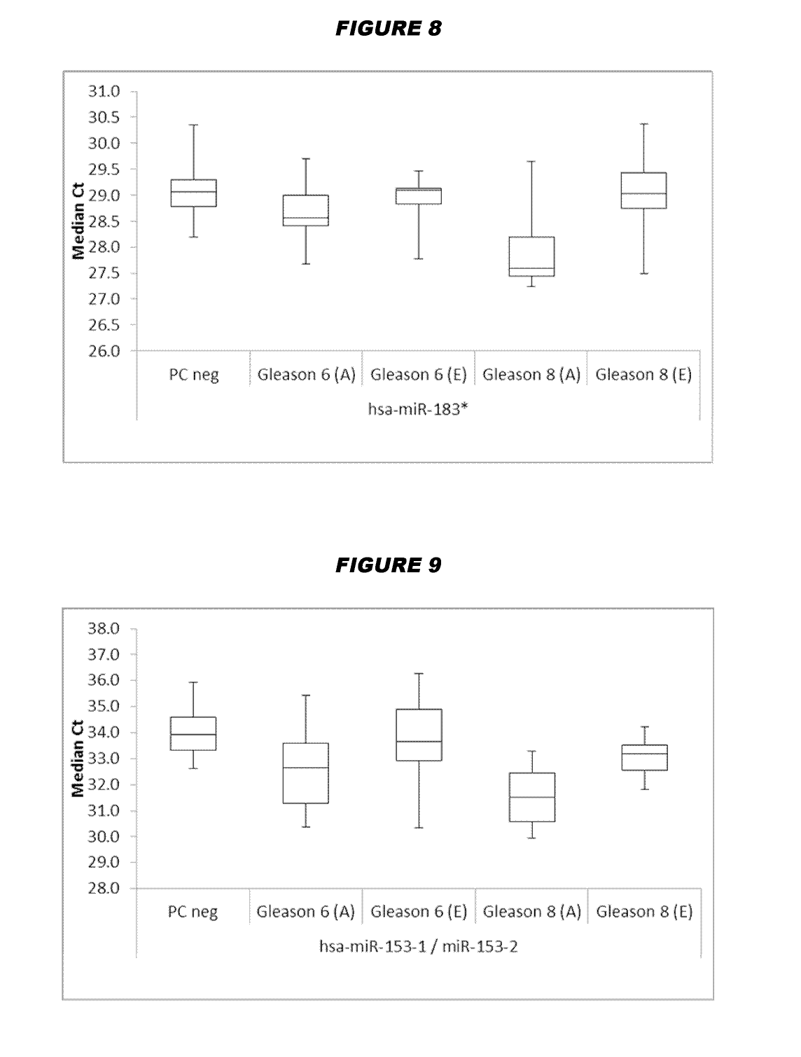

Referring to FIG. 8, hsa-miR-183* shows significant differential expression between Gleason 8 (A) samples vs PC negative samples. This demonstrates that hsa-miR-183* can significantly stratify aggressive PC from PC negative samples. Additionally, there is a non-significant expression of hsa-miR-183* between Gleason 8 (A) samples vs Gleason 8 (E) samples. This demonstrates a hsa-miR 183*-based field-effect suggesting that any part of the prostate can be sampled (e.g. during a biopsy procedure) to determine the absence of aggressive PC within the organ.

Referring to FIG. 9, hsa-miR-153-1/hsa-miR-153-2 shows significant differential expression between Gleason 6 (A) samples vs PC negative samples; and Gleason 8 (A) samples vs PC negative samples. This demonstrates that hsa-miR-153-1/hsa-miR-153-2 can significantly stratify PC from PC negative samples. Additionally, there is a significant differential expression of hsa-miR-153-1/hsa-miR-153-2 between Gleason 8 (A) samples vs Gleason 8 (E) samples, thus demonstrating a potential aggressive marker, but that the biopsy procedure would need to sample directly from the cancerous foci.

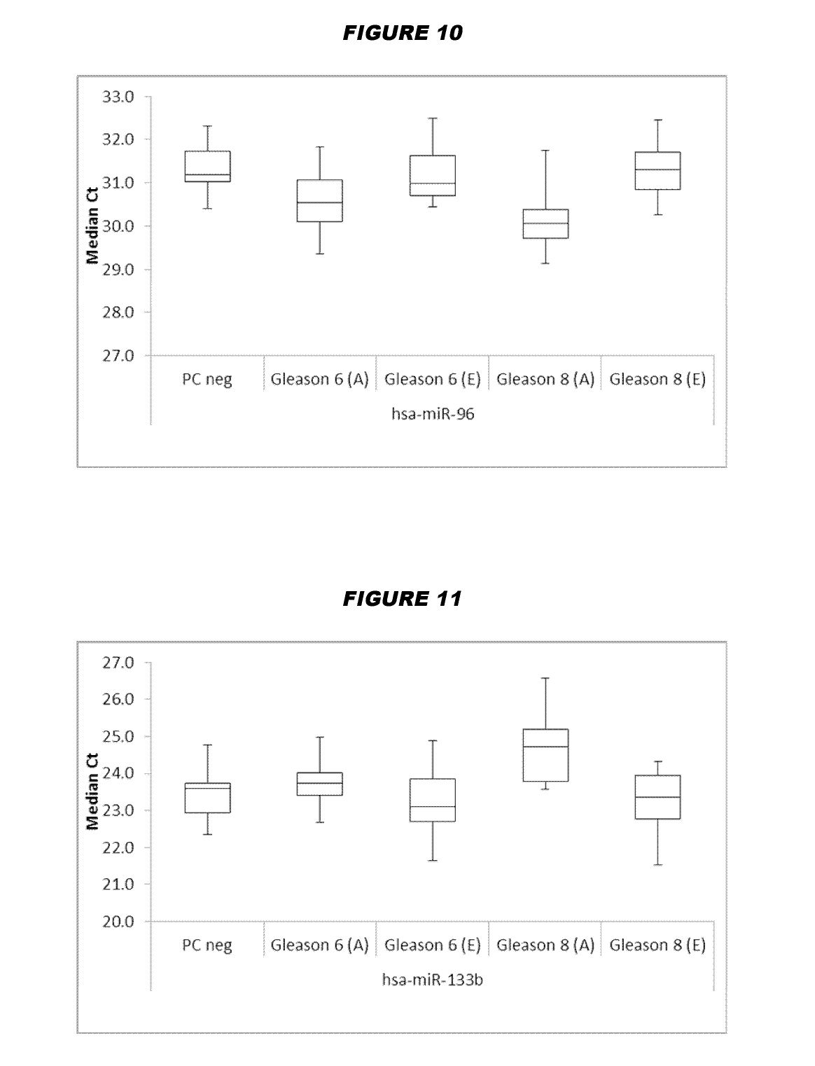

Referring to FIG. 10, hsa-miR-96 shows significant differential expression between Gleason 6 (A) samples vs PC negative samples; and Gleason 8 (A) samples vs PC negative samples, thus demonstrating that hsa-miR-96 can significantly stratify PC from PC negative samples. Additionally, there is a significant differential expression of hsa-miR-96 between Gleason 8 (A) samples vs Gleason 8 (E) samples, thus demonstrating a potential aggressive marker, but that the biopsy procedure would need to sample directly from the cancerous foci.

Referring to FIG. 11, hsa-miR-133b shows significant differential expression between Gleason 8 (A) samples vs PC negative samples; and Gleason 8 (A) samples vs Gleason 6 (A) samples. This demonstrates that hsa-miR-133b can significantly stratify aggressive PC from PC negative samples, as well as stratifying aggressive PC from indolent PC. Additionally, there is a significant differential expression of hsa-miR-133b between Gleason 8 (A) samples vs Gleason 8 (E) samples, thus demonstrating a potential aggressive marker, but that the biopsy procedure would need to sample directly from the cancerous foci.

hsa-miR-1-1/hsa-miR-1-2 and hsa-miR-99b* both show significant differential expression between Gleason 8 (A) samples vs Gleason 8 (E) samples, thus demonstrating that they are potential aggressive markers.

hsa-miR-141 shows significant differential expression between Gleason 8 (A) samples vs PC negative samples, demonstrating that hsa-miR-141 can significantly stratify aggressive PC from PC negative samples.

hsa-miR-183 shows significant differential expression between Gleason 6 (A) samples vs PC negative samples; and Gleason 8 (A) samples vs PC negative samples. This demonstrates that hsa-miR-183 can significantly stratify PC from PC negative samples. Additionally, hsa-miR-183 demonstrates a significant differential expression between Gleason 8 (A) samples vs Gleason 8 (E) samples, thus demonstrating a potential aggressive marker, but that the biopsy procedure would need to sample directly from the cancerous foci. Additionally, hsa-miR-183 shows significant differential expression between Gleason 8 (A) samples vs Gleason 6 (A) samples, thus demonstrating that hsa-miR-183 can significantly stratify aggressive PC from indolent PC.

hsa-miR-375 shows significant differential expression between Gleason 6 (A) samples vs PC negative samples; and Gleason 8 (A) samples vs PC negative samples. This demonstrates that hsa-miR-375 can significantly stratify PC from PC negative samples

hsa-miR-494 shows significant differential expression between Gleason 6 (E) samples vs PC negative samples, suggesting a hsa-miR-494-based field-effect suggesting that any part of the prostate can be sampled (e.g. during a biopsy procedure) to determine the absence of aggressive PC within the organ.

hsa-miR-582 and hsa-miR-1291 both show significant differential expression between Gleason 8 (E) samples vs PC negative samples, suggesting a hsa-miR-582-based field-effect suggesting that any part of the prostate can be sampled (e.g. during a biopsy procedure) to determine the absence of aggressive PC within the organ.

hsa-miR-133a-1/hsa-miR-133a-2 shows significant differential expression between Gleason 8 (A) samples vs Gleason 8 (E) samples, suggesting that this is a potential aggressive PC marker.

hsa-miR-182* shows significant differential expression between Gleason 8 (A) samples vs Gleason 8 (E) samples, Gleason 6 (E) samples vs PC negative samples and Gleason 8 (E) samples vs PC negative samples. This demonstrates a hsa-miR-182*-based field-effect suggesting that any part of the prostate can be sampled (e.g. during a biopsy procedure) to determine the absence of aggressive PC within the organ.

Accordingly, the inventors have identified a miRNA-based field-effect within prostate tissue that has the ability, due to the specific miRNA molecular pattern as described herein, to distinguish PC from non-PC, as well as aggressive PC from indolent PC in FFPE samples. Thus, this allows identification or predication of PC in a generalised, less targeted, sampling of the prostate during a routine biopsy procedure.

It will be understood that the invention has been described by way of example only and modifications may be made whilst remaining within the scope and spirit of the invention.

TABLE 1
Biomarkers useful with the invention
Table 1 lists biomarkers useful with the invention, for comparing samples from PC “case” and
non-PC “control”. The measured biomarker(s) can be (i) up-regulated (an increase in fold-
change, when compared to control samples) or (ii) down-regulated (a decrease in fold-change,
when compared to control samples).

| miRNA | | | | | |
|---|---|---|---|---|---|
| name⁽ⁱ⁾ | Sequence | Symbol⁽ⁱⁱ⁾ | No.⁽ⁱⁱⁱ⁾ | HGNC^{(iv)} | Expression^{(v)} |
| hsa-miR- | CGCGGGUCGGGGUCUGCAGG | MIR3621 | 1 | 38930 | UP |
| 3621 | | | | | |
| hsa-miR- | ACCAGGAGGCUGAGGCCCCU | MIR665 | 7 | 33662 | UP |
| 665 | | | | | |
| hsa-miR- | AGCUACAUUGUCUGCUGGGUUUC | MIR221 | 3 | 31601 | DOWN |
| 221 | | | | | |
| hsa-miR- | UAUGGCACUGGUAGAAUUCACU | MIR183 | 4 | 31554 | UP |
| 183 | | | | | |
| hsa-miR- | UUUGUUCGUUCGGCUCGCGUGA | MIR375 | 6 | 31868 | UP |
| 375 | | | | | |
| hsa-miR-96 | UUUGGCACUAGCACAUUUUUGCU | MIR96 | 8 | 31648 | UP |
| hsa-miR- | AGGCGGGGCGCCGCGGGACCGC | MIR663A | 10 | 32919 | UP |
| 663 | | | | | |
| hsa-miR- | UUUGGCAAUGGUAGAACUCACACU | MIR182 | 11 | 31553 | UP |
| 182 | | | | | |
| hsa-miR- | UGAAACAUACACGGGAAACCUC | MIR494 | 13 | 32084 | UP |
| 494 | | | | | |
| hsa-miR- | AAAGUUCUGAGACACUCCGACU | MIR148A | 14 | 31535 | UP |
| 148a* | | | | | |
| hsa-miR- | UGGCCCUGACUGAAGACCAGCAGU | MIR1291 | 16 | 35284 | UP |
| 1291 | | | | | |
| hsa-miR- | GACACGGGCGACAGCUGCGGCCC | MIR602 | 17 | 32858 | UP |
| 602 | | | | | |
| hsa-miR- | UGGUUCUAGACUUGCCAACUA | MIR182 | 12 | 31553 | UP |
| 182* | | | | | |
| hsa-miR- | CAGUGCCUCGGCAGUGCAGCCC | MIR33B | 19 | 32791 | UP |
| 33b* | | | | | |
| hsa-miR- | ACCGUGCAAAGGUAGCAUA | MIR1973 | 20 | 37061 | UP |
| 1973 | | | | | |
| hsa-miR- | UUGCAUAGUCACAAAAGUGAUC | MIR153-1/ | 21 | 31539/ | UP |
| 153-1/ | | MIR153-2 | | 31540 | |
| hsa-miR- | | | | | |
| 153-2 | | | | | |
| hsa-miR- | CAUCUUCCAGUACAGUGUUGGA | MIR141 | 22 | 31528 | UP |
| 141* | | | | | |
| hsa-miR- | CUCGGCGCGGGGCGCGGGCUCC | MIR1469 | 24 | 35378 | UP |
| 1469 | | | | | |
| hsa-miR- | UCCUUCAUUCCACCGGAGUCUG | MIR205 | 25 | 31583 | DOWN |
| 205 | | | | | |
| hsa-miR- | CCGUCGCCGCCACCCGAGCCG | MIR1181 | 27 | 35262 | UP |
| 1181 | | | | | |
| hsa-miR- | GCAUGUGAUGAAGCAAAUCAGU | MIR3607 | 28 | 38900 | UP |
| 3607-5p | | | | | |

### Columns (Tables 1 & 2)

(i) The “miRNA name” column gives the name of the human miRNA as provided by the specialist database, miRBase, according to version 16 (released, August 2010).

(ii) The “Symbol” column gives the gene symbol which has been approved by the Human Genome Organisation (HUGO) Gene Nomenclature Committee (HGNC). The symbol thus identifies a unique human gene. Inclusion on to HUGO is for human genes only. An additional dash-number suffix indicates pre-miRNAs that lead to identical mature miRNAs but that are located at different places in the genome.

(iii) The SEQ ID NO: for the sequence of the mature, expressed miRNA biomarker, as shown in Table 18.

(iv) The HGNC aims to give unique and meaningful names to every miRNA (and human gene). The HGNC number thus identifies a unique human gene. Inclusion on to HUGO is for human genes only.

(v) This indicates whether the miRNA is up-regulated (an increase in fold-change, e.g. at least about 1.5 fold change, when compared to control samples) or down-regulated (a decrease in fold-change, e.g. at least about 1.5 fold change, when compared to control samples. For Table 1, the control is non-PC. For Table 2, the control is indolent PC.

TABLE 2
Biomarkers useful with the invention
Table 2 lists biomarkers useful with the invention, for comparing samples from aggressive PC
“case” and indolent PC “control”. The measured biomarker(s) can be (i) up-regulated (an
increase in fold-change, when compared to control samples) or (ii) down-regulated (a decrease
in fold-change, when compared to control samples).

| miRNA name⁽ⁱ⁾ | Sequence | Symbol⁽ⁱⁱ⁾ | No.⁽ⁱⁱⁱ⁾ | HGNC^{(iv)} | Expression^{(v)} |
|---|---|---|---|---|---|
| hsa-miR-183 | UAUGGCACUGGUAGAAU | MIR183 | 4 | 31554 | UP |
| | UCACU | | | | |
| hsa-miR-96 | UUUGGCACUAGCACAUU | MIR96 | 8 | 31648 | UP |
| | UUUGCU | | | | |
| hsa-miR-182* | UGGUUCUAGACUUGCCA | MIR182 | 12 | 31553 | UP |
| | ACUA | | | | |
| hsa-miR-449a | UGGCAGUGUAUUGUUAG | MIR449A | 30 | 27645 | UP |
| | CUGGU | | | | |
| hsa-miR-133a-1/ | UUUGGUCCCCUUCAACCA | MIR133A1/ | 31 | 31517/ | DOWN |
| hsa-miR-133a-2 | GCUG | MIR133A2 | | 31518 | |
| hsa-miR-133b | UUUGGUCCCCUUCAACCA | MIR133B | 32 | 31759 | DOWN |
| | GCUA | | | | |
| hsa-miR-210 | CUGUGCGUGUGACAGCG | MIR210 | 33 | 31587 | UP |
| | GCUGA | | | | |
| hsa-miR-378a | ACUGGACUUGGAGUCAG | MIR378A | 35 | 31871 | DOWN |
| | AAGG | | | | |
| hsa-miR-99b* | CAAGCUCGUGUCUGUGG | MIR99B | 37 | 31651 | DOWN |
| | GUCCG | | | | |
| hsa-miR-1-1/ | UGGAAUGUAAAGAAGUA | MIR1-1/ | 38 | 31499/ | DOWN |
| hsa-miR-1-2 | UGUAU | MIR1-2 | | 31500 | |
| hsa-miR-429 | UAAUACUGUCUGGUAAA | MIR429 | 39 | 13784 | UP |
| | ACCGU | | | | |
| hsa-miR-139 | GGAGACGCGGCCCUGUU | MIR139 | 41 | 31526 | DOWN |
| | GGAGU | | | | |
| hsa-miR-188 | CUCCCACAUGCAGGGUU | MIR188 | 43 | 31559 | UP |
| | UGCA | | | | |
| hsa-miR-92b | UAUUGCACUCGUCCCGG | MIR92B | 45 | 32920 | DOWN |
| | CCUCC | | | | |
| hsa-miR-582 | UAACUGGUUGAACAACU | MIR582 | 47 | 32838 | DOWN |
| | GAACC | | | | |
| hsa-miR-200b | UAAUACUGCCUGGUAAU | MIR200B | 49 | 31579 | UP |
| | GAUGA | | | | |
| hsa-miR-183* | GUGAAUUACCGAAGGGC | MIR183 | 5 | 31554 | UP |
| | CAUAA | | | | |

### Panel Data (Tables 3 to 9): Disease Vs Non-Disease

Table 3-9 list biomarkers or panels of biomarkers useful with the invention, for comparing samples from PC “case” and non-PC “control”. The measured biomarker(s) can be (i) up-regulated (an increase in fold-change, when compared to control samples) or (ii) down-regulated (a decrease in fold-change, when compared to control samples).

Columns (Tables 3 to 9)
(i) The symbol for the relevant biomarker (or, for Tables 4-9, biomarkers in the panel).
(ii) S+S is the sum of the sensitivity and specificity columns. These final two columns show the sensitivity and specificity of a test based solely on the relevant biomarker (or, for Tables 4-9, panel) shown in the left-hand column when applied to the samples used in the examples.

TABLE 3

| Biomarker⁽ⁱ⁾ | S + S⁽ⁱⁱ⁾ | Sensitivity | Specificity |
|---|---|---|---|
| hsa-miR-3621 | 1.62 | 82.46% | 79.41% |
| hsa-miR-665 | 1.49 | 84.21% | 64.71% |
| hsa-miR-33b* | 1.43 | 63.16% | 79.41% |
| hsa-miR-602 | 1.41 | 82.46% | 58.82% |
| hsa-miR-3607-5p | 1.36 | 85.96% | 50.00% |
| hsa-miR-205 | 1.34 | 66.67% | 67.65% |
| hsa-miR-1973 | 1.33 | 73.68% | 58.82% |
| hsa-miR-663 | 1.32 | 82.46% | 50.00% |
| hsa-miR-1469 | 1.31 | 45.61% | 85.29% |
| hsa-miR-183 | 1.31 | 89.47% | 41.18% |

TABLE 4

| Panel | S + S | Sensitivity | Specificity |
|---|---|---|---|
| hsa-miR-3621 + hsa-miR-205 | 1.71 | 82.46% | 88% |
| hsa-miR-3621 + hsa-miR-3607-5p | 1.71 | 82.46% | 88.24% |
| hsa-miR-3621 + hsa-miR-665 | 1.68 | 82.46% | 85.29% |
| hsa-miR-665 + hsa-miR-205 | 1.67 | 75.44% | 91.18% |
| hsa-miR-3621 + hsa-miR-1469 | 1.63 | 68.42% | 94.12% |
| hsa-miR-3621 + hsa-miR-33b* | 1.61 | 78.95% | 82.35% |
| hsa-miR-3621 + hsa-miR-1181 | 1.61 | 78.95% | 82.35% |
| hsa-miR-3621 + hsa-miR-182* | 1.61 | 96.49% | 65% |
| hsa-miR-183 + hsa-miR-205 | 1.60 | 89.47% | 70.59% |
| hsa-miR-3621 + hsa-miR-602 | 1.57 | 80.70% | 76.47% |

TABLE 5

| Panel | S + S | Sensitivity | Specificity |
|---|---|---|---|
| hsa-miR-3621 + hsa-miR-1469 + | 1.78 | 80.70% | 97.06% |
| hsa-miR-205 | | | |
| hsa-miR-3621 + hsa-miR-665 + | 1.76 | 78.95% | 97.06% |
| hsa-miR-1469 | | | |
| hsa-miR-183 + hsa-miR-1469 + | 1.75 | 84.21% | 91.18% |
| hsa-miR-205 | | | |
| hsa-miR-3621 + hsa-miR-205 + | 1.75 | 80.70% | 94.12% |
| hsa-miR-1181 | | | |
| hsa-miR-3621 + hsa-miR-183 + | 1.73 | 87.72% | 85.29% |
| hsa-miR-205 | | | |
| hsa-miR-3621 + hsa-miR-1181 + | 1.72 | 84.21% | 88.24% |
| hsa-miR-3607-5p | | | |
| hsa-miR-3621 + hsa-miR-602 + | 1.72 | 80.70% | 91.18% |
| hsa-miR-205 | | | |
| hsa-miR-3621 + hsa-miR-33b* + | 1.72 | 80.70% | 91.18% |
| hsa-miR-205 | | | |
| hsa-miR-3621 + hsa-miR-602 + | 1.71 | 85.96% | 85.29% |
| hsa-miR-3607-5p | | | |
| hsa-miR-3621 + hsa-miR-1469 + | 1.71 | 82.46% | 88.24% |
| hsa-miR-3607-5p | | | |

TABLE 6

| Panel | S + S | Sensitivity | Specificity |
|---|---|---|---|
| hsa-miR-3621 + hsa-miR-183 + | 1.80 | 85.96% | 94.12% |
| hsa-miR-1469 + hsa-miR-205 | | | |
| hsa-miR-3621 + hsa-miR-183 + | 1.78 | 84.21% | 94.12% |
| hsa-miR-205 + hsa-miR-3607-5p | | | |
| hsa-miR-183 + hsa-miR-33b* + | 1.78 | 84.21% | 94% |
| hsa-miR-1469 + hsa-miR-205 | | | |
| hsa-miR-183 + hsa-miR-1469 + | 1.78 | 84.21% | 94.12% |
| hsa-miR-205 + hsa-miR-3607-5p | | | |
| hsa-miR-183 + hsa-miR-665 + | 1.77 | 85.96% | 91.18% |
| hsa-miR-1469 + hsa-miR-205 | | | |
| hsa-miR-183 + hsa-miR-602 + | 1.77 | 85.96% | 91.18% |
| hsa-miR-1469 + hsa-miR-205 | | | |
| hsa-miR-3621 + hsa-miR-663 + | 1.76 | 78.95% | 97.06% |
| hsa-miR-33b* + hsa-miR-205 | | | |
| hsa-miR-3621 + hsa-miR-602 + | 1.76 | 78.95% | 97.06% |
| hsa-miR-205 + hsa-miR-3607-5p | | | |
| hsa-miR-3621 + hsa-miR-33b* + | 1.76 | 78.95% | 97.06% |
| hsa-miR-205 + hsa-miR-1181 | | | |
| hsa-miR-183 + hsa-miR-663 + | 1.75 | 84.21% | 91.18% |
| hsa-miR-1469 + hsa-miR-205 | | | |

TABLE 7

| Panel | S + S | Sensitivity | Specificity |
|---|---|---|---|
| hsa-miR-183 + hsa-miR-665 + hsa-miR- | 1.83 | 85.96% | 97.06% |
| 1469 + hsa-miR-205 + hsa-miR-3607-5p | | | |
| hsa-miR-183 + hsa-miR-182* + hsa-miR- | 1.81 | 84.21% | 97.06% |
| 1469 + hsa-miR-205 + hsa-miR-3607-5p | | | |
| hsa-miR-3621 + hsa-miR-183 + hsa-miR- | 1.80 | 85.96% | 94.12% |
| 665 + hsa-miR-1469 + hsa-miR-205 | | | |
| hsa-miR-3621 + hsa-miR-183 + hsa-miR- | 1.80 | 85.96% | 94.12% |
| 663 + hsa-miR-1469 + hsa-miR-205 | | | |
| hsa-miR-3621 + hsa-miR-183 + hsa-miR- | 1.80 | 85.96% | 94.12% |
| 1291 + hsa-miR-1469 + hsa-miR-205 | | | |
| hsa-miR-3621 + hsa-miR-183 + hsa-miR- | 1.80 | 85.96% | 94.12% |
| 602 + hsa-miR-1469 + hsa-miR-205 | | | |
| hsa-miR-3621 + hsa-miR-183 + hsa-miR- | 1.80 | 85.96% | 94.12% |
| 182* + hsa-miR-1469 + hsa-miR-205 | | | |
| hsa-miR-3621 + hsa-miR-183 + hsa-miR- | 1.80 | 85.96% | 94.12% |
| 33b* + hsa-miR-1469 + hsa-miR-205 | | | |
| hsa-miR-3621 + hsa-miR-183 + hsa-miR- | 1.80 | 85.96% | 94.12% |
| 1469 + hsa-miR-205 + hsa-miR-1181 | | | |
| hsa-miR-183 + hsa-miR-665 + hsa-miR- | 1.80 | 85.96% | 94.12% |
| 602 + hsa-miR-1469 + hsa-miR-205 | | | |

TABLE 8

| Panel | S + S | Sensitivity | Specificity |
|---|---|---|---|
| hsa-miR-183 + hsa-miR-665 + hsa-miR- | 1.83 | 85.96% | 97.06% |
| 663 + hsa-miR-1469 + hsa-miR- | | | |
| 205 + hsa-miR-3607-5p | | | |
| hsa-miR-183 + hsa-miR-665 + hsa-miR- | 1.83 | 85.96% | 97.06% |
| 182* + hsa-miR-1469 + hsa-miR- | | | |
| 205 + hsa-miR-3607-5p | | | |
| hsa-miR-665 + hsa-miR-182 + hsa-miR- | 1.82 | 87.72% | 94.12% |
| 1291 + hsa-miR-1469 + hsa-miR- | | | |
| 205 + hsa-miR-3607-5p | | | |
| hsa-miR-3621 + hsa-miR-182 + hsa-miR- | 1.81 | 84.21% | 97.06% |
| 494 + hsa-miR-1973 + hsa-miR- | | | |
| 1469 + hsa-miR-205 | | | |
| hsa-miR-183 + hsa-miR-665 + hsa-miR- | 1.81 | 84.21% | 97% |
| 1291 + hsa-miR-1469 + hsa-miR- | | | |
| 205 + hsa-miR-3607-5p | | | |
| hsa-miR-183 + hsa-miR-663 + hsa-miR- | 1.81 | 84.21% | 97.06% |
| 1291 + hsa-miR-33b* + hsa-miR- | | | |
| 205 + hsa-miR-3607-5p | | | |
| hsa-miR-183 + hsa-miR-663 + hsa-miR- | 1.81 | 84.21% | 97.06% |
| 182* + hsa-miR-1469 + hsa-miR- | | | |
| 205 + hsa-miR-3607-5p | | | |
| hsa-miR-183 + hsa-miR-182* + hsa-miR- | 1.81 | 84.21% | 97.06% |
| 33b* + hsa-miR-1469 + hsa-miR- | | | |
| 205 + hsa-miR-3607-5p | | | |
| hsa-miR-183 + hsa-miR-182* + hsa-miR- | 1.81 | 84.21% | 97.06% |
| 1973 + hsa-miR-1469 + hsa-miR- | | | |
| 205 + hsa-miR-3607-5p | | | |
| hsa-miR-182 + hsa-miR-1291 + hsa-miR- | 1.81 | 84.21% | 97.06% |
| 1973 + hsa-miR-1469 + hsa-miR- | | | |
| 205 + hsa-miR-3607-5p | | | |

TABLE 9

| Panel | S + S | Sensitivity | Specificity |
|---|---|---|---|
| hsa-miR-3621 + hsa-miR-183 + hsa- | 1.83 | 85.96% | 97.06% |
| miR-665 + hsa-miR-663 + hsa-miR- | | | |
| 182 + hsa-miR-1469 + hsa-miR-205 | | | |
| hsa-miR-3621 + hsa-miR-183 + hsa- | 1.83 | 85.96% | 97.06% |
| miR-663 + hsa-miR-182 + hsa-miR- | | | |
| 602 + hsa-miR-1469 + hsa-miR-205 | | | |
| hsa-miR-3621 + hsa-miR-1291 + hsa- | 1.83 | 85.96% | 97.06% |
| miR-602 + hsa-miR-1973 + hsa-miR- | | | |
| 1469 + hsa-miR-205 + hsa-miR-1181 | | | |
| hsa-miR-183 + hsa-miR-665 + hsa-miR- | 1.83 | 85.96% | 97.06% |
| 663 + hsa-miR-1291 + hsa-miR- | | | |
| 1469 + hsa-miR-205 + hsa-miR-3607-5p | | | |
| hsa-miR-183 + hsa-miR-665 + hsa-miR- | 1.83 | 85.96% | 97.06% |
| 663 + hsa-miR-182* + hsa-miR- | | | |
| 1469 + hsa-miR-205 + hsa-miR-3607-5p | | | |
| hsa-miR-183 + hsa-miR-665 + hsa-miR- | 1.83 | 85.96% | 97.06% |
| 182 + hsa-miR-1291 + hsa-miR- | | | |
| 1469 + hsa-miR-205 + hsa-miR-3607-5p | | | |
| hsa-miR-183 + hsa-miR-182 + hsa-miR- | 1.83 | 85.96% | 97.06% |
| 1291 + hsa-miR-182* + hsa-miR- | | | |
| 1469 + hsa-miR-205 + hsa-miR-3607-5p | | | |
| hsa-miR-663 + hsa-miR-182 + hsa-miR- | 1.83 | 85.96% | 97.06% |
| 1291 + hsa-miR-1973 + hsa-miR- | | | |
| 1469 + hsa-miR-205 + hsa-miR-3607-5p | | | |
| hsa-miR-182 + hsa-miR-1291 + hsa- | 1.83 | 85.96% | 97.06% |
| miR-33b* + hsa-miR-1973 + hsa-miR- | | | |
| 1469 + hsa-miR-205 + hsa-miR-3607-5p | | | |
| hsa-miR-182 + hsa-miR-1291 + hsa- | 1.83 | 85.96% | 97.06% |
| miR-1973 + hsa-miR-1469 + hsa-miR- | | | |
| 205 + hsa-miR-1181 + hsa-miR-3607-5p | | | |

### Panel Data (Tables 10 to 16): Aggressive Vs Indolent

Table 10-16 list biomarkers or panels of biomarkers useful with the invention, for comparing samples from aggressive PC “case” and indolent PC “control”. The measured biomarker(s) can be (i) up-regulated (an increase in fold-change, when compared to control samples) or (ii) down-regulated (a decrease in fold-change, when compared to control samples).

Columns (Tables 10 to 16)
(i) The symbol for the relevant biomarker (or, for Tables 11-16, biomarkers in the panel).
(ii) S+S is the sum of the sensitivity and specificity columns. These final two columns show the sensitivity and specificity of a test based solely on the relevant biomarker (or, for Tables 11-16, panel) shown in the left-hand column when applied to the samples used in the examples.

TABLE 10

| Biomarker⁽ⁱ⁾ | S + S⁽ⁱⁱ⁾ | Sensitivity | Specificity |
|---|---|---|---|
| hsa-miR-99b* | 1.24 | 82.35% | 41.18% |
| hsa-miR-210 | 1.14 | 64.71% | 49.02% |
| hsa-miR-200b | 1.12 | 70.59% | 41.18% |
| hsa-miR-183 | 1.06 | 88.24% | 17.65% |
| hsa-miR-92b | 1.06 | 88.24% | 17.65% |
| hsa-miR-183* | 1.06 | 94.12% | 11.76% |
| hsa-miR-449a | 1.02 | 94.12% | 7.84% |
| hsa-miR-133b | 0.96 | 94.12% | 1.96% |
| hsa-miR-182* | 0.96 | 94.12% | 1.96% |
| hsa-miR-139 | 0.96 | 94.12% | 1.96% |

TABLE 11

| Panel | S + S | Sensitivity | Specificity |
|---|---|---|---|
| hsa-miR-99b* + hsa-miR-200b | 1.41 | 94.12% | 47.06% |
| hsa-miR-210 + hsa-miR-99b* | 1.35 | 94% | 41.18% |
| hsa-miR-183 + hsa-miR-99b* | 1.31 | 94.12% | 37.25% |
| hsa-miR-99b* + hsa-miR-183* | 1.31 | 94.12% | 37.25% |
| hsa-miR-182* + hsa-miR-99b* | 1.29 | 94.12% | 35.29% |
| hsa-miR-99b* + hsa-miR-139 | 1.29 | 94.12% | 35.29% |
| hsa-miR-99b* + hsa-miR-96 | 1.29 | 88.24% | 41.18% |
| hsa-miR-99b* + hsa-miR-582-3p | 1.29 | 88.24% | 41.18% |
| hsa-miR-99b* + hsa-miR-1 | 1.27 | 94.12% | 33.33% |
| hsa-miR-99b* + hsa-miR-429 | 1.27 | 94.12% | 33.33% |

TABLE 12

| Panel | S + S | Sensitivity | Specificity |
|---|---|---|---|
| hsa-miR-182* + hsa-miR-99b* + hsa- | 1.41 | 94.12% | 47.06% |
| miR-200b | | | |
| hsa-miR-99b* + hsa-miR-429 + hsa- | 1.41 | 94.12% | 47.06% |
| miR-96 | | | |
| hsa-miR-210 + hsa-miR-99b* + hsa- | 1.39 | 82.35% | 56.86% |
| miR-96 | | | |
| hsa-miR-99b* + hsa-miR-582-3p + hsa- | 1.39 | 94.12% | 45.10% |
| miR-200b | | | |
| hsa-miR-210 + hsa-miR-99b* + hsa-miR- | 1.37 | 94.12% | 43.14% |
| 582-3p | | | |
| hsa-miR-182* + hsa-miR-99b* + hsa- | 1.37 | 94.12% | 43.14% |
| miR-183* | | | |
| hsa-miR-183 + hsa-miR-99b* + hsa- | 1.37 | 94.12% | 43.14% |
| miR-200b | | | |
| hsa-miR-183 + hsa-miR-139 + hsa- | 1.37 | 76.47% | 60.78% |
| miR-96 | | | |
| hsa-miR-99b* + hsa-miR-96 + hsa- | 1.37 | 82.35% | 54.90% |
| miR-200b | | | |
| hsa-miR-99b* + hsa-miR-92b + hsa- | 1.37 | 94.12% | 43.14% |
| miR-200b | | | |

TABLE 13

| Panel | S + S | Sensitivity | Specificity |
|---|---|---|---|
| hsa-miR-210 + hsa-miR-99b* + hsa-miR- | 1.53 | 88.24% | 64.71% |
| 429 + hsa-miR-96 | | | |
| hsa-miR-210 + hsa-miR-99b* + hsa-miR- | 1.53 | 88.24% | 64.71% |
| 139 + hsa-miR-96 | | | |
| hsa-miR-210 + hsa-miR-99b* + hsa- | 1.51 | 76.47% | 74.51% |
| miR-96 + hsa-miR-200b | | | |
| hsa-miR-449a + hsa-miR-99b* + hsa- | 1.49 | 88.24% | 60.78% |
| miR-96 + hsa-miR-200b | | | |
| hsa-miR-133a + hsa-miR-210 + hsa-miR- | 1.49 | 94.12% | 54.90% |
| 99b* + hsa-miR-96 | | | |
| hsa-miR-133b + hsa-miR-210 + hsa-miR- | 1.49 | 94.12% | 54.90% |
| 99b* + hsa-miR-96 | | | |
| hsa-miR-210 + hsa-miR-99b* + hsa-miR- | 1.49 | 88.24% | 60.78% |
| 96 + hsa-miR-188-3p | | | |
| hsa-miR-182* + hsa-miR-99b* + hsa- | 1.49 | 94.12% | 54.90% |
| miR-429 + hsa-miR-96 | | | |
| hsa-miR-99b* + hsa-miR-96 + hsa-miR- | 1.49 | 82.35% | 66.67% |
| 188-3p + hsa-miR-200b | | | |
| hsa-miR-210 + hsa-miR-182* + hsa-miR- | 1.47 | 70.59% | 76.47% |
| 139 + hsa-miR-96 | | | |

TABLE 14

| Panel | S + S | Sensitivity | Specificity |
|---|---|---|---|
| hsa-miR-449a + hsa-miR-99b* + hsa- | 1.63 | 94.12% | 68.63% |
| miR-96 + hsa-miR-188-3p + hsa- | | | |
| miR-200b | | | |
| hsa-miR-133a + hsa-miR-210 + hsa-miR- | 1.59 | 88.24% | 70.59% |
| 99b* + hsa-miR-96 + hsa-miR-200b | | | |
| hsa-miR-210 + hsa-miR-378 + hsa-miR- | 1.59 | 94.12% | 64.71% |
| 99b* + hsa-miR-96 + hsa-miR-200b | | | |
| hsa-miR-210 + hsa-miR-99b* + hsa- | 1.59 | 94.12% | 64.71% |
| miR-1 + hsa-miR-429 + hsa-miR-96 | | | |
| hsa-miR-210 + hsa-miR-99b* + hsa- | 1.59 | 82.35% | 76.47% |
| miR-1 + hsa-miR-96 + hsa-miR-200b | | | |
| hsa-miR-210 + hsa-miR-99b* + hsa-miR- | 1.59 | 70.59% | 88.24% |
| 139 + hsa-miR-96 + hsa-miR-183* | | | |
| hsa-miR-210 + hsa-miR-378 + hsa-miR- | 1.57 | 88.24% | 68.63% |
| 99b* + hsa-miR-96 + hsa-miR-183* | | | |
| hsa-miR-210 + hsa-miR-183 + hsa-miR- | 1.57 | 70.59% | 86.27% |
| 139 + hsa-miR-96 + hsa-miR-188-3p | | | |
| hsa-miR-99b* + hsa-miR-1 + hsa- | 1.57 | 94.12% | 62.75% |
| miR-429 + hsa-miR-96 + hsa-miR-183* | | | |
| hsa-miR-449a + hsa-miR-99b* + hsa- | 1.55 | 88.24% | 66.67% |
| miR-1 + hsa-miR-96 + hsa-miR-200b | | | |

TABLE 15

| | | Sensi- | Spe- |
|---|---|---|---|
| Panel | S + S | tivity | cificity |
| hsa-miR-210 + hsa-miR-378 + hsa-miR- | 1.69 | 88.24% | 80.39% |
| 99b* + hsa-miR-139 + hsa-miR-96 + hsa-miR- | | | |
| 183* | | | |
| hsa-miR-449a + hsa-miR-183 + hsa-miR- | 1.67 | 94.12% | 72.55% |
| 99b* + hsa-miR-96 + hsa-miR-188-3p + hsa- | | | |
| miR-200b | | | |
| hsa-miR-133b + hsa-miR-210 + hsa-miR- | 1.67 | 88.24% | 78.43% |
| 99b* + hsa-miR-96 + hsa-miR-188-3p + hsa- | | | |
| miR-200b | | | |
| hsa-miR-210 + hsa-miR-378 + hsa-miR- | 1.67 | 94.12% | 72.55% |
| 99b* + hsa-miR-429 + hsa-miR-96 + hsa-miR- | | | |
| 183* | | | |
| hsa-miR-449a + hsa-miR-133b + hsa-miR- | 1.65 | 88.24% | 76.47% |
| 210 + hsa-miR-99b* + hsa-miR-96 + hsa-miR- | | | |
| 200b | | | |
| hsa-miR-449a + hsa-miR-210 + hsa-miR- | 1.65 | 94.12% | 70.59% |
| 378 + hsa-miR-99b* + hsa-miR-96 + hsa-miR- | | | |
| 200b | | | |
| hsa-miR-449a + hsa-miR-183 + hsa-miR- | 1.65 | 94.12% | 70.59% |
| 99b* + hsa-miR-429 + hsa-miR-96 + hsa-miR- | | | |
| 188-3p | | | |
| hsa-miR-449a + hsa-miR-99b* + hsa-miR- | 1.65 | 94% | 70.59% |
| 1 + hsa-miR-96 + hsa-miR-188-3p + hsa-miR- | | | |
| 200b | | | |
| hsa-miR-133a + hsa-miR-210 + hsa-miR- | 1.65 | 88% | 76.47% |
| 99b* + hsa-miR-96 + hsa-miR-188-3p + hsa- | | | |
| miR-200b | | | |
| hsa-miR-133b + hsa-miR-210 + hsa-miR- | 1.65 | 88.24% | 76.47% |
| 99b* + hsa-miR-139 + hsa-miR-96 + hsa-miR- | | | |
| 200b | | | |

TABLE 16

| | | Sensi- | Spe- |
|---|---|---|---|
| Panel | S + S | tivity | cificity |
| hsa-miR-133a + hsa-miR-210 + hsa-miR- | 1.71 | 88.24% | 82.35% |
| 99b* + hsa-miR-139 + hsa-miR-96 + hsa-miR- | | | |
| 188-3p + hsa-miR-200b | | | |
| hsa-miR-133b + hsa-miR-210 + hsa-miR- | 1.71 | 88.24% | 82.35% |
| 99b* + hsa-miR-139 + hsa-miR-96 + hsa-miR- | | | |
| 188-3p + hsa-miR-200b | | | |
| hsa-miR-210 + hsa-miR-378 + hsa-miR- | 1.71 | 94.12% | 76.47% |
| 99b* + hsa-miR-139 + hsa-miR-96 + hsa-miR- | | | |
| 200b + hsa-miR-183* | | | |
| hsa-miR-449a + hsa-miR-133a + hsa-miR- | 1.69 | 88.24% | 80.39% |
| 210 + hsa-miR-99b* + hsa-miR-96 + hsa-miR- | | | |
| 188-3p + hsa-miR-200b | | | |
| hsa-miR-449a + hsa-miR-210 + hsa-miR- | 1.69 | 88.24% | 80.39% |
| 183 + hsa-miR-99b* + hsa-miR-429 + hsa- | | | |
| miR-188-3p + hsa-miR-200b | | | |
| hsa-miR-449a + hsa-miR-182* + hsa-miR- | 1.69 | 94.12% | 74.51% |
| 183 + hsa-miR-99b* + hsa-miR-96 + hsa- | | | |
| miR-188-3p + hsa-miR-200b | | | |
| hsa-miR-133a + hsa-miR-210 + hsa-miR- | 1.69 | 88.24% | 80.39% |
| 99b* + hsa-miR-429 + hsa-miR-96 + hsa-miR- | | | |
| 188-3p + hsa-miR-200b | | | |
| hsa-miR-210 + hsa-miR-378 + hsa-miR- | 1.69 | 88.24% | 80.39% |
| 182* + hsa-miR-99b* + hsa-miR-139 + hsa- | | | |
| miR-96 + hsa-miR-200b | | | |
| hsa-miR-210 + hsa-miR-378 + hsa-miR- | 1.69 | 88.24% | 80.39% |
| 99b* + hsa-miR-1 + hsa-miR-139 + hsa-miR- | | | |
| 96 + hsa-miR-183* | | | |
| hsa-miR-210 + hsa-miR-378 + hsa-miR- | 1.69 | 88.24% | 80.39% |
| 99b* + hsa-miR-139 + hsa-miR-96 + hsa-miR- | | | |
| 188-3p + hsa-miR-200b | | | |

TABLE 17

| No.⁽ⁱ⁾ | Symbol⁽ⁱⁱ⁾ | Name⁽ⁱⁱⁱ⁾ | GI^{(iv)} | ID^{(v)} |
|---|---|---|---|---|
| 1 | MIR3621 | microRNA 3621 | 312147424 | 100500811 |
| 3 | MIR221 | microRNA 221 | 262206342 | 407006 |
| 4 | MIR183 | microRNA 183 | 262206247 | 406959 |
| 6 | MIR375 | microRNA 375 | 262206227 | 494324 |
| 7 | MIR665 | microRNA 665 | 262206150 | 100126315 |
| 8 | MIR96 | microRNA 96 | 262205747 | 407053 |
| 10 | MIR663A | microRNA 663a | 262206270 | 724033 |
| 11 | MIR182 | microRNA 182 | 262206242 | 406958 |
| 13 | MIR494 | microRNA 494 | 262205218 | 574452 |
| 14 | MIR148A | microRNA 148a | 262206160 | 406940 |
| 16 | MIR1291 | microRNA 1291 | 269847156 | 100302221 |
| 17 | MIR602 | microRNA 602 | 262206006 | 693187 |
| 12 | MIR182 | microRNA 182 | 262206242 | 406958 |
| 19 | MIR33B | microRNA 33b | 262206145 | 693120 |
| 20 | MIR1973 | microRNA 1973 | 269847660 | 100302290 |
| 21 | MIR153-1/ | microRNA 153-1/ | 262205338/ | 406944/ |
| | MIR153-2 | microRNA 153-2 | 262205343 | 406945 |
| 22 | MIR141 | microRNA 141 | 262205311 | 406933 |
| 24 | MIR1469 | microRNA 1469 | 269847566 | 100302258 |
| 25 | MIR205 | microRNA 205 | 262206281 | 406988 |
| 27 | MIR1181 | microRNA 1181 | 269847026 | 100302213 |
| 28 | MIR3607 | microRNA 3607 | 312147410 | 100500805 |
| 30 | MIR449A | microRNA 449a | 262205416 | 554213 |
| 31 | MIR133A1/ | microRNA 133a-1/ | 262205283/ | 406922/ |
| | MIR133A2 | microRNA 133a-2 | 262205288 | 406923 |
| 32 | MIR133B | microRNA 133b | 262205134 | 442890 |
| 33 | MIR210 | microRNA 210 | 262206286 | 406992 |
| 35 | MIR378A | microRNA 378a | 262206243 | 494327 |
| 37 | MIR99B | microRNA 99b | 262206116 | 407056 |
| 38 | MIR1-1/ | microRNA 1-1/ | 262205804/ | 406904/ |
| | MIR1-2 | microRNA 1-2 | 262205216 | 406905 |
| 39 | MIR429 | microRNA 429 | 262205400 | 554210 |
| 41 | MIR139 | microRNA 139 | 262206187 | 406931 |
| 43 | MIR188 | microRNA 188 | 262205439 | 406964 |
| 45 | MIR92B | microRNA 92b | 262205754 | 693235 |
| 47 | MIR582 | microRNA 582 | 262205881 | 693167 |
| 49 | MIR200B | microRNA 200b | 262206358 | 406984 |
| 5 | MIR183 | microRNA 183 | 262206247 | 406959 |

Table 17 lists all the biomarkers useful with the invention (from Table 1 and Table 2). Table 17 states the official name of the miRNA biomarkers (according to NCBI), as well as their unique GenInfo Identifier number and Entrez GeneID number.

### Columns

(i) This number is the SEQ ID NO: for the sequence of the mature, expressed miRNA biomarker, as shown in the sequence listing.

(ii) The “Symbol” column is as described for Table 1.

(iii) This name is taken from the Official Full Name provided by National Center for Biotechnology Information (NCBI). A miRNA antigen may have been referred to by one or more pseudonyms in the prior art. The invention relates to these miRNA regardless of their nomenclature.

(iv) A “GI” number, “GenInfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) it receives a new GI number. Thus the sequence associated with a given GI number is never changed.

(v) The “ID” column shows the Entrez GeneID number for the miRNA. An Entrez GeneID value is unique across all taxa.

### REFERENCE

[1] Gleason (1992) Hum Pathol 23:273-9
[2] Ostrander et al. (2006) Cancer Epidemiol Biomarkers Prev 15:1761-4
[3] Stanford et al. (2006) Prostate 66:317-25
[4] Harvey et al. (2009) BMC Urol. 9:14.
[5] Bussemakers et al. (1999) Cancer Res 59:5975-9.
[6] Hessels et al. (2003) Eur Urol 44:8-16.
[7] Groskopf et al. (2006) Clin Chem 52:1089-95.
[8] Marks et al. (2007) Urology 69:532-5.
[9] US-2008/0081339.
[10] US-2009/0226915.
[11] US-2009/0258792.
[12] Wang et al. (2009) Cancer 115(17): 3879-86
[13] Boyle & Brawley (2009) CA Cancer J Clin 59:220-4.
[14] Griffiths-Jones (2004) Nucleic Acid Res 32 (Database Issue):D109-11.
[15] Griffiths-Jones et al. (2006) Nucleic Acid Res 34 (Database Issue):D140-4.
[16] Griffiths-Jones et al. (2008) Nucleic Acid Res 36 (Database Issue):D154-8.
[17] Kozomara & Griffiths-Jones (2011) Nucleic Acid Res 39 (Database Issue):D152-7. http://www.mirbase.org/[18]
[18] Maragkakis et al. (2009) Nucleic Acid Res 37 (Web Server Issue):W273-6.
[19] Maragkakis et al. (2011) Nucleic Acid Res 39 (Web Server Issue):W145-8. http://diana.cslab.ece.ntua.gr/DianaTools/index.php?r=microtv4/index
[20] Betel et al. (2008) Nucleic Acid Res 36 (Database Issue):D149-53. http://www.microrna.org/microrna/home.do
[21] Wang & El Naqa (2008) Bioinformatics 24:325-32.
[22] Wang (2008) RNA 14:1012-7. http://mirdb.org/miRDB/index.html
[23] Grimson et al. (2007) Mol Cell 27:91-105. http://www.targetscan.org/vert_{—}50/[24]
[24] Krek et al. (2005) Nat Genet 37:495-500. http://pictar.mdc-berlin.de/cgi-bin/PicTar_vertebrate.cgi
[25] Sonn et al. (2005) Prostate Cancer Prostatic Dis 8:304-10.
[26] Pradervand et al. (2010) Biotechniques 48:219-22.
[27] Marcucci et al. J Clin Oncol. (2013) 31-17:2086
[28] WO93/22480.
[29] WO03/020415.
[30] PCT/GB2004/004390.
[31] Bolstad et al. (2003) Bioinformatics 19:185-93.
[32] Meyer et al. (2003) Neurocomputing 55:169-86.
[33] Koza (1992), Genetic Programming: On the Programming of Computers by Means of Natural Selection, MIT Press.
[34] Makinani (2005) World Acad Sci Eng Tech 7:166-9.
[35] Zhan & Shen (2004) LNCS 3314/2005.
[36] çinar et al. (2009) Expert Systems with Applications 36:6357-61.
[37] Gleave & Monia (2005) Nat Rev Cancer 5:468-79.
[38] Current Protocols in Molecular Biology (F. M. Ausubel et al., eds., 1987) Supplement 30
[39] Smith & Waterman (1981) Adv. Appl. Math. 2: 482-489.
[40] Chen et al. (2008) Cell Res 18:997-1006
[41] Mahn et al., (2011) Urology. 77(5):1265. e9-16.
[42] Manual part number 4465407, revision date 30 Mar. 2012 (Rev. B)
[43] Slaughter et al. (1953) Cancer 6: 963-8.
[44] Chai & Brown (2009) Ann Clin Lab Sci 39: 331-7
[45] Giovannucci & Ogino (2005) J Natl Cancer Inst 97: 1317-9
[46] Wasserkort et al. (2013) BMC Cancer 13: 398 [Epub]

TABLE 18

| | | | SEQ | Mature | Mature | SEQ | Mature | Mature | SEQ | |
|---|---|---|---|---|---|---|---|---|---|---|
| miRNA | Hairpin | Hairpin | ID | accession | sequence | ID | accession | sequence | ID | Chromosomal |
| name⁽ⁱ⁾ | accession⁽ⁱⁱ⁾ | sequence⁽ⁱⁱⁱ⁾ | NO | (-5p)^{(iv)} | (-5p)^{(v)} | NO | (-3p)^{(vi)} | (-3p)^{(vii)} | NO | location^{(viii)} |
| hsa- | MI0016012 | GUGAGCUGCUGGGGA | 50 | MIMAT00 | CGCGGGUC | 1 | | | | chr9:1391834 |
| miR- | | CGCGGGUCGGGGUCU | | 18002 | GGGGUCUG | | | | | 59-139183543 |
| 3621 | | GCAGGGCGGUGCGGC | | | CAGG | | | | | |
| | | AGCCGCCACCUGACG | | | | | | | | |
| | | CCGCGCCUUUGUCUG | | | | | | | | |
| | | UGUCCCACAG | | | | | | | | |
| hsa- | MI0000298 | UGAACAUCCAGGUCU | 51 | MIMAT00 | ACCUGGCA | 2 | MIMAT00 | AGCUACAU | 3 | chrX:4549052 |
| miR- | | GGGGCAUGAACCUGG | | 04568 | UACAAUGU | | 00278 | UGUCUGCU | | 9-45490638 |
| 221 | | CAUACAAUGUAGAUU | | | AGAUUU | | | GGGUUUC | | |
| | | UCUGUGUUCGUUAGG | | | | | | | | |
| | | CAACAGCUACAUUGU | | | | | | | | |
| | | CUGCUGGGUUUCAGG | | | | | | | | |
| | | CUACCUGGAAACAUG | | | | | | | | |
| | | UUCUC | | | | | | | | |
| hsa- | MI0000273 | CCGCAGAGUGUGACU | 52 | MIMAT00 | UAUGGCAC | 4 | MIMAT00 | GUGAAUUA | 5 | chr7:1292019 |
| miR- | | CCUGUUCUGUGUAUG | | 00261 | UGGUAGAA | | 04560 | CCGAAGGG | | 81-129202090 |
| 183 | | GCACUGGUAGAAUUC | | | UUCACU | | | CCAUAA | | |
| | | ACUGUGAACAGUCUC | | | | | | | | |
| | | AGUCAGUGAAUUACC | | | | | | | | |
| | | GAAGGGCCAUAAACA | | | | | | | | |
| | | GAGCAGAGACAGAUC | | | | | | | | |
| | | CACGA | | | | | | | | |
| hsa- | MI0000783 | CCCCGCGACGAGCCC | 53 | MIMAT00 | UUUGUUCG | 6 | | | | chr2:2195746 |
| miR- | | CUCGCACAAACCGGA | | 00728 | UUCGGCUC | | | | | 11-219574674 |
| 375 | | CCUGAGCGUUUUGUU | | | GCGUGA | | | | | |
| | | CGUUCGGCUCGCGUG | | | | | | | | |
| | | AGGC | | | | | | | | |
| hsa- | MI0005563 | UCUCCUCGAGGGGUC | 54 | MIMAT00 | ACCAGGAG | 7 | | | | chr14:100411 |
| miR- | | UCUGCCUCUACCCAG | | 04952 | GCUGAGGC | | | | | 123- |
| 665 | | GACUCUUUCAUGACC | | | CCCU | | | | | 100411194 |
| | | AGGAGGCUGAGGCCC | | | | | | | | |
| | | CUCACAGGCGGC | | | | | | | | |
| hsa- | MI0000098 | UGGCCGAUUUUGGCA | 55 | MIMAT00 | UUUGGCAC | 8 | MIMAT00 | AAUCAUGU | 9 | chr7:1292017 |
| miR-96 | | CUAGCACAUUUUUGC | | 00095 | UAGCACAU | | 04510 | GCAGUGCC | | 68-129201845 |
| | | UUGUGUCUCUCCGCU | | | UUUUGCU | | | AAUAUG | | |
| | | CUGAGCAAUCAUGUG | | | | | | | | |
| | | CAGUGCCAAUAUGGG | | | | | | | | |
| | | AAA | | | | | | | | |
| hsa- | MI0003672 | CCUUCCGGCGUCCCA | 56 | MIMAT00 | AGGCGGGG | 10 | | | | chr20:261368 |
| miR- | | GGCGGGGCGCCGCGG | | 03326 | CGCCGCGG | | | | | 22-26136914 |
| 663a | | GACCGCCCUCGUGUC | | | GACCGC | | | | | |
| | | UGUGGCGGUGGGAUC | | | | | | | | |
| | | CCGCGGCCGUGUUUU | | | | | | | | |
| | | CCUGGUGGCCCGGCC | | | | | | | | |
| | | AUG | | | | | | | | |
| hsa- | MI0000272 | GAGCUGCUUGCCUCC | 57 | MIMAT00 | UUUGGCAA | 11 | MIMAT00 | UGGUUCUA | 12 | chr7:1291974 |
| miR- | | CCCCGUUUUUGGCAA | | 00259 | UGGUAGAA | | 00260 | GACUUGCC | | 59-129197568 |
| 182 | | UGGUAGAACUCACAC | | | CUCACACU | | | AACUA | | |
| | | UGGUGAGGUAACAGG | | | | | | | | |
| | | AUCCGGUGGUUCUAG | | | | | | | | |
| | | ACUUGCCAACUAUGG | | | | | | | | |
| | | GGCGAGGACUCAGCC | | | | | | | | |
| | | GGCAC | | | | | | | | |
| hsa- | MI0003134 | GAUACUCGAAGGAGA | 58 | MIMAT00 | UGAAACAU | 13 | | | | chr14:100565 |
| miR- | | GGUUGUCCGUGUUGU | | 02816 | ACACGGGA | | | | | 724- |
| 494 | | CUUCUCUUUAUUUAU | | | AACCUC | | | | | 100565804 |
| | | GAUGAAACAUACACG | | | | | | | | |
| | | GGAAACCUCUUUUUU | | | | | | | | |
| | | AGUAUC | | | | | | | | |
| hsa- | MI0000253 | GAGGCAAAGUUCUGA | 59 | MIMAT00 | AAAGUUCU | 14 | MIMAT00 | UCAGUGCA | 15 | chr7:2595606 |
| miR- | | GACACUCCGACUCUG | | 04549 | GAGACACU | | 00243 | CUACAGAA | | 4-25956131 |
| 148a | | AGUAUGAUAGAAGUC | | | CCGACU | | | CUUUGU | | |
| | | AGUGCACUACAGAAC | | | | | | | | |
| | | UUUGUCUC | | | | | | | | |
| hsa- | MI0006353 | GGUAGAAUUCCAGUG | 60 | MIMAT00 | UGGCCCUG | 16 | | | | chr12:473344 |
| miR- | | GCCCUGACUGAAGAC | | 05881 | ACUGAAGA | | | | | 94-47334580 |
| 1291 | | CAGCAGUUGUACUGU | | | CCAGCAGU | | | | | |
| | | GGCUGUUGGUUUCAA | | | | | | | | |
| | | GCAGAGGCCUAAAGG | | | | | | | | |
| | | ACUGUCUUCCUG | | | | | | | | |
| hsa- | MI0003615 | UUCUCACCCCCGCCU | 61 | MIMAT00 | GACACGGG | 17 | | | | chr9:1398526 |
| miR- | | GACACGGGCGACAGC | | 03270 | CGACAGCU | | | | | 92-139852789 |
| 602 | | UGCGGCCCGCUGUGU | | | GCGGCCC | | | | | |
| | | UCACUCGGGCCGAGU | | | | | | | | |
| | | GCGUCUCCUGUCAGG | | | | | | | | |
| | | CAAGGGAGAGCAGAG | | | | | | | | |
| | | CCCCCCUG | | | | | | | | |
| hsa- | MI0000272 | GAGCUGCUUGCCUCC | 57 | MIMAT00 | UUUGGCAA | 11 | MIMAT00 | UGGUUCUA | 12 | chr7:1291974 |
| miR- | | CCCCGUUUUUGGCAA | | 00259 | UGGUAGAA | | 00260 | GACUUGCC | | 59-129197568 |
| 182* | | UGGUAGAACUCACAC | | | CUCACACU | | | AACUA | | |
| | | UGGUGAGGUAACAGG | | | | | | | | |
| | | AUCCGGUGGUUCUAG | | | | | | | | |
| | | ACUUGCCAACUAUGG | | | | | | | | |
| | | GGCGAGGACUCAGCC | | | | | | | | |
| | | GGCAC | | | | | | | | |
| hsa- | MI0003646 | GCGGGCGGCCCCGCG | 62 | MIMAT00 | GUGCAUUG | 18 | MIMAT00 | CAGUGCCU | 19 | chr17:176578 |
| miR- | | GUGCAUUGCUGUUGC | | 03301 | CUGUUGCA | | 04811 | CGGCAGUG | | 75-17657970 |
| 33b* | | AUUGCACGUGUGUGA | | | UUGC | | | CAGCCC | | |
| | | GGCGGGUGCAGUGCC | | | | | | | | |
| | | UCGGCAGUGCAGCCC | | | | | | | | |
| | | GGAGCCGGCCCCUGG | | | | | | | | |
| | | CACCAC | | | | | | | | |
| hsa- | MI0009983 | UAUGUUCAACGGCCA | 63 | MIMAT00 | ACCGUGCA | 20 | | | | chr4:1174403 |
| miR- | | UGGUAUCCUGACCGU | | 09448 | AAGGUAGC | | | | | 30-117440373 |
| 1973 | | GCAAAGGUAGCAUA | | | AUA | | | | | |
| hsa- | MI0000463 | CUCACAGCUGCCAGU | 64 | MIMAT00 | UUGCAUAG | 21 | | | | chr2:2198670 |
| miR- | | GUCAUUUUUGUGAUC | | 00439 | UCACAAAA | | | | | 77-219867166 |
| 153-1 | | UGCAGCUAGUAUUCU | | | GUGAUC | | | | | |
| | | CACUCCAGUUGCAUA | | | | | | | | |
| | | GUCACAAAAGUGAUC | | | | | | | | |
| | | AUUGGCAGGUGUGGC | | | | | | | | |
| hsa- | MI0000464 | AGCGGUGGCCAGUGU | 65 | MIMAT00 | UUGCAUAG | 21 | | | | chr7:1570597 |
| miR- | | CAUUUUUGUGAUGUU | | 00439 | UCACAAAA | | | | | 89-157059875 |
| 153-2 | | GCAGCUAGUAAUAUG | | | GUGAUC | | | | | |
| | | AGCCCAGUUGCAUAG | | | | | | | | |
| | | UCACAAAAGUGAUCA | | | | | | | | |
| | | UUGGAAACUGUG | | | | | | | | |
| hsa- | MI0000457 | CGGCCGGCCCUGGGU | 66 | MIMAT00 | CAUCUUCC | 22 | MIMAT00 | UAACACUG | 23 | chr12:694352 |
| miR- | | CCAUCUUCCAGUACA | | 04598 | AGUACAGU | | 00432 | UCUGGUAA | | 1-6943615 |
| 141* | | GUGUUGGAUGGUCUA | | | GUUGGA | | | AGAUGG | | |
| | | AUUGUGAAGCUCCUA | | | | | | | | |
| | | ACACUGUCUGGUAAA | | | | | | | | |
| | | GAUGGCUCCCGGGUG | | | | | | | | |
| | | GGUUC | | | | | | | | |
| hsa- | MI0007074 | CUCGGCGCGGGGCGC | 67 | MIMAT00 | CUCGGCGC | 24 | | | | chr15:946774 |
| miR- | | GGGCUCCGGGUUGGG | | 07347 | GGGGCGCG | | | | | 94-94677540 |
| 1469 | | GCGAGCCAACGCCGG | | | GGCUCC | | | | | |
| | | GG | | | | | | | | |
| hsa- | MI0000285 | AAAGAUCCUCAGACA | 68 | MIMAT00 | UCCUUCAU | 25 | MIMAT00 | GAUUUCAG | 26 | chr1:2076721 |
| miR- | | AUCCAUGUGCUUCUC | | 00266 | UCCACCGG | | 09197 | UGGAGUGA | | 01-207672210 |
| 205 | | UUGUCCUUCAUUCCA | | | AGUCUG | | | AGUUC | | |
| | | CCGGAGUCUGUCUCA | | | | | | | | |
| | | UACCCAACCAGAUUU | | | | | | | | |
| | | CAGUGGAGUGAAGUU | | | | | | | | |
| | | CAGGAGGCAUGGAGC | | | | | | | | |
| | | UGACA | | | | | | | | |
| hsa- | MI0006274 | UCCACUGCUGCCGCC | 69 | MIMAT00 | CCGUCGCC | 27 | | | | chr19:103751 |
| miR- | | GUCGCCGCCACCCGA | | 05826 | GCCACCCG | | | | | 34-10375214 |
| 1181 | | GCCGGAGCGGGCUGG | | | AGCCG | | | | | |
| | | GCCGCCAAGGCAAGA | | | | | | | | |
| | | UGGUGGACUACAGCG | | | | | | | | |
| | | UGUGGG | | | | | | | | |
| hsa- | MI0015997 | AAGGUUGCGGUGCAU | 70 | MIMAT00 | GCAUGUGA | 28 | MIMAT00 | ACUGUAAA | 29 | chr5:8595207 |
| miR- | | GUGAUGAAGCAAAUC | | 17984 | UGAAGCAA | | 17985 | CGCUUUCU | | 0-85952148 |
| 3607 | | AGUAUGAAUGAAUUC | | | AUCAGU | | | GAUG | | |
| | | AUGAUACUGUAAACG | | | | | | | | |
| | | CUUUCUGAUGUACUA | | | | | | | | |
| | | CUCA | | | | | | | | |
| hsa- | MI0001648 | CUGUGUGUGAUGAGC | 71 | MIMAT00 | UGGCAGUG | 30 | | | | chr5:5450211 |
| miR- | | UGGCAGUGUAUUGUU | | 01541 | UAUUGUUA | | | | | 7-54502207 |
| 449a | | AGCUGGUUGAAUAUG | | | GCUGGU | | | | | |
| | | UGAAUGGCAUCGGCU | | | | | | | | |
| | | AACAUGCAACUGCUG | | | | | | | | |
| | | UCUUAUUGCAUAUAC | | | | | | | | |
| | | A | | | | | | | | |
| hsa- | MI0000450 | ACAAUGCUUUGCUAG | 72 | MIMAT00 | UUUGGUCC | 31 | | | | chr18:176596 |
| miR- | | AGCUGGUAAAAUGGA | | 00427 | CCUUCAAC | | | | | 57-17659744 |
| 133a-1 | | ACCAAAUCGCCUCUU | | | CAGCUG | | | | | |
| | | CAAUGGAUUUGGUCC | | | | | | | | |
| | | CCUUCAACCAGCUGU | | | | | | | | |
| | | AGCUAUGCAUUGA | | | | | | | | |
| hsa- | MI0000451 | GGGAGCCAAAUGCUU | 73 | MIMAT00 | UUUGGUCC | 31 | | | | chr20:605725 |
| miR- | | UGCUAGAGCUGGUAA | | 00427 | CCUUCAAC | | | | | 64-60572665 |
| 133a-2 | | AAUGGAACCAAAUCG | | | CAGCUG | | | | | |
| | | ACUGUCCAAUGGAUU | | | | | | | | |
| | | UGGUCCCCUUCAACC | | | | | | | | |
| | | AGCUGUAGCUGUGCA | | | | | | | | |
| | | UUGAUGGCGCCG | | | | | | | | |
| hsa- | MI0000822 | CCUCAGAAGAAAGAU | 74 | MIMAT00 | UUUGGUCC | 32 | | | | chr6:5212168 |
| miR- | | GCCCCCUGCUCUGGC | | 00770 | CCUUCAAC | | | | | 0-52121798 |
| 133b | | UGGUCAAACGGAACC | | | CAGCUA | | | | | |
| | | AAGUCCGUCUUCCUG | | | | | | | | |
| | | AGAGGUUUGGUCCCC | | | | | | | | |
| | | UUCAACCAGCUACAG | | | | | | | | |
| | | CAGGGCUGGCAAUGC | | | | | | | | |
| | | CCAGUCCUUGGAGA | | | | | | | | |
| hsa- | MI0000286 | ACCCGGCAGUGCCUC | 75 | MIMAT00 | CUGUGCGU | 33 | | | | chr11:558089- |
| miR- | | CAGGCGCAGGGCAGC | | 00267 | GUGACAGC | | | | | 558198 |
| 210 | | CCCUGCCCACCGCAC | | | GGCUGA | | | | | |
| | | ACUGCGCUGCCCCAG | | | | | | | | |
| | | ACCCACUGUGCGUGU | | | | | | | | |
| | | GACAGCGGCUGAUCU | | | | | | | | |
| | | GUGCCUGGGCAGCGC | | | | | | | | |
| | | GACCC | | | | | | | | |
| hsa- | MI0000786 | AGGGCUCCUGACUCC | 76 | MIMAT00 | CUCCUGAC | 34 | MIMAT00 | ACUGGACU | 35 | chr5:1490925 |
| miR- | | AGGUCCUGUGUGUUA | | 00731 | UCCAGGUC | | 00732 | UGGAGUCA | | 81-149092646 |
| 378a | | CCUAGAAAUAGCACU | | | CUGUGU | | | GAAGG | | |
| | | GGACUUGGAGUCAGA | | | | | | | | |
| | | AGGCCU | | | | | | | | |
| hsa- | MI0000746 | GGCACCCACCCGUAG | 77 | MIMAT00 | CACCCGUA | 36 | MIMAT00 | CAAGCUCG | 37 | chr19:568876 |
| miR- | | AACCGACCUUGCGGG | | 00689 | GAACCGAC | | 04678 | UGUCUGUG | | 77-56887746 |
| 99b* | | GCCUUCGCCGCACAC | | | CUUGCG | | | GGUCCG | | |
| | | AAGCUCGUGUCUGUG | | | | | | | | |
| | | GGUCCGUGUC | | | | | | | | |
| hsa- | MI0000651 | UGGGAAACAUACUUC | 78 | MIMAT00 | UGGAAUGU | 38 | | | | chr20:605619 |
| miR-1-1 | | UUUAUAUGCCCAUAU | | 00416 | AAAGAAGU | | | | | 58-60562028 |
| | | GGACCUGCUAAGCUA | | | AUGUAU | | | | | |
| | | UGGAAUGUAAAGAAG | | | | | | | | |
| | | UAUGUAUCUCA | | | | | | | | |
| hsa- | MI0000437 | ACCUACUCAGAGUAC | 79 | MIMAT00 | UGGAAUGU | 38 | | | | chr18:176629 |
| miR-1-2 | | AUACUUCUUUAUGUA | | 00416 | AAAGAAGU | | | | | 63-17663047 |
| | | CCCAUAUGAACAUAC | | | AUGUAU | | | | | |
| | | AAUGCUAUGGAAUGU | | | | | | | | |
| | | AAAGAAGUAUGUAUU | | | | | | | | |
| | | UUUGGUAGGC | | | | | | | | |
| hsa- | MI0001641 | CGCCGGCCGAUGGGC | 80 | MIMAT00 | UAAUACUG | 39 | | | | chr1:1094248- |
| miR- | | GUCUUACCAGACAUG | | 01536 | UCUGGUAA | | | | | 1094330 |
| 429 | | GUUAGACCUGGCCCU | | | AACCGU | | | | | |
| | | CUGUCUAAUACUGUC | | | | | | | | |
| | | UGGUAAAACCGUCCA | | | | | | | | |
| | | UCCGCUGC | | | | | | | | |
| hsa- | MI0000261 | GUGUAUUCUACAGUG | 81 | MIMAT00 | UCUACAGU | 40 | MIMAT00 | GGAGACGC | 41 | chr11:720037 |
| miR- | | CACGUGUCUCCAGUG | | 00250 | GCACGUGU | | 04552 | GGCCCUGU | | 55-72003822 |
| 139 | | UGGCUCGGAGGCUGG | | | CUCCAG | | | UGGAGU | | |
| | | AGACGCGGCCCUGUU | | | | | | | | |
| | | GGAGUAAC | | | | | | | | |
| hsa- | MI0000484 | UGCUCCCUCUCUCAC | 82 | MIMAT00 | CAUCCCUU | 42 | MIMAT00 | CUCCCACA | 43 | chrX:4965484 |
| miR- | | AUCCCUUGCAUGGUG | | 00457 | GCAUGGUG | | 04613 | UGCAGGGU | | 9-49654934 |
| 188 | | GAGGGUGAGCUUUCU | | | GAGGG | | | UUGCA | | |
| | | GAAAACCCCUCCCAC | | | | | | | | |
| | | AUGCAGGGUUUGCAG | | | | | | | | |
| | | GAUGGCGAGCC | | | | | | | | |
| hsa- | MI0003560 | CGGGCCCCGGGCGGG | 83 | MIMAT00 | AGGGACGG | 44 | MIMAT00 | UAUUGCAC | 45 | chr1:1534315 |
| miR- | | CGGGAGGGACGGGAC | | 04792 | GACGCGGU | | 03218 | UCGUCCCG | | 92-153431687 |
| 92b | | GCGGUGCAGUGUUGU | | | GCAGUG | | | GCCUCC | | |
| | | UUUUUCCCCCGCCAA | | | | | | | | |
| | | UAUUGCACUCGUCCC | | | | | | | | |
| | | GGCCUCCGGCCCCCC | | | | | | | | |
| | | CGGCCC | | | | | | | | |
| hsa- | MI0003589 | AUCUGUGCUCUUUGA | 84 | MIMAT00 | UUACAGUU | 46 | MIMAT00 | UAACUGGU | 47 | chr5:5903518 |
| miR- | | UUACAGUUGUUCAAC | | 03247 | GUUCAACC | | 04797 | UGAACAAC | | 9-59035286 |
| 582 | | CAGUUACUAAUCUAA | | | AGUUACU | | | UGAACC | | |
| | | CUAAUUGUAACUGGU | | | | | | | | |
| | | UGAACAACUGAACCC | | | | | | | | |
| | | AAAGGGUGCAAAGUA | | | | | | | | |
| | | GAAACAUU | | | | | | | | |
| hsa- | MI0000342 | CCAGCUCGGGCAGCC | 85 | MIMAT00 | CAUCUUAC | 48 | MIMAT00 | UAAUACUG | 49 | chr1:1092347- |
| miR- | | GUGGCCAUCUUACUG | | 04571 | UGGGCAGC | | 00318 | CCUGGUAA | | 1092441 |
| 200b | | GGCAGCAUUGGAUGG | | | AUUGGA | | | UGAUGA | | |
| | | AGUCAGGUCUCUAAU | | | | | | | | |
| | | ACUGCCUGGUAAUGA | | | | | | | | |
| | | UGACGGCGGAGCCCU | | | | | | | | |
| | | GCACG | | | | | | | | |
| hsa- | MI0000273 | CCGCAGAGUGUGACU | 52 | MIMAT00 | UAUGGCAC | 4 | MIMAT00 | GUGAAUUA | 5 | chr7:1292019 |
| miR- | | CCUGUUCUGUGUAUG | | 00261 | UGGUAGAA | | 04560 | CCGAAGGG | | 81-129202090 |
| 183* | | GCACUGGUAGAAUUC | | | UUCACU | | | CCAUAA | | |
| | | ACUGUGAACAGUCUC | | | | | | | | |
| | | AGUCAGUGAAUUACC | | | | | | | | |
| | | GAAGGGCCAUAAACA | | | | | | | | |
| | | GAGCAGAGACAGAUC | | | | | | | | |
| | | CACGA | | | | | | | | |

Table 18 lists all the biomarkers useful with the invention (from Table 1 and Table 2). Table 18 provides the accession number and sequence (according to miRBase) for the precursor hairpin, as well as the mature, processed miRNAs (for both the 5′ and 3′ arm of the hairpin, where applicable). Additionally, the genomic location of the hairpin is also provided.

### Columns

(i) The “miRNA name” column is as described above.

(ii) The “Hairpin accession” column gives the unique number of the precursor hairpin, which is processed biologically, to yield the mature human miRNA, as provided by the specialist database, miRBase. The name is correct to miRBase version 16 (released, August 2010).

(iii) The “Hairpin sequence” column gives the sequence information of the precursor hairpin, which is processed biologically, to yield the mature human miRNA, as provided by the specialist database, miRBase. The name is correct to miRBase version 16 (released, August 2010).

(iv) The “Mature accession (−5p)” column gives the unique number of the mature, processed, miRNA located on the 5′ arm, as provided by the specialist database, miRBase. The name is correct to miRBase version 16 (released, August 2010).

(v) The “Mature sequence (−5p)” column gives the sequence information of the mature, processed, miRNA located on the 5′ arm, as provided by the specialist database, miRBase. The name is correct to miRBase version 16 (released, August 2010).

(vi) The “Mature accession (−3p)” column gives the unique number of the mature, processed, miRNA located on the 3′ arm, as provided by the specialist database, miRBase. The name is correct to miRBase version 16 (released, August 2010).

(vii) The “Mature sequence (−3p)” column gives the sequence information of the mature, processed, miRNA located on the 3′ arm, as provided by the specialist database, miRBase. The name is correct to miRBase version 16 (released, August 2010).

(viii) The “Chromosomal location” column gives the exact genomic location for the precursor hairpin, as provided by the specialist database, miRBase. The name is correct to miRBase version 16 (released, August 2010).

TABLE 19

| miRNA name⁽ⁱ⁾ | Assay ID⁽ⁱⁱ⁾ | No.⁽ⁱⁱⁱ⁾ |
|---|---|---|
| hsa-miR-1291 | 002838 | 16 |
| hsa-miR-449a | 001030 | 30 |
| hsa-miR-183 | 002269 | 4 |
| hsa-miR-1973 | 245468_mat | 20 |
| hsa-miR-3621 | 463091_mat | 1 |
| hsa-miR-665 | 002681 | 7 |
| hsa-miR-1-1 | 002222 | 38 |
| hsa-miR-133a-1 | 002246 | 31 |
| hsa-miR-133b | 002247 | 32 |

Table 19 lists the biomarkers used to assess the suitability of the claimed diagnostic and/or prognostic markers for detecting circulating miRNAs within human plasma and serum.

### Columns

(i) The “miRNA name” column gives the name of the human miRNA as provided by the specialist database, miRBase, according to version 16 (released, August 2010).

(ii) The “Assay ID” column gives the unique assay ID identifier used for ordering the specific TaqMan miRNA assay from Life Technologies. The assay ID is correct as of July 2013.

(iii) The SEQ ID NO: for the sequence of the mature, expressed miRNA biomarker, as shown in Table 18

TABLE 20

| | | | | | | | | Aggressive | Aggressive |
|---|---|---|---|---|---|---|---|---|---|
| | | | | | | | | vs indolent | vs indolent |
| | | | Correlates | Aggressive vs | Aggressive vs | Indolent vs | Indolent vs | & normal | and normal |
| | No. | Expression | with tissue | normal [BPH] | normal [BPH] | normal [BPH] | normal [BPH] | [BPH] log | [BPH] P- |
| miRNA name (i) | (ii) | (iii) | data (iv) | log fold change | P-value | log fold change | P-value | fold change | value |
| hsa-miR-665 | 7 | UP | Yes | 1.745 | 9.19E−06 | 2.324 | 2.03E−09 | 0.002 | 0.996 |
| hsa-miR-183 | 4 | UP | Yes | 1.026 | 0.016 | 1.107 | 0.004 | 0.196 | 0.553 |
| hsa-miR-3621 | 1 | UP | Yes | 1.482 | 0.111 | 1.542 | 0.067 | 0.326 | 0.638 |
| hsa-miR-1291 | 16 | UP | Yes | 0.476 | 0.574 | 1.418 | 0.067 | −0.587 | 0.359 |
| hsa-miR-133a-1 | 31 | DOWN | Yes | −2.426 | 0.003 | −0.076 | 0.913 | −2.369 | 1.02E−04 |
| hsa-miR-1-1 | 38 | DOWN | Yes | −1.950 | 0.017 | 0.071 | 0.920 | −2.004 | 0.001 |
| hsa-miR-449a | 30 | UP | No | −1.643 | 0.081 | 0.026 | 0.975 | −1.662 | 0.017 |
| hsa-miR-1973 | 20 | UP | No | −0.968 | 0.237 | −0.005 | 0.994 | −0.964 | 0.109 |
| hsa-miR-133b | 32 | DOWN | Yes | −1.650 | 0.047 | 0.847 | 0.252 | −2.284 | 3.77E−04 |

Table 20 lists the P-values and log fold changes of the miRNA markers used in the pilot prostate cancer plasma experiment, as described herein. The categories used in the analysis are: ‘Aggressive vs normal [BPH]’; ‘Indolent vs normal [BPH]’; ‘Aggressive vs indolent and normal [BPH]’ (i.e. aggressive samples vs every other sample). The differential expression profile of the biomarkers used in the plasma experiment is compared to their differential expression profile as reported in fresh PC tissue.

TABLE 21

| | | | | | | | | Aggressive | Aggressive |
|---|---|---|---|---|---|---|---|---|---|
| | | | | | | | | vs indolent | vs indolent |
| | | | Correlates | Aggressive vs | Aggressive vs | Indolent vs | Indolent vs | & normal | and normal |
| | No. | Expression | with tissue | normal [BPH] | normal [BPH] | normal [BPH] | normal [BPH] | [BPH] log | [BPH] P- |
| miRNA name (i) | (ii) | (iii) | data (iv) | log fold change | P-value | log fold change | P-value | fold change | value |
| hsa-miR-3621 | 1 | UP | Yes | 1.47 | 0.011 | 1.85 | 3.30E−08 | 0.545 | 0.426 |
| hsa-miR-665 | 7 | UP | Yes | 1.434 | 0.031 | 2.018 | 1.32E−07 | 0.425 | 0.581 |
| hsa-miR-1973 | 20 | UP | Yes | 0.978 | 0.192 | 1.29 | 0.002 | 0.333 | 0.666 |
| hsa-miR-1291 | 16 | UP | Yes | 0.237 | 0.808 | 1.326 | 0.012 | −0.427 | 0.662 |
| hsa-miR-183 | 4 | UP | Yes | 1.245 | 0.13 | 0.804 | 0.064 | 0.843 | 0.296 |
| hsa-miR-133a-1 | 31 | DOWN | No | 0.428 | 0.504 | 0.464 | 0.171 | 0.196 | 0.756 |
| hsa-miR-133b | 32 | DOWN | No | 0.953 | 0.385 | 0.589 | 0.309 | 0.658 | 0.532 |
| hsa-miR-1-1 | 38 | DOWN | No | 0.346 | 0.635 | 0.409 | 0.287 | 0.141 | 0.842 |
| hsa-miR-449a | 30 | UP | No | −1.286 | 0.385 | 0.896 | 0.252 | −1.733 | 0.224 |

Table 21 lists the P-values and log fold changes of the miRNA markers used in the pilot prostate cancer serum experiment, as described herein. The categories used in the analysis are: ‘Aggressive vs normal [BPH]’; ‘Indolent vs normal [BPH]’; ‘Aggressive vs indolent and normal [BPH]’ (i.e. aggressive samples vs every other sample). The differential expression profile of the biomarkers used in the serum experiment is compared to their differential expression profile as reported in fresh PC tissue.

### Columns (Tables 20 & 21)

(i) The “miRNA name” column gives the name of the human miRNA as provided by the specialist database, miRBase, according to version 16 (released, August 2010).

(ii) The SEQ ID NO: for the sequence of the mature, expressed miRNA biomarker, as shown in Table 18.

(iii) The direction of expression of the miRNA marker as previously reported for the prostate cancer fresh PC tissue data.

(iv) Correlation of the differential expression of the claimed miRNA markers in prostate cancer plasma/serum vs the differential expression of the same miRNA markers in prostate cancer fresh PC tissue.

TABLE 23

| miRNA name⁽ⁱ⁾ | No.⁽ⁱⁱ⁾ |
|---|---|
| hsa-miR-1-1/hsa-miR-1-2 | 38 |
| hsa-miR-96 | 8 |
| hsa-miR-141 | 22 |
| hsa-miR-153-1/hsa-miR-153-2 | 21 |
| hsa-miR-182 | 11 |
| hsa-miR-183 | 4 |
| hsa-miR-375 | 6 |
| hsa-miR-494 | 13 |
| hsa-miR-582 | 47 |
| hsa-miR-1291 | 16 |
| hsa-miR-1973 | 20 |
| hsa-miR-3621 | 1 |
| hsa-miR-133a-1/hsa-miR-133a-2 | 31 |
| hsa-miR-133b | 32 |
| hsa-miR-182* | 12 |
| hsa-miR-183* | 5 |
| hsa-miR-33b* | 19 |
| hsa-miR-99b* | 37 |

Table 23 lists the biomarkers used to assess the suitability of the claimed diagnostic and/or prognostic markers for PC in formalin-fixed paraffin-embedded (FFPE) samples.

### Columns

(i) The “miRNA name” column gives the name of the human miRNA as provided by the specialist database, miRBase, according to version 16 (released, August 2010).

(ii) The SEQ ID NO: for the sequence of the mature, expressed miRNA biomarker, as shown in Table 18.

TABLE 24
Preferred Subsets

hsa-miR-103, hsa-miR-1-1, hsa-miR-1181, hsa-miR-1291, hsa-miR-133a-1, hsa-miR-133b, hsa-miR-
141, hsa-miR-1469, hsa-miR-148*, hsa-miR-153, hsa-miR-182, hsa-miR-182*, hsa-miR-183, hsa-miR-
183*, hsa-miR-185, hsa-miR-191, hsa-miR-192, hsa-miR-1973, hsa-miR-200b, hsa-miR-205, hsa-miR-
210, hsa-miR-33b*, hsa-miR-3607-5p, hsa-miR-3621, hsa-miR-375, hsa-miR-378a, hsa-miR-429, hsa-
miR-494, hsa-miR-582, hsa-miR-602, hsa-miR-665, hsa-miR-96, hsa-miR-99b*.
hsa-miR-103, hsa-miR-1-1, hsa-miR-1181, hsa-miR-1291, hsa-miR-133a-1, hsa-miR-133b, hsa-miR-
141, hsa-miR-1469, hsa-miR-148*, hsa-miR-153, hsa-miR-182, hsa-miR-183*, hsa-miR-185, hsa-miR-
191, hsa-miR-192, hsa-miR-1973, hsa-miR-200b, hsa-miR-210, hsa-miR-33b*, hsa-miR-3607-5p, hsa-
miR-3621, hsa-miR-375, hsa-miR-378a, hsa-miR-429, hsa-miR-494, hsa-miR-582, hsa-miR-602, hsa-
miR-665, hsa-miR-96, hsa-miR-99b*
hsa-miR-103, hsa-miR-1-1, hsa-miR-1181, hsa-miR-1291, hsa-miR-133a-1, hsa-miR-133b, hsa-miR-
141, hsa-miR-1469, hsa-miR-148*, hsa-miR-153, hsa-miR-183*, hsa-miR-185, hsa-miR-191, hsa-miR-
192, hsa-miR-1973, hsa-miR-200b, hsa-miR-210, hsa-miR-33b*, hsa-miR-3607-5p, hsa-miR-3621, hsa-
miR-375, hsa-miR-378a, hsa-miR-429, hsa-miR-494, hsa-miR-582, hsa-miR-602, hsa-miR-665, hsa-miR-
96, hsa-miR-99b*
hsa-miR-1181, hsa-miR-1291, hsa-miR-1469, hsa-miR-153, hsa-miR-182, hsa-miR-182*, hsa-miR-183,
hsa-miR-183*, hsa-miR-1973, hsa-miR-205, hsa-miR-33b*, hsa-miR-3607-5p, hsa-miR-3621, hsa-miR-
375, hsa-miR-602 and hsa-miR-665, hsa-miR-96
hsa-miR-1181, hsa-miR-1291, hsa-miR-1469, hsa-miR-153, hsa-miR-183*, hsa-miR-1973, hsa-miR-
33b*, hsa-miR-3607-5p, hsa-miR-3621, hsa-miR-602, hsa-miR-665.
hsa-miR-103, hsa-miR-1-1, hsa-miR-1291, hsa-miR-133a-1, hsa-miR-133b, hsa-miR-141, hsa-miR-
148*, hsa-miR-182, hsa-miR-183, hsa-miR-183*, hsa-miR-185, hsa-miR-191, hsa-miR-192, hsa-miR-
1973, hsa-miR-200b, hsa-miR-210, hsa-miR-3607-5p, hsa-miR-3621, hsa-miR-375, hsa-miR-378a, hsa-
miR-429, hsa-miR-494, hsa-miR-582, hsa-miR-665, hsa-miR-96, hsa-miR-99b*.
hsa-miR-103, hsa-miR-1-1, hsa-miR-1291, hsa-miR-133a-1, hsa-miR-133b, hsa-miR-141, hsa-miR-
148*, hsa-miR-182, hsa-miR-183*, hsa-miR-185, hsa-miR-191, hsa-miR-192, hsa-miR-1973, hsa-miR-
200b, hsa-miR-210, hsa-miR-3607-5p, hsa-miR-3621, hsa-miR-375, hsa-miR-378a, hsa-miR-429, hsa-
miR-494, hsa-miR-582, hsa-miR-665, hsa-miR-96, hsa-miR-99b*.
hsa-miR-103, hsa-miR-1-1, hsa-miR-1291, hsa-miR-133a-1, hsa-miR-133b, hsa-miR-141, hsa-miR-
148*, hsa-miR-182, hsa-miR-183*, hsa-miR-185, hsa-miR-191, hsa-miR-192, hsa-miR-1973, hsa-miR-
200b, hsa-miR-210, hsa-miR-3607-5p, hsa-miR-3621, hsa-miR-375, hsa-miR-378a, hsa-miR-429, hsa-
miR-494, hsa-miR-582, hsa-miR-665, hsa-miR-96, hsa-miR-99b*.
hsa-miR-1291, hsa-miR-133a-1, hsa-miR-133b, hsa-miR-139, hsa-miR-182, hsa-miR-182*, hsa-miR-
183, hsa-miR-183*, hsa-miR-188-3p, hsa-miR-1973, hsa-miR-200b, hsa-miR-210, hsa-miR-3621, hsa-
miR-378a, hsa-miR-429, hsa-miR-449a, hsa-miR-582, hsa-miR-96, hsa-miR-99b*
hsa-miR-1291, hsa-miR-133a-1, hsa-miR-133b, hsa-miR-139139, hsa-miR-182, hsa-miR-182*, hsa-miR-
183, hsa-miR-183*, hsa-miR-188-3p, hsa-miR-1973, hsa-miR-200b, hsa-miR-3621, hsa-miR-378a, hsa-
miR-429, hsa-miR-582, hsa-miR-96, hsa-miR-99b*
hsa-miR-133a-1, hsa-miR-133b, hsa-miR-139139, hsa-miR-182, hsa-miR-182*, hsa-miR-183, hsa-miR-
183*, hsa-miR-188-3p, hsa-miR-200b, hsa-miR-210, hsa-miR-378a, hsa-miR-429, hsa-miR-449a, hsa-
miR-96, hsa-miR-99b*
hsa-miR-133a-1, hsa-miR-133b, hsa-miR-139139, hsa-miR-182, hsa-miR-182*, hsa-miR-183, hsa-miR-
183*, hsa-miR-188-3p, hsa-miR-200b, hsa-miR-378a, hsa-miR-429, miR-96, hsa-miR-99b*
hsa-miR-1291, hsa-miR-1973, hsa-miR-210, hsa-miR-3621, hsa-miR-449a, hsa-miR-582, hsa-miR-99b*
hsa-miR-3621, hsa-miR-665, hsa-miR-1291, hsa-miR-1973, hsa-miR-33b*, hsa-miR-3607-5p, hsa-miR-
1181, hsa-miR-1469, hsa-miR-602, hsa-miR-205, hsa-miR-183, hsa-miR-182*, hsa-miR-182,
hsa-miR-3621, hsa-miR-665, hsa-miR-1291, hsa-miR-1973, hsa-miR-33b*, hsa-miR-3607-5p, hsa-miR-
1181, hsa-miR-1469, hsa-miR-602
hsa-miR-3621, hsa-miR-153, hsa-miR-33b*, hsa-miR-1973, hsa-miR-183*, hsa-miR-96, hsa-miR-375,
hsa-miR-182, hsa-miR-183
hsa-miR-3621, hsa-miR-153, hsa-miR-33b*, hsa-miR-1973, hsa-miR-183*,
hsa-miR-665, hsa-miR-582, hsa-miR-182, hsa-miR-378a, hsa-miR-96, hsa-miR-200b, hsa-miR-191, hsa-
miR-429, hsa-miR-494, hsa-miR-99b*, hsa-miR-375, hsa-miR-141, hsa-miR-183*, hsa-miR-148*, hsa-
miR-1291, hsa-miR-185, hsa-miR-1973, hsa-miR-103, hsa-miR-133a-1, hsa-miR-3607-5p, hsa-miR-
133b, hsa-miR-1-1, hsa-miR-210
hsa-miR-3621, hsa-miR-1291, hsa-miR-1973, hsa-miR-449a
hsa-miR-99b*, hsa-miR-133b, hsa-miR-183*, hsa-miR-188-3p, hsa-miR-139139, hsa-miR-429, hsa-
miR-378a, hsa-miR-200b, hsa-miR-182*, hsa-miR-96, hsa-miR-133a-1, hsa-miR-183, hsa-miR-449a,
hsa-miR-210
hsa-miR-99b*, hsa-miR-133b, hsa-miR-183*, hsa-miR-188-3p, hsa-miR-139, hsa-miR-429, hsa-miR-
378a, hsa-miR-200b, hsa-miR-182*, hsa-miR-96, hsa-miR-133a-1, hsa-miR-183
hsa-miR-133b, hsa-miR-182, hsa-miR-183
hsa-miR-582, hsa-miR-99b*, hsa-miR-449a, hsa-miR-210
hsa-miR-3621, hsa-miR-1291, hsa-miR-1973, hsa-miR-449a
hsa-miR-3621, hsa-miR-665, hsa-miR-1291, hsa-miR-1973
hsa-miR-3621, hsa-miR-665
hsa-miR-3621, hsa-miR-33b*, hsa-miR-1973, hsa-miR-375, hsa-miR-182, hsa-miR-183, hsa-miR-602,
hsa-miR-1291, hsa-miR-103, hsa-miR-148*, hsa-miR-182*, hsa-miR-185, hsa-miR-191, hsa-miR-210,
hsa-miR-494, hsa-miR-582,
hsa-miR-3621, hsa-miR-183, hsa-miR-375, hsa-miR-665, hsa-miR-96, hsa-miR-663, hsa-miR-182, hsa-
miR-494, hsa-miR-148a*, hsa-miR-1291, hsa-miR-602, hsa-miR-182*, hsa-miR-33b*, hsa-miR-1973,
hsa-miR-153-1/hsa-miR-153-2, hsa-miR-141*, hsa-miR-1469, hsa-miR-1181 and hsa-miR-3607-5p
hsa-miR-3621, hsa-miR-665, hsa-miR-1291, hsa-miR-1973, hsa-miR-33b*, hsa-miR-3607-5p, hsa-miR-
1181, hsa-miR-1469 and hsa-miR-602.
hsa-miR-205 and hsa-miR-221
hsa-miR-153, hsa-miR-182, hsa-miR-183, hsa-miR-183*, hsa-miR-375 and hsa-miR-96
hsa-miR-153, hsa-miR-183*
hsa-miR-3621, hsa-miR-33b* and hsa-miR-1973
hsa-miR-183*, hsa-miR-185, hsa-miR-133a-1, hsa-miR-1-1
hsa-miR-665, hsa-miR-582, hsa-miR-182, hsa-miR-378a, hsa-miR-96, hsa-miR-200b, hsa-miR-191, hsa-
miR-429, hsa-miR-494, hsa-miR-99b*, hsa-miR-375, hsa-miR-141, hsa-miR-148*, hsa-miR-1291, hsa-
miR-1973, hsa-miR-103, hsa-miR-3607-5p, hsa-miR-133b and hsa-miR-210
hsa-miR-665, hsa-miR-3621, hsa-miR-1973, hsa-miR-1291, hsa-miR-192 and hsa-miR-183
hsa-miR-665, hsa-miR-3621, hsa-miR-1973, hsa-miR-1291 and hsa-miR-192.
hsa-miR-96, hsa-miR-182*, hsa-miR-449a, hsa-miR-210, hsa-miR-429, hsa-miR-188, hsa-miR-200b,
hsa-miR-183 and hsa-miR-183*
hsa-miR-183*, hsa-miR-188-3p, hsa-miR-429, hsa-miR-200b, hsa-miR-182*, hsa-miR-96 and hsa-miR-
183
hsa-miR-133a-1/hsa-miR-133a-2, hsa-miR-133b, hsa-miR-378aa, hsa-miR-99b*, hsa-miR-1-1/hsa-
miR-1-2, hsa-miR-139, hsa-miR-92b and hsa-miR-582
hsa-miR-99b*, hsa-miR-133b, hsa-miR-139, hsa-miR-378a and hsa-miR-133a-1.
hsa-miR-182 and hsa-miR-183
hsa-miR-133b
hsa-miR-582, hsa-miR-99b*, hsa-miR-449a and hsa-miR-210
hsa-miR-1291, hsa-miR-1973 and hsa-miR-449a
hsa-miR-3621
hsa-miR-1-1/hsa-miR-1-2, hsa-miR-96, hsa-miR-141, hsa-miR-153-1/hsa-miR-153-2, hsa-miR-182,
hsa-miR-183, hsa-miR-375, hsa-miR-494, hsa-miR-582, hsa-miR-1291, hsa-miR-1973, hsa-miR-3621,
hsa-miR-133a-1/hsa-miR-133a-2, hsa-miR-133b, hsa-miR-182*, hsa-miR-183*, hsa-miR-33b*, hsa-
miR-99b*

TABLE 25

| Diagnostic (PC vs BPH) | | | Prognostic (G8 vs G6) | | |
|---|---|---|---|---|---|
| | Log | | | Log | |
| miRNA name | FC | P-value | miRNA name | FC | P-value |
| hsa-miR-665 | −1.38 | 1.68E−04 | hsa-miR-582 | 1.83 | 4.41E−02 |
| hsa-miR-582 | −6.56 | 4.44E−10 | hsa-miR-99b* | 2.07 | 8.15E−02 |
| hsa-miR-182 | −2.57 | 4.44E−06 | hsa-miR-449a | 2.69 | 5.14E−03 |
| hsa-miR-378 | −1.13 | 6.35E−06 | hsa-miR-210 | 0.92 | 1.92E−02 |
| hsa-miR-96 | −4.02 | 2.16E−05 | | | |
| hsa-miR-200b | −1.96 | 6.17E−05 | | | |
| hsa-miR-191 | −1.67 | 1.00E−04 | | | |
| hsa-miR-429 | −2.84 | 1.87E−04 | | | |
| hsa-miR-494 | −1.86 | 2.33E−04 | | | |
| hsa-miR-99b* | −2.83 | 1.40E−03 | | | |
| hsa-miR-375 | −1.49 | 2.40E−03 | | | |
| hsa-miR-141 | −1.43 | 3.56E−03 | | | |
| hsa-miR-183* | 0.87 | 6.03E−03 | | | |
| hsa-miR-148* | −2.52 | 7.87E−03 | | | |
| hsa-miR-1291 | −2.01 | 7.95E−03 | | | |
| hsa-miR-185 | 0.84 | 1.07E−02 | | | |
| hsa-miR-1973 | −1.51 | 1.30E−02 | | | |
| hsa-miR-103 | −1.18 | 1.38E−02 | | | |
| hsa-miR-133a-1 | 1.23 | 1.47E−02 | | | |
| hsa-miR-3607-5p | −0.67 | 1.91E−02 | | | |
| hsa-miR-133b | −1.59 | 1.92E−02 | | | |
| hsa-miR-1-1 | 1.65 | 2.31E−02 | | | |
| hsa-miR-210 | −0.60 | 6.53E−02 | | | |

TABLE 26

| Diagnostic | | Prognostic (G8 vs | | | | | |
|---|---|---|---|---|---|---|---|
| (PC vs ctrl) | | G6) | | G6 vs Ctrl | | G8 vs Ctrl | |
| miRNA | | miRNA | | miRNA | | miRNA | Log |
| name | Log FC | name | Log FC | name | Log FC | name | FC |
| hsa-miR-665 | −2.05 | hsa-miR- | −0.65 | hsa-miR- | −1.63 | hsa-miR- | −2.27 |
| | | 3621 | | 3621 | | 3621 | |
| hsa-miR- | −1.74 | hsa-miR- | 0.84 | hsa-miR-665 | −2.16 | hsa-miR- | −2.04 |
| 3621 | | 1291 | | | | 665 | |
| hsa-miR- | −1.15 | hsa-miR- | 0.68 | hsa-miR- | −1.57 | | |
| 1973 | | 1973 | | 1291 | | | |
| hsa-miR- | −1.06 | hsa-miR- | 1.01 | hsa-miR- | −1.15 | | |
| 1291 | | 449a | | 1973 | | | |
| hsa-miR-192 | −0.67 | | | | | | |
| hsa-miR-183 | −0.84 | | | | | | |

TABLE 27

| | | First data set | | | | Second data set | | | |
|---|---|---|---|---|---|---|---|---|---|
| | | | sens- | spec- | | auc- | sens- | spec- | |
| size | names | auc-med | med | med | S + S | med | med | med | S + S |
| 2 | mir1-1 + mir582 | 92.61 | 91.67 | 90.91 | 1.83 | 87.57 | 76.00 | 92.86 | 1.69 |
| 2 | mir183* + mir582 | 94.89 | 95.83 | 90.91 | 1.87 | 87.00 | 72.00 | 92.86 | 1.65 |
| 2 | mir185 + mir582 | 92.42 | 100.00 | 86.36 | 1.86 | 88.14 | 88.00 | 82.14 | 1.70 |
| 2 | mir210 + mir582 | 88.45 | 95.83 | 86.36 | 1.82 | 87.14 | 80.00 | 85.71 | 1.66 |
| 3 | mir1- | 94.51 | 100.00 | 86.36 | 1.86 | 89.29 | 80.00 | 89.29 | 1.69 |
| | 1 + mir183* + mir582 | | | | | | | | |
| 3 | mir1- | 92.80 | 100.00 | 86.36 | 1.86 | 87.71 | 88.00 | 82.14 | 1.70 |
| | 1 + mir185 + mir582 | | | | | | | | |
| 3 | mir1- | 92.80 | 91.67 | 90.91 | 1.83 | 87.29 | 80.00 | 85.71 | 1.66 |
| | 1 + mir1973 + mir582 | | | | | | | | |
| 3 | mir1- | 92.61 | 91.67 | 90.91 | 1.83 | 87.71 | 76.00 | 89.29 | 1.65 |
| | 1 + mir221 + mir582 | | | | | | | | |
| 3 | mir1- | 92.23 | 100.00 | 86.36 | 1.86 | 86.57 | 88.00 | 82.14 | 1.70 |
| | 1 + mir33b* + mir582 | | | | | | | | |
| 3 | mir1- | 97.16 | 100.00 | 90.91 | 1.91 | 85.43 | 100.00 | 53.57 | 1.54 |
| | 1 + mir582 + mir96 | | | | | | | | |
| 3 | mir133a- | 95.27 | 95.83 | 90.91 | 1.87 | 85.43 | 72.00 | 89.29 | 1.61 |
| | 1 + mir183* + mir582 | | | | | | | | |
| 3 | mir133b + mir183* + | 94.89 | 95.83 | 90.91 | 1.87 | 86.43 | 72.00 | 89.29 | 1.61 |
| | mir582 | | | | | | | | |
| 3 | mir183* + mir185 + | 94.13 | 100.00 | 86.36 | 1.86 | 89.29 | 72.00 | 96.43 | 1.68 |
| | mir582 | | | | | | | | |
| 3 | mir183* + mir221 + | 94.32 | 100.00 | 86.36 | 1.86 | 87.57 | 72.00 | 96.43 | 1.68 |
| | mir582 | | | | | | | | |
| 3 | mir183* + mir33b* + | 94.32 | 95.83 | 90.91 | 1.87 | 87.57 | 92.00 | 75.00 | 1.67 |
| | mir582 | | | | | | | | |
| 3 | mir183* + mir375 + | 95.83 | 95.83 | 90.91 | 1.87 | 85.43 | 84.00 | 82.14 | 1.66 |
| | mir582 | | | | | | | | |
| 3 | mir183* + mir582 + | 94.70 | 95.83 | 90.91 | 1.87 | 87.29 | 72.00 | 96.43 | 1.68 |
| | mir665 | | | | | | | | |
| 3 | mir185 + mir210 + | 97.73 | 91.67 | 95.45 | 1.87 | 86.29 | 92.00 | 71.43 | 1.63 |
| | mir582 | | | | | | | | |
| 3 | mir185 + mir221 + | 94.51 | 100.00 | 86.36 | 1.86 | 86.86 | 92.00 | 75.00 | 1.67 |
| | mir582 | | | | | | | | |
| 3 | mir185 + mir33b* + | 92.61 | 100.00 | 86.36 | 1.86 | 88.43 | 88.00 | 82.14 | 1.70 |
| | mir582 | | | | | | | | |
| 3 | mir185 + mir375 + | 95.45 | 100.00 | 86.36 | 1.86 | 85.57 | 84.00 | 78.57 | 1.63 |
| | mir582 | | | | | | | | |
| 3 | mir185 + mir378 + | 97.54 | 100.00 | 90.91 | 1.91 | 86.86 | 92.00 | 71.43 | 1.63 |
| | mir582 | | | | | | | | |
| 3 | mir185 + mir582 + | 93.56 | 100.00 | 86.36 | 1.86 | 89.29 | 88.00 | 78.57 | 1.67 |
| | mir665 | | | | | | | | |
| 3 | mir185 + mir582 + | 96.59 | 100.00 | 90.91 | 1.91 | 87.29 | 72.00 | 92.86 | 1.65 |
| | mir96 | | | | | | | | |
| 3 | mir210 + mir582 + | 95.27 | 100.00 | 90.91 | 1.91 | 85.57 | 72.00 | 85.71 | 1.58 |
| | mir96 | | | | | | | | |

1. A method for analysing a subject sample, comprising a step of determining the level of at least one biomarker selected from: hsa-miR-3621 (SEQ ID NO:1) and the other 34 biomarkers in Table 17 in the sample, wherein the level of the biomarker provides a diagnostic indicator of whether the subject has prostate cancer and/or a prognostic indicator of whether the subject has prostate cancer in the aggressive form or indolent form.

2. The method of claim 1, wherein the levels of at least two Table 17 biomarkers (a ‘panel’) are measured in the sample.

3. The method of claim 2, wherein the panel comprises marker(s) from Table 1.

4. The method of claim 2, wherein the panel comprises marker(s) from Table 2.

5. The method of claim 2, wherein the panel includes (i) any one of the 34 biomarkers in Table 17 in combination with (ii) any of the other 33 biomarkers in Table 17.

6. The method of claim 2, wherein the panel is a panel from Tables 3 to 9 herein.

7. The method of claim 2, wherein the panel is a panel from Tables 10 to 16 herein.

8. The method of claim 1, wherein up to 7 biomarkers from Table 17 are measured.

9. The method of claim 1, including measurement of at least one of: (a) a known biomarker for PC, which may or may not be miRNA; and/or (b) other information about the subject; and/or (c) other diagnostic tests or clinical indicators for PC.

10. A method for diagnosing a subject as having PC, comprising steps of: (i) determining the levels of at least 2 biomarkers of Table 17 in a sample from the subject; and (ii) comparing the determination from step (i) to data obtained from samples from subjects without PC and/or from subjects with PC, wherein the comparison provides a diagnostic indicator of whether the subject has PC.

11. A method for monitoring development of PC in a subject, comprising steps of: (i) determining the levels of at least 1 biomarker of Table 17 in a first sample from the subject taken at a first time; and (ii) determining the levels of that biomarker of Table 17 in a second sample from the subject taken at a second time, wherein: (a) the second time is later than the first time; and (b) a change in the level(s) of the biomarker(s) in the second sample compared with the first sample indicates that PC is in remission or is progressing.

12. A device for the diagnosis and/or prognosis of PC, wherein the device permits determination of the level(s) of at least 1 Table 17 biomarker.

13. A kit comprising reagents for measuring the levels of at least 2 different Table 17 biomarkers.

14. The use of a Table 1 biomarker as a diagnostic biomarker for prostate cancer.

15. The use of a Table 2 biomarker as a prognostic biomarker for prostate cancer.


SEQUENCE LISTING (85 sequences)

SEQ ID NO: 1 - RNA, 20 nt, H. sapiens
cgcgggucggggucugcagg

SEQ ID NO: 2 - RNA, 22 nt, H. sapiens
accuggcauacaauguagauuu

SEQ ID NO: 3 - RNA, 23 nt, H. sapiens
agcuacauugucugcuggguuuc

SEQ ID NO: 4 - RNA, 22 nt, H. sapiens
uauggcacugguagaauucacu

SEQ ID NO: 5 - RNA, 22 nt, H. sapiens
gugaauuaccgaagggccauaa

SEQ ID NO: 6 - RNA, 22 nt, H. sapiens
uuuguucguucggcucgcguga

SEQ ID NO: 7 - RNA, 20 nt, H. sapiens
accaggaggcugaggccccu

SEQ ID NO: 8 - RNA, 23 nt, H. sapiens
uuuggcacuagcacauuuuugcu

SEQ ID NO: 9 - RNA, 22 nt, H. sapiens
aaucaugugcagugccaauaug

SEQ ID NO: 10 - DNA, 22 bp, H. sapiens
aggcggggcgccgcgggaccgc

SEQ ID NO: 11 - RNA, 24 nt, H. sapiens
uuuggcaaugguagaacucacacu

SEQ ID NO: 12 - RNA, 21 nt, H. sapiens
ugguucuagacuugccaacua

SEQ ID NO: 13 - RNA, 22 nt, H. sapiens
ugaaacauacacgggaaaccuc

SEQ ID NO: 14 - RNA, 22 nt, H. sapiens
aaaguucugagacacuccgacu

SEQ ID NO: 15 - RNA, 22 nt, H. sapiens
ucagugcacuacagaacuuugu

SEQ ID NO: 16 - RNA, 24 nt, H. sapiens
uggcccugacugaagaccagcagu

SEQ ID NO: 17 - RNA, 23 nt, H. sapiens
gacacgggcgacagcugcggccc

SEQ ID NO: 18 - RNA, 20 nt, H. sapiens
gugcauugcuguugcauugc

SEQ ID NO: 19 - RNA, 22 nt, H. sapiens
cagugccucggcagugcagccc

SEQ ID NO: 20 - RNA, 19 nt, H. sapiens
accgugcaaagguagcaua

SEQ ID NO: 21 - RNA, 22 nt, H. sapiens
uugcauagucacaaaagugauc

SEQ ID NO: 22 - RNA, 22 nt, H. sapiens
caucuuccaguacaguguugga

SEQ ID NO: 23 - RNA, 22 nt, H. sapiens
uaacacugucugguaaagaugg

SEQ ID NO: 24 - RNA, 22 nt, H. sapiens
cucggcgcggggcgcgggcucc

SEQ ID NO: 25 - RNA, 22 nt, H. sapiens
uccuucauuccaccggagucug

SEQ ID NO: 26 - RNA, 21 nt, H. sapiens
gauuucaguggagugaaguuc

SEQ ID NO: 27 - RNA, 21 nt, H. sapiens
ccgucgccgccacccgagccg

SEQ ID NO: 28 - RNA, 22 nt, H. sapiens
gcaugugaugaagcaaaucagu

SEQ ID NO: 29 - RNA, 20 nt, H. sapiens
acuguaaacgcuuucugaug

SEQ ID NO: 30 - RNA, 22 nt, H. sapiens
uggcaguguauuguuagcuggu

SEQ ID NO: 31 - RNA, 22 nt, H. sapiens
uuugguccccuucaaccagcug

SEQ ID NO: 32 - RNA, 22 nt, H. sapiens
uuugguccccuucaaccagcua

SEQ ID NO: 33 - RNA, 22 nt, H. sapiens
cugugcgugugacagcggcuga

SEQ ID NO: 34 - RNA, 22 nt, H. sapiens
cuccugacuccagguccugugu

SEQ ID NO: 35 - RNA, 21 nt, H. sapiens
acuggacuuggagucagaagg

SEQ ID NO: 36 - RNA, 22 nt, H. sapiens
cacccguagaaccgaccuugcg

SEQ ID NO: 37 - RNA, 22 nt, H. sapiens
caagcucgugucuguggguccg

SEQ ID NO: 38 - RNA, 22 nt, H. sapiens
uggaauguaaagaaguauguau

SEQ ID NO: 39 - RNA, 22 nt, H. sapiens
uaauacugucugguaaaaccgu

SEQ ID NO: 40 - RNA, 22 nt, H. sapiens
ucuacagugcacgugucuccag

SEQ ID NO: 41 - RNA, 22 nt, H. sapiens
ggagacgcggcccuguuggagu

SEQ ID NO: 42 - RNA, 21 nt, H. sapiens
caucccuugcaugguggaggg

SEQ ID NO: 43 - RNA, 21 nt, H. sapiens
cucccacaugcaggguuugca

SEQ ID NO: 44 - RNA, 22 nt, H. sapiens
agggacgggacgcggugcagug

SEQ ID NO: 45 - RNA, 22 nt, H. sapiens
uauugcacucgucccggccucc

SEQ ID NO: 46 - RNA, 23 nt, H. sapiens
uuacaguuguucaaccaguuacu

SEQ ID NO: 47 - RNA, 22 nt, H. sapiens
uaacugguugaacaacugaacc

SEQ ID NO: 48 - RNA, 22 nt, H. sapiens
caucuuacugggcagcauugga

SEQ ID NO: 49 - RNA, 22 nt, H. sapiens
uaauacugccugguaaugauga

SEQ ID NO: 50 - RNA, 85 nt, H. sapiens
gugagcugcuggggacgcgggucggggucugcagggcggugcggcagccgccaccugacgccgcgccuuugucugugucccacag

SEQ ID NO: 51 - RNA, 110 nt, H. sapiens
ugaacauccaggucuggggcaugaaccuggcauacaauguagauuucuguguucguuaggcaacagcuacauugucugcuggguuucaggcuaccuggaaacauguucuc

SEQ ID NO: 52 - RNA, 110 nt, H. sapiens
ccgcagagugugacuccuguucuguguauggcacugguagaauucacugugaacagucucagucagugaauuaccgaagggccauaaacagagcagagacagauccacga

SEQ ID NO: 53 - RNA, 64 nt, H. sapiens
ccccgcgacgagccccucgcacaaaccggaccugagcguuuuguucguucggcucgcgugaggc

SEQ ID NO: 54 - RNA, 72 nt, H. sapiens
ucuccucgaggggucucugccucuacccaggacucuuucaugaccaggaggcugaggccccucacaggcggc

SEQ ID NO: 55 - RNA, 78 nt, H. sapiens
uggccgauuuuggcacuagcacauuuuugcuugugucucuccgcucugagcaaucaugugcagugccaauaugggaaa

SEQ ID NO: 56 - RNA, 93 nt, H. sapiens
ccuuccggcgucccaggcggggcgccgcgggaccgcccucgugucuguggcggugggaucccgcggccguguuuuccugguggcccggccaug

SEQ ID NO: 57 - RNA, 110 nt, H. sapiens
gagcugcuugccuccccccguuuuuggcaaugguagaacucacacuggugagguaacaggauccggugguucuagacuugccaacuauggggcgaggacucagccggcac

SEQ ID NO: 58 - RNA, 81 nt, H. sapiens
gauacucgaaggagagguuguccguguugucuucucuuuauuuaugaugaaacauacacgggaaaccucuuuuuuaguauc

SEQ ID NO: 59 - RNA, 68 nt, H. sapiens
gaggcaaaguucugagacacuccgacucugaguaugauagaagucagugcacuacagaacuuugucuc

SEQ ID NO: 60 - RNA, 87 nt, H. sapiens
gguagaauuccaguggcccugacugaagaccagcaguuguacuguggcuguugguuucaagcagaggccuaaaggacugucuuccug

SEQ ID NO: 61 - RNA, 98 nt, H. sapiens
uucucacccccgccugacacgggcgacagcugcggcccgcuguguucacucgggccgagugcgucuccugucaggcaagggagagcagagccccccug

SEQ ID NO: 62 - RNA, 96 nt, H. sapiens
gcgggcggccccgcggugcauugcuguugcauugcacgugugugaggcgggugcagugccucggcagugcagcccggagccggccccuggcaccac

SEQ ID NO: 63 - RNA, 44 nt, H. sapiens
uauguucaacggccaugguauccugaccgugcaaagguagcaua

SEQ ID NO: 64 - RNA, 90 nt, H. sapiens
cucacagcugccagugucauuuuugugaucugcagcuaguauucucacuccaguugcauagucacaaaagugaucauuggcagguguggc

SEQ ID NO: 65 - RNA, 87 nt, H. sapiens
agcgguggccagugucauuuuugugauguugcagcuaguaauaugagcccaguugcauagucacaaaagugaucauuggaaacugug

SEQ ID NO: 66 - RNA, 95 nt, H. sapiens
cggccggcccuggguccaucuuccaguacaguguuggauggucuaauugugaagcuccuaacacugucugguaaagauggcucccggguggguuc

SEQ ID NO: 67 - RNA, 47 nt, H. sapiens
cucggcgcggggcgcgggcuccggguuggggcgagccaacgccgggg

SEQ ID NO: 68 - RNA, 110 nt, H. sapiens
aaagauccucagacaauccaugugcuucucuuguccuucauuccaccggagucugucucauacccaaccagauuucaguggagugaaguucaggaggcauggagcugaca

SEQ ID NO: 69 - RNA, 81 nt, H. sapiens
uccacugcugccgccgucgccgccacccgagccggagcgggcugggccgccaaggcaagaugguggacuacagcguguggg

SEQ ID NO: 70 - RNA, 79 nt, H. sapiens
aagguugcggugcaugugaugaagcaaaucaguaugaaugaauucaugauacuguaaacgcuuucugauguacuacuca

SEQ ID NO: 71 - RNA, 91 nt, H. sapiens
cugugugugaugagcuggcaguguauuguuagcugguugaauaugugaauggcaucggcuaacaugcaacugcugucuuauugcauauaca

SEQ ID NO: 72 - RNA, 88 nt, H. sapiens
acaaugcuuugcuagagcugguaaaauggaaccaaaucgccucuucaauggauuugguccccuucaaccagcuguagcuaugcauuga

SEQ ID NO: 73 - RNA, 102 nt, H. sapiens
gggagccaaaugcuuugcuagagcugguaaaauggaaccaaaucgacuguccaauggauuugguccccuucaaccagcuguagcugugcauugauggcgccg

SEQ ID NO: 74 - RNA, 119 nt, H. sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 75 - RNA, 110 nt, H. sapiens
acccggcagugccuccaggcgcagggcagccccugcccaccgcacacugcgcugccccagacccacugugcgugugacagcggcugaucugugccugggcagcgcgaccc

SEQ ID NO: 76 - RNA, 66 nt, H. sapiens
agggcuccugacuccagguccuguguguuaccuagaaauagcacuggacuuggagucagaaggccu

SEQ ID NO: 77 - RNA, 70 nt, H. sapiens
ggcacccacccguagaaccgaccuugcggggccuucgccgcacacaagcucgugucuguggguccguguc

SEQ ID NO: 78 - RNA, 71 nt, H. sapiens
ugggaaacauacuucuuuauaugcccauauggaccugcuaagcuauggaauguaaagaaguauguaucuca

SEQ ID NO: 79 - RNA, 85 nt, H. sapiens
accuacucagaguacauacuucuuuauguacccauaugaacauacaaugcuauggaauguaaagaaguauguauuuuugguaggc

SEQ ID NO: 80 - RNA, 83 nt, H. sapiens
cgccggccgaugggcgucuuaccagacaugguuagaccuggcccucugucuaauacugucugguaaaaccguccauccgcugc

SEQ ID NO: 81 - RNA, 68 nt, H. sapiens
guguauucuacagugcacgugucuccaguguggcucggaggcuggagacgcggcccuguuggaguaac

SEQ ID NO: 82 - RNA, 86 nt, H. sapiens
ugcucccucucucacaucccuugcaugguggagggugagcuuucugaaaaccccucccacaugcaggguuugcaggauggcgagcc

SEQ ID NO: 83 - RNA, 96 nt, H. sapiens
cgggccccgggcgggcgggagggacgggacgcggugcaguguuguuuuuucccccgccaauauugcacucgucccggccuccggcccccccggccc

SEQ ID NO: 84 - RNA, 98 nt, H. sapiens
aucugugcucuuugauuacaguuguucaaccaguuacuaaucuaacuaauuguaacugguugaacaacugaacccaaagggugcaaaguagaaacauu

SEQ ID NO: 85 - RNA, 95 nt, H. sapiens
ccagcucgggcagccguggccaucuuacugggcagcauuggauggagucaggucucuaauacugccugguaaugaugacggcggagcccugcacg